(19) United States
(12) Patent Application Publication
(10) Pub. No.: US 2024/0352154 A1
(43) Pub. Date: Oct. 24, 2024

(54) METHODS AND COMPOSITIONS RELATING TO HIGH-THROUGHPUT MODELS FOR ANTIBODY DISCOVERY AND/OR OPTIMIZATION

(71) Applicant: THE CHILDREN'S MEDICAL CENTER CORPORATION, Boston, MA (US)

(72) Inventors: Frederick W. ALT, Cambridge, MA (US); Suvi JAIN, Boston, MA (US); Zhaoqing BA, Boston, MA (US); Ming TIAN, Boston, MA (US)

(73) Assignee: THE CHILDREN'S MEDICAL CENTER CORPORATION, Boston, MA (US)

(21) Appl. No.: 18/607,961

(22) Filed: Mar. 18, 2024

Related U.S. Application Data
(62) Division of application No. 16/973,125, filed on Dec. 8, 2020, now abandoned.
(60) Provisional application No. 62/684,367, filed on Jun. 13, 2018.

(51) Int. Cl. C07K 16/46; A01K 67/0278; C12N 5/0735; C12N 15/85

(52) U.S. Cl. CPC C07K 16/461; A01K 67/0278; C12N 5/0606; C12N 15/8509; A01K 2207/15; A01K 2217/072; A01K 2227/105; C07K 2317/10

(57) ABSTRACT

Described herein are compositions (e.g. cells and transgenic animals) and methods relating to engineered Ig loci that permit expression of particular antibodies or antibody segments while still permitting recombination and/or maturation process for antibody optimization.

### CROSS-REFERENCE TO RELATED APPLICATIONS

This application is a divisional under 35 U.S.C. of § 121 of co-pending U.S. Ser. No. 16/973,125 filed Dec. 8, 2020, which is a 35 U.S.C. § 371 National Phase Entry Application of International Application No. PCT/US2019/036321 filed Jun. 10, 2019, which designates the U.S. and claims benefit under 35 U.S.C. § 119(e) of U.S. Provisional Application No. 62/684,367 filed Jun. 13, 2018, the contents of which are incorporated herein by reference in their entireties.

### GOVERNMENT SUPPORT

This invention was made with government support under Grant Number AI020047 and AI100645 awarded by the National Institutes of Health. The Government has certain rights in the invention.

### SEQUENCE LISTING

The instant application contains a Sequence Listing which has been submitted electronically in XML file format and is hereby incorporated by reference in its entirety. Said XML copy, created on Mar. 15, 2024, is named 701039-092250USD1_SL.xml and is 91,877 bytes in size.

### FIELD OF THE INVENTION

The invention relates to engineered antibodies and methods of discovery and/or optimizing antibodies.

### BACKGROUND

The mammalian adaptive immune response relies upon antibodies. A healthy animal will produce a very large number of different antibodies, each of which can selectively bind to a different molecule, which is called an antigen. The binding of the antibody to an antigen triggers an immune response which allows the body to destroy the antigen. If the antigen is a molecule on a pathogen, this permits the body to counter the infection by attacking the pathogen.

Antibodies are comprised of two identical Ig heavy chain (IgH) polypeptides and two identical light chain (IgL) polypeptides. Portions of the IgH and IgL chains called the variable region form the antigen-binding site. The sequence of the antigen-binding site determines what antigen(s) the antibody can bind to and how tight that binding is. In order to have a robust immune response, it is important for an animal to have both a wide-variety of antigen-binding sites represented in the antibody population so that the body can recognize any given antigen, and a mechanism for affinity maturation of primary antibodies to improve the ability to recognize any given antigen.

The IgH variable regions are assembled in the genome of B cells from gene segments referred to as VH, D, and J_{H}. Counting only the functional gene segments, there are 39 VH, 25 D and 6 J_{H} segments in the human IgH locus. Prior to an antibody being expressed, the IgH gene will be subjected to a process called V(D)J recombination, in which 1 VH, 1 D, and 1 J_{H} segment are combined in a highly diverse fashion in order to create a nucleic acid sequence that encodes a mature antibody. The different combinations of V_{H}, D, and J_{H}, and in particular the way the edges of the V_{H}, D, and J_{H} segments are joined to each other contribute to the extensive diversity of antibodies present in an individual. The light chain present in the B cell will be undergoing a similar set of processes, and further diversity is generated by the pairing of unique light and heavy chains along with the diversity in the junction by which they are put together. Specifically, the Ig light chain present in the B cell is generated by a similar V(D)J recombination process at either the Igκ or Igλ light chain locus. In these loci joining of V_{L} and J_{L} segments similarly results in generation of diverse IgL chains by joining different VL and JL segments and by generating diversity in the junctions in which they are put together Further antibody diversity in the context of the pairing of unique Ig light and Ig heavy chains further diversifies antibody repertories. In general, most B cells express a single IgH and IgL pair out of the huge number that can be assembled in the total population of developing B cells. In this regard, the size of the potentially expressed antibody repertoire in an organism is limited by the total number of B cells it can generate at steady state.

If an antibody encounters a foreign antigen to which it can bind, the B cell which makes that particular antibody will be activated. This will cause the B cell to replicate and those resulting B cells can be subject to additional genomic alterations that can lead to further diversification/affinity maturation (e.g. via somatic hypermutation (SHM) or germinal center reaction (GC)) of their antibodies. The efficacy of an antibody depends upon its specificity and affinity toward a relevant antigen. As described above, both V(D)J recombination and SHM make important contributions in this respect but at different points in the evolution of the antibody. V(D)J recombination creates an enormous pool of antigen-binding sites, individually expressed on particular B cells in a steady-state B cell population, so that any potential antigen might find a reasonable match; once a matched B cell has been found, somatic hypermutation and the GC response fine-tune the antigen-binding site to perfect the antibody-antigen interaction.

By studying natural immune responses or peripheral B cell repertoires, it is possible to identify which V, D, and/or J segments are most frequently expressed and by extension have a strong chance of being involved in generating an immune response to particular antigens. However, the ability of current methods of antibody production to apply the power of V(D)J recombination, SHM, and GC processes (Lonberg, Nature Biotechnology 23, 1117 (2005)) to optimization of existing antibodies in mice is limited, e.g., by the very small overall repertoire of potentially expressed antibodies in mice versus humans due to the much smaller size of the mouse immune system, and correspondingly much smaller number of B cells and potential precursors for targeting a particular immune response.

### SUMMARY

The invention relates to, in significant part, a novel method to generate novel antibodies (e.g., therapeutic and/or human antibodies), using a novel engineered immune system, e.g., in mice, as well as a novel system/method to optimize existing therapeutic antibodies or newly discovered candidate antibodies. In some embodiments, the system and/or method relates to an engineered mouse immune system. The engineered immune system is modified to allow easy insertion of one or more non-native components into the Ig locus of a model cell of a model animal. The engineered immune system is modified to drive production of V(D)J recombinations with any desired component, such as a desired V_{H} and/or V_{L} segment. These segments can be taken, for example, from a known antibody (e.g., human antibody) that is in need of improvement, such as improved affinity, specificity, or breadth. In some embodiments of any of the aspects, the segments are frequently used segments in human antibody repertoire. In some embodiments of any of the aspects, the segments are human V segments with mouse D and J segments. In some embodiments of any of the aspects, mouse D and J segments are appropriate for most humanized antibodies for two reasons: 1. Ds are diverse and in the full antibody the V(D)J junctional region is usually extremely diversified by V(D)J joining mechanisms, sometimes leaving the Ds nearly unrecognizable in the final antibody; 2. J_{H} segments are highly homologous in mouse and human; 3. SHM can mature the entire V(D)J segment including the antigen contact CDR1, CDR2 and CDR3 V(D)J junction in mature B cells during germinal center (GC) reaction. Some embodiments involve expressing precursor IgH and IgH V exons specifically in peripheral or GC B cells to allow them to escape potential tolerance control (e.g., central tolerance control), so that they can be optimized specifically by SHM in peripheral germinal center (GC) B cell responses. The system can be carried out in a model animal, such as a mouse. Moreover, the engineered immune system can be used for optimizing antigens, e.g. for testing sequential immunization strategies for optimization of bnAbs.

The invention is based, at least in part, on the discovery that which IgH locus V segment is most strongly subject to V(D)J recombination can be controlled by providing non-native CBE sequences in an engineered Ig locus, for example by providing a CBE to the most proximal IgH VH5-1 which is barely rearranged (and which lacks an endogenous CBE) thereby rendering it the most highly rearranging VH. Thus, if the engineered VH 5-1 were replaced with a human VH (and a downstream engineered CBE was included in the engineered locus), the human VH will rearrange far more frequently than it would in the absence of the CBE.

Increases in the recombination of such a VH segment can also be obtained by rendering the downstream IGCR1 non-functional. Engineering both of these modifications together tremendously increases the utilization of the targeted VH making it the most dominantly used VH. The reasons for this is that in the absence of IGCR1, RAG (the V(D)J recombination initiating enzyme) bound to a DJ_{H}recombination center can more readily find its next upstream target VH by a linear scanning mechanism during which their interaction with the RAG RC is promoted by an associated downstream CBE to promote their accessibility for rearrangement.

In the IgL (κ) locus, rendering the Cer/Sis sequence non-functional also enhance utilization of proximal Vκ segments due to a scanning mechanism similar to what occurs in the absence of IGCR1 function in the IgH locus. As demonstrated herein, a Cas9-gRNA based approach was used to delete the Sis/Cer elements of the Igk locus in a mouse v-Abl pre-B cell line that can be induced in vitro to undergo Igκ V(D)J recombination. After control and Sis/Cer deleted v-Abl pre-B cells were induced to undergo V(D)J recombination of their endogenous Igκ locus, HTGTS-based high throughput V(D)J recombination assay was used to analyze the frequency with which different endogenous Vκ segments rearranged to a Jk4 bait sequence. It was demonstrated that deletion of Cer/Sis elements substantially increased the rearrangement frequency of the proximal Vκ3-1, Vκ3-2 and Vκ3-3 segment (FIG. 15A-15C), consistent with allowing RAG scanning between the Jκ recombination center and the proximal Vκ_{S}.

Accordingly, as described herein, a target V segment (e.g., human Vκ3-20 or Vk1-33 segment), when positioned in place of the proximal mouse Vκ segments in the context of Sis/Cer deletion, will also be preferentially utilized during V(D)J recombination. Due to junctional diversification, the B cell population in this model expresses diverse repertoires of Vκ3-20 and/or Vk1-33 light chains; and, as described above, such diversity can be made even more human-like by incorporation of constitutive TdT expression in the ES cell-based model (which increased CDR 3 diversity. In a v-Abl model cell line system. deletion of Cer alone provided a similar phenotype regarding proximal Vκ rearrangement as deleting Cer/Sis indicating that deletion of Cer is sufficient to induce preferential rearrangement of proximal Vκ segments.

Furthermore, based on IgH results described elsewhere herein, adding a CBE to a proximal IgL Vκ will lead to its additionally enhanced utilization, particularly in the absence of Cer/Sis to allow unabated RAG scanning from the Jκ recombination center. Combined with the ability to replace the V segments themselves, e.g., proximal mouse VH and VL segments, with IGH and/or IgL V segments of particular interest, such modifications, when combined, will permit creation of immunoglobin repertories which comprise the VH and VL segment(s) of interest combined to diverse CDR3s at a much higher frequency than would occur naturally, a frequency that would much more approximate the frequency of these nacent antibodies (BCR) in the much larger human BCR repertoires. This discovery permits the engineering of antibodies comprising a desired VH and VL segment(s) while still allowing the antibody to participate in V(D)J recombination, somatic hypermutation, and the germinal center reaction—important processes that contribute to antibody diversity (e.g., CDR 3 diversity) and functionality. Notably, the complexity of the CDR3, which is arguably the greatest site for antigen contact diversity, would be far higher than in other existing humanized mouse models, which permits, upon immunization, the selection of a broader set of specific antibody precursors than exist in prior mouse models and the selected precursor antibody will then be further optimized by SHM of all three CDRs (including CDR3) upon further immunization and selection during the GC reaction. Thus, these methods and compositions described herein can permit the discovery of novel therapeutic antibodies and/or also can be used, to further improve specificity and/or affinity of an existing antibody.

In one aspect of any of the embodiments, described herein is a cell comprising at least one of:
a. an engineered IgH locus comprising a CBE element within the nucleic acid sequence separating the 3′ end of a target VH segment and the 5′ end of the first VH segment which is 3′ of the target VH segment; and/or
b. an engineered IgL locus comprising at least one of:
   i. a non-functional Cer/Sis sequence within the nucleic acid sequence separating the 3′ end of the 3′-most V_{L} segment and the 5′ end of a J_{L} segment; and
   ii. a CBE element within the nucleic acid sequence separating the 3′ end of a target V_{L} segment and the 5′ end of the first V_{L} segment which is 3′ of the target V_{L}segment.
      In some embodiments of any of the aspects, the CBE element is located 5′ of at least one V segment in the locus. In some embodiments of any of the aspects, the CBE element is in the same orientation as the target segment. In some embodiments of any of the aspects, the CBE element is in the inverted orientation with respect to the target segment. In some embodiments of any of the aspects, the CBE element is located 3′ of the VH recombination signal sequence of the target V segment.

In some embodiments of any of the aspects, the target V_{H} or V_{L} segment is a non-native, exogenous, or engineered segment. In some embodiments of any of the aspects, the cell is a mouse cell and the target V_{H} or V_{L} segment is a human segment. In some embodiments of any of the aspects, the cell further comprises a non-native D_{H}, J_{H}, and/or I_{L} segment. In some embodiments of any of the aspects, the non-native D_{H}, J_{H}, or I_{L} segment is a human segment. In some embodiments of any of the aspects, the human segment is from a known antibody in need of improvement of affinity or specificity. In some embodiments of any of the aspects, the human segments are highly-utilized human segments.

In some embodiments of any of the aspects, the cell is a stem cell embryonic stem cell. In some embodiments of any of the aspects, the cell is a murine cell, optionally a murine stem cell or murine embryonic stem cell.

In some embodiments of any of the aspects, the cell is heterozygous for the engineered IgH and/or IgL locus and the other IgH and/or IgL locus has been engineered to be inactive, wherein the cell will express an IgH and/or IgL chain only from the engineered IgH and/or IgL locus. In some embodiments of any of the aspects, the cell further comprises an engineered non-functional IGCR1 sequence in the IgH within the nucleic acid sequence separating the 3′ end of the 3′-most VH segment of the IgH locus and the 5′ end of a D_{H} segment of the IgH locus. In some embodiments of any of the aspects, the non-functional IGCR1 sequence comprises mutated CBE sequences; the CBE sequences of the IGCR1 sequence have been deleted; or the IGCR1 sequence has been deleted from the IgH locus.

In some embodiments of any of the aspects, the cell further comprises at least one of the following:
a. an IgL locus with human sequence;
b. a humanized IgL locus;
c. a human IgL locus;
d. an IgH locus with human sequence;
e. a humanized IgH locus; and
f. a human IgH locus.
   In some embodiments of any of the aspects, the cell further comprises at least one of the following:
a. the engineered IgH locus further engineered to comprise only one VH segment (e.g., one human VH segment);
b. the engineered IgL locus further engineered to comprise only one V_{L} segment (e.g., one human V_{L} segment);
c. the IgL locus engineered to comprise one J_{L} segment;
d. an IgH locus engineered to comprise one J_{H} segment; and
e. an IgH locus engineered to comprise one D_{H} segment.

In some embodiments of any of the aspects, the cell further comprises a mutation capable of activating, inactivating or modifying genes lead to increased GC antibody maturation responses. In some embodiments of any of the aspects, the cell further comprises a cassette targeting sequence in the target segment, which permits the replacement of the target segment. In some embodiments of any of the aspects, the cassette targeting sequence is selected from the group consisting of: an I-SceI meganuclease site; a Cas9/CRISPR target sequence; a Talen target sequence or a recombinase-mediated cassette exchange system. In some embodiments of any of the aspects, the cell further comprises an exogenous nucleic acid sequence encoding TdT. In some embodiments of any of the aspects, a promoter is operably linked to the sequence encoding TdT.

In one aspect of any of the embodiments, described herein is a genetically engineered mammal comprising a cell as described herein. In one aspect of any of the embodiments, described herein is a genetically engineered mammal consisting essentially of cells as described herein. In one aspect of any of the embodiments, described herein is a genetically engineered mammal consisting of cells as described herein. In one aspect of any of the embodiments, described herein is a chimeric genetically engineered mammal comprising two populations of cells,
a first population comprising cells which are V(D)J recombination-defective; and
a second population comprising engineered cells as described herein.
   In some embodiments of any of the aspects, the V(D)J recombination-defective cells are RAG2^{-/-} cells. In some embodiments of any of the aspects, the mammal is a mouse.

In one aspect of any of the embodiments, described herein is a genetically engineered mammal comprising a population of cells comprising at least one of:
a. an engineered IgH locus comprising at least one of:
   i. a CBE element within the nucleic acid sequence separating the 3′ end of a target V_{H} segment and the 5′ end of the first VH segment which is 3′ of the target V_{H}segment;
   ii. an engineered non-functional IGCR1 sequence in the IgH locus within the nucleic acid sequence separating the 3′ end of the 3′-most VH segment of the IgH locus and the 5′ end of a D_{H} segment of the IgH locus; and/or
b. an engineered IgL locus comprising at least one of:
   i. a non-functional Cer/Sis sequence within the nucleic acid sequence separating the 3′ end of the 3′-most V_{L} segment and the 5′ end of a J_{L} segment; and
   ii. a CBE element within the nucleic acid sequence separating the 3′ end of a target V_{L} segment and the 5′ end of the first V_{L} segment which is 3′ of the target V_{L}segment;
   whereby V(D)J recombination in the mammal predominantly utilizes the target V_{H} segment and the target V_{L} segment and/or V(D)J recombination in the mammal predominantly utilizes the target VH segment and has enhanced utilization of the target V_{L} segment.

In some embodiments of any of the aspects, the target VH segment and/or the target V_{L}segment are human V segments. In some embodiments of any of the aspects, the IgH locus is further engineered to comprise one target D segment and/or one target J_{H} segment. In some embodiments of any of the aspects, the IgL locus is further engineered to comprise one target J_{L} segment. In some embodiments of any of the aspects, the D segment, J_{H} segment, and/or J_{L} segment are human segments. In some embodiments of any of the aspects, the human segments are from a known antibody in need of improvement of affinity or specificity. In some embodiments of any of the aspects, the human segments are highly-utilized human segments. In some embodiments of any of the aspects, the cell is heterozygous for the engineered IgH and/or IgL locus and the other IgH and/or IgL locus has been engineered to be inactive, wherein the cell will express an IgH and/or IgL chain only from the engineered IgH and/or IgL locus. In some embodiments of any of the aspects, the CBE element is located 5′ of at least one V segment in the locus. In some embodiments of any of the aspects, the CBE element is in the same orientation as the target segment. In some embodiments of any of the aspects, the CBE element is in the inverted orientation with respect to the target segment. In some embodiments of any of the aspects, the CBE element is located 3′ of the VH recombination signal sequence of the target V segment. In some embodiments of any of the aspects, the cell or mammal further comprises a mutation capable of activating, inactivating or modifying genes lead to increased GC antibody maturation responses. In some embodiments of any of the aspects, the cell or mammal further comprises an exogenous nucleic acid sequence encoding TdT. In some embodiments of any of the aspects, a promoter is operably linked to the sequence encoding TdT.

In some embodiments of any of the aspects, the mammal is a mouse or the cell is a mouse cell.

In one aspect of any of the embodiments, described herein is a set of at least two mammals, wherein each mammal is a mammal as described herein, the first mammal comprising a first target V_{H} segment and/or a first target V_{L} segment and each further mammal comprising a further target V_{H} segment and/or a further target V_{L} segment. In some embodiments of any of the aspects, each mammal comprises a human target V_{H} segment and a human target V_{L} segment.

In one aspect of any of the embodiments, described herein is a method of making an antibody, the method comprising the steps of: injecting a mouse blastocyst with a cell as described herein, wherein the cell is a mouse embryonic stem cell; implanting the mouse blastocyst into a female mouse under conditions suitable to allow maturation of the blastocyst into a genetically engineered mouse; and isolating
1) an antibody; or
2) a cell producing an antibody
   from the genetically engineered mouse. In some embodiments of any of the aspects, the method further comprises a step of immunizing the genetically engineered mouse with a desired target antigen before the isolating step. In some embodiments of any of the aspects, the method further comprises a step of producing a monoclonal antibody from at least one cell of the genetically engineered mouse. In some embodiments of any of the aspects, the one or more target segments comprise a non-native V_{L} or V_{H}segment. In some embodiments of any of the aspects, one or more target segments comprise a non-native V_{L} or V_{H} segment of a known antibody, whereby the known antibody is optimized.

In one aspect of any of the embodiments, described herein is a method of making an antibody, the method comprising the steps of: isolating an antibody comprising the one or more target segments from a mammal or set of mammals described herein, or isolating a cell expressing an antibody comprising the one or more target segments from the mammal or set of mammals described herein. In some embodiments of any of the aspects, the method further comprises a step of immunizing the genetically engineered mammal or set of mammals with a desired target antigen before the isolating step.

In one aspect of any of the embodiments, described herein is a method of making an antibody which is specific for a desired antigen, the method comprising the steps of:
a) injecting a mouse blastocyst with a cell as described herein, wherein the cell is a mouse embryonic stem cell and implanting the mouse blastocyst into a female mouse under conditions suitable to allow maturation of the blastocyst into a genetically engineered mouse or do by RDBC;
b) immunizing the genetically engineered mouse with the antigen; and
c) isolating
   1) an antibody specific for the antigen; or
   2) a cell producing an antibody specific for the antigen
from the genetically engineered mouse.
   In one aspect of any of the embodiments, described herein is a method of making an antibody which is specific for an antigen, the method comprising the steps of:
a) immunizing a mammal or a set of mammals as described herein with the antigen; and
b) isolating
   1) an antibody specific for the antigen; or
   2) a cell producing an antibody specific for the antigen
from the mammal or mammals.
   In some embodiments of any of the aspects, the method further comprises a step of producing a monoclonal antibody from at least one cell of the genetically engineered mouse or mammal.

In one aspect of any of the embodiments, described herein is an antibody produced by any one of the methods described herein.

In some embodiments of any of the aspects, the antibody is an optimized antibody. In some embodiments of any of the aspects, the antibody is a humanized antibody.

In one aspect of any of the embodiments, described herein is a method of identifying a candidate antigen as an antigen that activates a B cell population comprising a V_{H} or V_{L} segment of interest, the method comprising: immunizing a mammal as described herein, engineered such that a majority of the mammal's peripheral B cells express the V_{H} or V_{L} segment of interest, with the antigen; measuring B cell activation in the mammal; and identifying the candidate antigen as an activator of a B cell population comprising the V_{H} or V_{L} segment of interest if the B cell activation in the mammal is increased relative to a reference level. In some embodiments of any of the aspects, an increase in B cell activation is an increase in the somatic hypermutation status of the Ig variable region; an increase in the affinity of mature antibodies for the antigen; or an increase in the specificity of mature antibodies for the antigen.

### BRIEF DESCRIPTION OF THE DRAWINGS

FIGS. 1A-ID demonstrate that VH81X-CBE Greatly Enhances VH81X Utilization in Primary Pro-B Cells. FIG. 1A depicts a schematic of the murine Igh locus showing proximal VHs, Ds, JHs, CH exons and regulatory elements (not to scale). Light and dark grey bars represent members of the IGHV5 (VH7183) and IGHV2 (VHQ52) families, respectively. Triangles represent position and orientation of CTCF-binding elements (CBEs). Arrow denotes position of the JH4 coding end bait primer used to generate HTGTS-Rep-Seq libraries. FIG. 1B depicts the sequence of VH81X-RSS (bold) followed by WT (dashed box) or scrambled (solid box) VH81X-CBE. FIG. 1B discloses SEQ ID NOS 51-52, respectively, in order of appearance. FIG. 1C depicts relative VH utilization+SD Standard Deviation (SD) in BM pro-B cells from WT (top) or VH81X-CBEscr/scr (bottom) mice. FIG. 1D depicts average utilization frequencies (left axis) and % usage (right axis) of indicated proximal VH segments+SD. For analysis, each library was normalized to 10,000 VDJH junctions. p values were calculated using unpaired, two-tailed Student's t-test, ns indicates p≥0.05, * p≤0.05, ** p≤0.01 and *** p≤0.001. For analysis, each library was normalized to 10,000 VDJH junctions.

FIGS. 2A-2G demonstrate that VH81X-CBE Enhances VH81X Utilization in DJH Rearranged v-Abl Pro-B Lines. FIG. 2A depicts a schematic representation of the two murine Igh alleles in DJH rearranged v-Abl pro-B cell line (not to scale). One allele (top) harbors a non-productive VDJH rearrangement involving a distal VHJ558 (VH1-2P) which deletes the proximal VH domain and is inert for V(D)J recombination. The other allele (bottom) harbors a DHFL16.1 to JH4 rearrangement (DJH allele) that actively undergoes VH to DJH recombination upon RAG induction via G1 arrest. This DHFL16.1JH4 line served as the parent WT line and was used for all subsequent genetic manipulations. In FIG. 2B top line shows the sequence of WT VH81X-CBE while the bottom line shows VH81X-CBE deletion. FIG. 2B discloses SEQ ID NOS 53-54, respectively, in order of appearance. FIG. 2C depicts average utilization frequencies (left axis) or % usage (right axis)±SD of indicated proximal VHs in WT and VH81X-CBEdel v-Abl pro-B lines; libraries were normalized to 3,500 VDJH junctions. As the WT line used for this experiment was the parent of all subsequent VH-CBE mutant lines, we generated WT repeats at several points over the course of these experiments and used the average data, which were highly reproducible, for this and subsequent panels showing comparisons of mutants with WT controls (see STAR Methods for details). FIG. 2D depicts a schematic of the 101-kb intergenic deletion extending from 302 bp downstream of VH81X-CBE to about 400 bp upstream of the DHFL16.1JH4 RC in the WT DHFL16.1JH4 v-Abl line and its VH81X-CBEdel derivative. FIG. 2E depicts average utilization frequencies (left axis) or % usage (right axis)±SD of indicated proximal VHs in Intergenicdel and Intergenicdel VH81X-CBEdel v-Abl lines; libraries were normalized to 100,000 VDJH junctions. FIG. 2F depicts the sequence of WT and VH81X-CBE inversion mutation. FIG. 2F discloses SEQ ID NOS 55-56, respectively, in order of appearance. FIG. 2G depicts average utilization frequencies (left axis) or % usage (right axis)±SD of the indicated proximal VHs in DHFL16.1JH4 WT and VH81X-CBEinv v-Abl lines; libraries were normalized to 3,500 VDJH junctions. Statistical analyses were performed as in FIG. 1A-1D.

FIGS. 3A-3C demonstrate that VH81X-CBE Promotes Interactions of its Flanking VH with the DJHRC. FIG. 3A depicts a schematic representation of the 3C-HTGTS method for studying chromosomal looping interactions of a bait region of interest with the rest of Igh locus (see text and STAR Methods for details). FIG. 3B depicts a schematic of the NlaIII restriction fragment (indicated by a asterisk) and the relative positions of the biotinylated (arrow with dotted tail) and nested (regular arrow) PCR primers used for 3C-HTGTS from VH81X bait in FIG. 3C. In FIG. 3C, top panel is a schematic representation of chromosome interactions of VH81X-CBE containing NlaIII fragment with other Igh locales. Bottom two panels are 3C-HTGTS profiles of Rag2−/− derivatives of control, VH81X-CBEdel and VH81X-CBEinv DHFL16.1JH4 v-Abl lines using VH81X-CBE locale as bait. Owing to a DHFL16.1 to JH4 rearrangement in the lines, the region spanning IGCR1, DJH substrate and iEμ appears as a broad interaction peak. As v-Abl lines lack locus contraction, we detected few substantial interactions with the upstream Igh locus beyond the most proximal VHs. Two independent data sets are shown from libraries normalized to 105,638 total junctions.

FIGS. 4A-4D demonstrate that V(D)J Recombination of VH2-2 Is is Critically Dependent on its Flanking CBE. FIG. 4A depicts the sequence of WT VH2-2-CBE and its scrambled mutation. FIG. 4A discloses SEQ ID NOS 57-58, respectively, in order of appearance. FIG. 4B depicts average utilization frequencies (left axis) or % usage (right axis)±SD of indicated proximal VHs in WT and VH2-2-CBEscr v-Abl lines. Each library was normalized to 3,500 VDJH junctions. Statistical analyses were performed as in FIGS. 1A-1D. FIG. 4C depicts an illustration of NlaIII restriction fragment (asterisk) and relative positions of biotinylated (arrow with dotted tail) and nested (regular arrow) primers used for 3C-HTGTS analyses in FIG. 4D. Due to repetitive sequences in the restriction fragment that harbors VH2-2-CBE, the downstream flanking restriction fragment was used as bait. FIG. 4D depicts representative 3C-HTGTS interaction profiles of VH2-2 locale (asterisk) in Rag2−/− control and VH2-2-CBEscr v-Abl lines, plotted from libraries normalized to 84,578 total junctions.

FIGS. 5A-5D demonstrate that VH81X-CBE is Required for Dominant VH81X Usage in the Absence of IGCR1. FIG. 5A depicts a schematic of 4.1 kb IGCR1 deletion. FIG. 5B depicts average utilization frequencies (left axis) or % usage (right axis)±SD of proximal VHs in IGCR1del and IGCR1del VH81X-CBEdel v-Abl lines. Each library was normalized to 100,000 VDJH junctions. Statistical analyses were performed as in FIGS. 1A-1D. FIG. 5C depicts representative 3C-HTGTS interaction profiles of VH81X bait (asterisk) in Rag2−/− control, IGCR1del and IGCR1del VH81X-CBEdel DHFL16.1JH4 v-Abl lines performed using the strategy shown in FIG. 3B, plotted from libraries normalized to 106,700 total junctions. Bottom panel shows a zoom-in of the region extending from upstream of IGCR1 to downstream of Cδ exons. Rectangles marked with “A” indicate the IGCR1 region deleted in the IGCR1del and IGCR1del VH81X-CBEdel IGCR1del lines. FIG. 4D depicts representative 3C-HTGTS interaction profiles of iEμ bait (asterisk) in Rag2−/− v-Abl DJH lines of the indicated genotypes following NlaIII digest using the strategy shown in FIG. 12D. Each library was normalized to 273,547 total junctions. Bottom panel shows a zoom-in of the proximal VH region.

FIGS. 6A-6D demonstrate that restoration of a CBE Converts VH5-1 into the Most Highly Rearranging VH. FIG. 6A depicts a schematic showing the sequence of VH5-1-RSS and its downstream non-functional, “vestigial” CBE. The box highlights the CpG island that is methylated in normal pro-B cells. Bottom sequence shows the four nucleotides mutated (highlighted in solid unshaded boxes) to eliminate the CpG island and restore consensus CBE sequence. Two additional nucleotides were mutated just downstream of the CBE to generate a BglII site for screening. FIG. 6A discloses SEQ ID NOS 59-61, respectively, in order of appearance. FIG. 6B depicts average utilization frequencies (left axis) or % usage (right axis)±SD of the indicated proximal VHs in WT and VH5-1-CBEins v-Abl lines. Each library was normalized to 3,500 VDJH junctions. Statistical analyses were performed as in FIGS. 1A-1D. FIG. 6C depicts an illustration of the MseI restriction fragment (asterisk) and the relative positions of biotinylated (arrow with dotted tail) and nested (regular arrow) primers used for 3C-HTGTS analyses in FIG. 6D. FIG. 6D depicts representative 3C-HTGTS interaction profiles of the VH5-1 locale (asterisk) in Rag2−/− control and VH5-1-CBEins v-Abl lines, plotted from libraries normalized to 37,856 total junctions.

FIGS. 7A-7F depict a model for RAG Chromatin Scanning via Loop Extrusion. Shown is a working model for potential roles of VH-associated CBEs during RAG scanning over chromatin. Numerous variations of the model are conceivable. FIG. 7A demonstrates that from its location in the initiating RC, RAG linearly scans cohesin-mediated extrusion loops proceeding through Ds, to allow their utilization; but is largely impeded further upstream by the IGCR1 anchor. After formation of a DJHRC, residual lower level scanning of upstream sequences beyond the IGCR1 impediment allows the most proximal VH-CBEs to mediate direct association with the DJHRC enhancing utilization of their associated VH. VHs further upstream likely access the DJHRC by diffusion with proximal CBEs also enhancing DJHRC interactions and flanking VH utilization. FIG. 7B demonstrates that in the absence of IGCR1, loop extrusion progresses upstream allowing RAG to scan the most proximal VHs where associated CBEs promote DJHRC interaction, accessibility, and dominant over-utilization in V(D)J joins. Utilization is most robust for proximal VH81X, which provides the first VH-CBE encountered during linear scanning. VH5-1 is bypassed due to lack of a CBE. Scanning can sometimes bypass VH81X-CBE and continues to the first few upstream VHs, with their CBEs similarly promoting utilization. FIG. 7C demonstrates that if both IGCR1 and the VH81X-CBE are mutated, loop-extrusion continues unabated to the VH2-2-CBE and to progressively lesser extents to immediately upstream VH-CBEs. (FIGS. 7D-7F) CBEs not directly flanking distal VHs theoretically also may augment VH utilization. FIG. 7D demonstrates that a distal VH locus CBE associates strongly with chromatin or associated factors (e.g. CTCF/Cohesin) at the DJHRC. In FIG. 7E, cohesin rings load near this DJHRC-associated distal VH locus CBE and initiate loop extrusion. In FIG. 7F, loop-extrusion allows RAG to scan downstream (or upstream, not illustrated) VHs lacking directly associated CBEs from the DJHRC where the active/transcribed chromatin in which they lie facilitates access for V(D)J recombination,

FIGS. 8A-8E demonstrate that the Vast Majority of Functional Igh VHs Harbor a CBE in their Vicinity. FIG. 8A demonstrates that the approximately 2.4 Mb C57BL/6 mouse VH region divided in to four domains (Choi et al., 2013) from most JH-proximal to most JH-distal: about 0.31 Mb proximal 7183/Q52 domain harboring 18 members of the IGHV5 and IGHV2 families, about 0.56 Mb domain harboring 31 members belonging to 10 different middle VH families, about 0.53 Mb J558 domain harboring 34 IGHV1 family members, 2 IGHV10 members and 1 each of IGHV8 and IGHV15 families, and the most distal about 1 Mb J558/3609 domain harboring 32 IGHV1 members interspersed with 8 IGHV8 family members are indicated. These VH numbers reflect only the VHs that undergo V(D)J recombination at detectable frequency. FIGS. 8B-8E depict VH segments from the four respective VH domains arranged in order of their utilization frequency from highest (left) to lowest (right). % VH usage was calculated from total out-of-frame VDJH junctions obtained from B220+CD43highIgM-pro-B cells derived from 4-6 weeks old mice after normalizing each individual library to 3,564 out-of-frame VDJH junctions, n=3 (data extracted from Lin et al., 2016). Data represent mean±SD. Only out-of-frame junctions were analyzed to examine primary rearrangement frequencies with minimum effect of cellular selection on IgH repertoires. White bars indicate VHs that show a CTCF ChIP-seq peak in Rag2−/− pro-B cells (Choi et al., 2013) within 10 kb of their RSS and without the presence of an intervening functional VH segment between the VH and CTCF peak in question. The grey bars represent VHs that do not fit this criterion. Asterisks on top of white bars indicate the relative distance of the CTCF peak from the VH-RSS: *CTCF ChIP-seq peak within 100 bps, **within 5 kb and ***within 10 kb of the VH-RSS. VH segments that did not show CTCF binding within 10 kb of their RSS but contributed to >0.5% of all rearrangements frequently have a nearby Pax5 or YY1 ChIP-seq peak in Rag2−/− pro-B cells (Revilla-I-Domingo et al., 2012; Medvedovic et al., 2013). These sites, which may theoretically serve overlapping functions to CBE interactions in the model in FIGS. 7A-7E, are shown on top of grey bars wherever present.

FIGS. 9A-9F demonstrate the generation of VH81X-CBEscr/scr Mice. FIG. 9A depicts an electrophoretic mobility gel shift assay (EMSA) to confirm loss of CTCF binding to a scrambled VH81X-CBE sequence that was subsequently used to generate VH81X-CBEscr/scr mice. Addition of anti-CTCF antibody results in a super-shift indicating binding of CTCF to the WT VH81X-CBE sequence (shown in red above). Addition of 20- or even 200-fold molar excess of unlabeled scrambled VH81X-CBE oligo could not compete with the WT oligo for CTCF binding. FIG. 9A discloses SEQ ID NOS 62-63, respectively, in order of appearance. FIG. 9B depicts a schematic of the targeting strategy used to generate 129SV ES cells harboring the VH81X-CBEscr mutation. Indicated arrows indicate position of PCR primers used to confirm CBE mutation. FIGS. 9C, 9D, and 9F depict Southern blot confirmation of the targeted ES cells. FIG. 9E demonstrates that VH81X-CBEscr mutation was confirmed by PCR-amplifying the region flanking VH81X-CBE followed by restriction digestion with NotI.

FIGS. 10A-10C demonstrate VH Usage in v-Abl DHFL16.1JH4 Lines. Depicted are utilization frequencies of VHs across the entire Igh locus in WT parental DHFL16.1JH4 line and its mutant derivatives as determined by HTGTS-Rep-Seq using a JH4 coding end bait primer. Analyses were performed after arresting cells in G1 with STI-571 treatment for four days. Data represent average rearrangement frequencies±SD obtained after normalizing each individual library to 3,500 (FIGS. 10A, 10C) and 100,000 (FIG. 10B) VDJH junctions. In addition to the 101-kb intergenic deletion v-Abl DHFL16.1JH4 lines (FIG. 2D) analyzed in FIG. 10B and FIG. 2E, we made partial deletions encompassing either the DJH-proximal 50 kb region or the DJH-distal 54 kb region in VH81X-CBEdel and VH81X-CBEdel IGCR1del backgrounds, respectively; their rearrangement profiles looked indistinguishable from those of the VH81X-CBEdel IGCR1del lines (data not shown). We note that comparative 3C-HTGTS studies of primary RAG2-deficient pro-B cells and v-Abl pro-B lines indicated similar interactions among sequences in the region between IGCR1 and 3′CBEs, but lack of interactions with VH locus sequences in RAG2 deficient v-Abl pro-B lines other than with the most proximal VHs (Ba, Z., Lin, S., and Alt, F. W, unpublished data). Together with lack of distal VH V(D)J recombination shown in this figure, these findings indicate that Igh is not contracted in v-Abl pro-B lines.

FIGS. 11A-11D demonstrate VH Usage and 3C-HTGTS Profiles of Control, VH2-2-CBEscr and VH5-1-CBEins v-Abl DHFL16.1JH4 Lines. FIGS. 11A and 11C depict rearrangement frequencies of VHs across the entire Igh locus in VH2-2scr (FIG. 11A) and VH5-1ins (FIG. 11C) DHFL16.1JH4 v-Abl lines relative to WT control as determined by HTGTS-Rep-Seq using a JH4 coding end bait primer. Analyses were performed after arresting cells in G1 with STI-571 treatment for four days. Data represent average rearrangement frequencies+SD obtained after normalizing each individual library to 3,500 VDJH junctions. FIGS. 11B and 11D depict additional 3C-HTGTS repeats showing chromatin interaction profiles of the VH2-2 (FIG. 11B) and VH5-1 (FIG. 11D) locales (asterisks), in Rag2−/− control and mutant DHFL16.1JH4 v-Abl pro-B cell lines using bait primers shown in FIGS. 4C and 6C, respectively. Data were plotted from libraries normalized to 84,587 and 37,856 total junctions in (FIG. 11B) and (11D), respectively.

FIGS. 12A-12D depict interaction profiles of VH81X and iEμ in DHFL16.1JH4 v-Abl Lines. FIG. 12A depicts average frequency of proximal VH utilization in WT and IGCR1del DHFL16.1JH4 v-Abl lines as determined by HTGTS-Rep-Seq using a JH4 coding end bait primer after four days of G1 arrest. Data represent the average utilization frequencies (left axis) or % usage (right axis)±SD obtained after normalizing each individual library to 120,000 aligned reads which include all DHFL16.1JH4 reads as well as VH to DHFL16.1JH4 junctions. p values were calculated using unpaired, two-tailed Student's t-test, ns indicates p>0.05, * p≤0.05, ** p≤0.01 and *** p≤0.001. FIG. 12B depicts rearrangement frequencies of VHs across the entire Igh locus in IGCR1del (top) and IGCR1del VH81X-CBEdel (bottom) DHFL16.1JH4 v-Abl lines as determined by HTGTS-Rep-Seq using a JH4 coding end bait primer after four days of G1 arrest. Data represent average rearrangement frequencies+SD obtained after normalizing each individual library to 100,000 VDJH junctions. FIG. 12C depicts additional 3C-HTGTS repeat showing chromatin interaction profiles of the VH81X locale (asterisk) in Rag2−/− control, IGCR1del and IGCR1del VH81X-CBEdel DHFL16.1JH4 v-Abl lines performed using the baiting strategy shown in FIG. 3B. Data were plotted from libraries normalized to 106,700 total junctions. Bottom panel shows a zoom-in of the region extending from upstream of IGCR1 to downstream of Cδ. FIG. 12D depicts additional 3C-HTGTS repeat showing chromatin interaction profiles of the iEμ locale (asterisk) in Rag2−/− control, IGCR1del and IGCR1del VH81X-CBEdel DHFL16.1JH4 v-Abl lines using the baiting strategy shown on the right. Data were plotted from libraries normalized to 273,547 total junctions. Bottom panel shows zoom-in of the proximal VH region.

FIGS. 13A-13B depict chromosomal interaction interaction profiles Profiles of iEμ and DHQ52-JH1 locales Locales in unrearranged Unrearranged v-Abl proPro-B linesLines. FIG. 13A depicts representative 3C-HTGTS interaction profiles of the iEμ fragment (asterisk) in Rag2−/− derivatives of unrearranged WT, IGCR1del/del and IGCR1del/del VH81X-CBEscr/scr IGCR1del/del v-Abl lines using the baiting strategy shown in FIG. 12D. Data were plotted from libraries normalized to 215,280 total junctions. Bottom panel shows zoom-in of the proximal VH region. FIG. 13B depicts a comparison of 3C-HTGTS interaction profiles in Rag2−/− IGCR1del/del v-Abl lines from iEμ and DHQ52-JH1 baits within the RC, plotted from libraries normalized to 215,280 total junctions. The Igh locale on chr12 from 114,400,000-114,893,000 nucleotides of the AJ851868/mm9 hybrid genome is shown. The baiting strategy used for DHQ52-JH1 bait is shown on the right. Both iEμ and DHQ52-JH1 baits revealed an additional DHST4.1 interaction peak in these v-Abl lines that harbor unrearranged (germline configuration) Igh loci.

FIGS. 14A-14C depict VH Usage and 3C-HTGTS profiles of IGCR1del and IGCR1del VH5-1-CBEins v-Abl DHFL16.1JH4 lines. FIG. 14A depicts utilization frequencies of VHs across the entire Igh locus in IGCR1del (top) and IGCR1del VH5-1-CBEins (bottom) DHFL16.1JH4 v-Abl lines as determined by HTGTS-Rep-Seq using a JH4 coding end bait primer after four days of G1 arrest. VH81X and VH5-1 utilization bars in top and bottom panels are highlighted with arrows. Data represent average utilization frequencies±SD obtained after normalizing each individual library to 100,000 VDJH junctions. As the IGCR1del, IGCR1del VH81X-CBEdel and IGCR1del VH5-1-CBEins lines were all derived from the same ancestral DHFL16.1JH4 line, we generated IGCR1del repeats at several points during comparative analyses with IGCR1del VH81X-CBEdel or IGCR1del VH5-1-CBEins lines and have shown the average IGCR1del data here as well as in FIGS. 6B, 5B, 12A and 12B. FIG. 14B depicts average utilization frequencies (left axis) or % usage (right axis)±SD of the indicated proximal VHs (boxed in FIG. 14A). FIG. 14C depicts representative 3C-HTGTS interaction profiles of the iEμ locale (asterisk) in Rag2−/− control, IGCR1del and IGCR1del VH5-1-CBEins DHFL16.1JH4 v-Abl lines performed using the baiting strategy shown in FIG. 12D. Data were plotted from libraries normalized to 197,174 total junctions. Bottom panel shows zoom-in of the proximal VH region. Two independent repeats are shown for the Rag2−/− IGCR1del VH5-1-CBEins background.

FIGS. 15A-15B demonstrate the increased utilization of proximal Vk segments in the context of Cer/Sis deletion. FIG. 15A is a diagram illustrating the mouse Igk locus. Darker grey rectangles represent Vk segments that can be joined to Jk segments through deletional recombination, whereas lighter grey rectangles represent Vk segments that can be joined to Jk segments through inversional recombination. The plots below the diagram show Vk utilization as measured by our HTGTS method. The height of each bar represents rearrangement frequency of the indicated Vk segment. The analysis shows that deletion of Cer/Sis dramatically increases the rearrangement frequencies of the Jk-proximal Vk segments (compare Cer−/−Sis−/− with Cer+/+Sis+/+ in the area shaded with grey). FIG. 15B depicts a schematic panel zoomed in on the region from Jk to the proximal Vk segments. The histogram displays the number of sequence reads that correspond to rearrangements of individual Vk segments. Data show a major increase in rearrangement frequencies of Jk-proximal Vk segments in the absence (dark grey bars) versus presence (light grey bars) of Cer/Sis elements. The findings in this figure are consistent with RAG chromatin scanning upstream to proximal Vκs in the absence Cer/Sis, which by extension to our IgH VH CBE data shown above suggests that adding a CBE to the proximal Vκs (which lack endogenous CBEs) should greatly increase their utilization in the of Cer/Sis.

FIGS. 16-19 depict schematics of the models described in Example 5. FIG. 18 depicts a diagram of the conditional expression strategy to express an antibody in mature B cells. FIG. 19 depicts a diagram of the conditional expression to express an antibody in GC B cells.

FIG. 20 depicts graphs of HTGTS-Rep-seq of WT mouse IgM+ splenic B cells or human PBMCs using a mouse or human Jk1 bait primer. Total in-frame VJκ exons containing perfect alignments to a germline Vic sequence were used for analyses. N=1 for both mouse and human samples. Shown is the number of P/N nucleotides observed at VκJκ junctions in mouse (left) or human (right) samples, which reveals that 5% of mouse non-productive VJκ exons contain P/N nucleotides, while nearly 50% of human VJκ exons contain P/N nucleotides.

FIGS. 21A-21C. FIG. 21A demonstrates that the VRC26UCA heavy chain expression cassette, for either conditional or constitutive expression, was integrated at the JH locus of IgH^{a} allele of an Fl ES cell line. FIG. 21B demonstrates that FACS analysis of splenic B cells expressing IgM^{a} or IgM^{b}. In conditional expression model, IgM^{a+} B cells express either VRC26UCA heavy chain or the driver heavy chain. In constitutive expression model, deletion of VRC26UCA expression cassette via VH replacement allows rearrangement of the intact mouse IgHb allele and expression of IgMb. FIG. 21C depicts experiments in which single splenic B cells were sorted into 96 well plates and the VRC26UCA heavy chain transcript amplified from each single B cell. Images in this panel show results of the single-cell RT-PCR analysis. In the conditional expression model, approximately 50% of B cells express VRC26UCA heavy chain, whereas no VRC26UCA positive B cells were detectable among 96 sorted splenic B cells from the constitutive expression model.

### DETAILED DESCRIPTION

Provided herein are methods and compositions that permit a user to direct V(D)J recombination to utilize specific V segments of an Ig locus. Such methods can be utilized with wild-type V segments to generate an antibody repertoire that more frequently uses a particular V segment(s) and/or combined with additional modifications of the Ig locus in order to direct antibody repertoire development to use a non-native V segment. Three different types of Ig locus modifications are described herein, and each type can be utilized independently or in any combination with the other modification types. Additionally, the technology described herein can be combined with the IgH locus modifications described in US Patent Publication 2016/0374320; which is incorporated by reference herein in its entirety.

In one aspect of any of the embodiments, described herein is a cell comprising at least one of: a) an engineered IgH locus comprising a CBE element within the nucleic acid sequence separating the 3′ end of a target VH segment and the 5′ end of the first VH segment which is 3′ of the target VH segment; and/or b) an engineered IgL locus comprising at least one of: i) a non-functional Cer/Sis sequence within the nucleic acid sequence separating the 3′ end of the 3′-most V_{L} segment and the 5′ end of a J_{L} segment; and ii) a CBE element within the nucleic acid sequence separating the 3′ end of a target V_{L} segment and the 5′ end of the first V_{L} segment which is 3′ of the target V_{L} segment. In some embodiments of any of the aspects, the CBE element can be located downstream of the RSS which flanks the 3′ end of the target VH segment. In one aspect of any of the embodiments, described herein is a cell comprising at least one of: a) an engineered IgH locus comprising a CBE element within the nucleic acid sequence separating the 5′ end of a target VH segment and the 3′ end of the first VH segment which is proximal to the target V_{H}segment; and/or b) an engineered IgL locus comprising at least one of: i) a non-functional Cer/Sis sequence within the nucleic acid sequence separating the 3′ end of the 3′-most V_{L} segment and the 5′ end of a J_{L} segment; and ii) a CBE element within the nucleic acid sequence separating the 3′ end of a target V_{L} segment and the 5′ end of the first V_{L} segment which is 3′ of the target V_{L} segment.

In one aspect of any of the embodiments, described herein is a cell comprising an engineered IgH locus comprising a CBE element within the nucleic acid sequence separating the 3′ end of a target VH segment and the 5′ end of the first VH segment which is 3′ of the target VH segment. In some embodiments of any of the aspects, the CBE element can be located downstream of the RSS which flanks the 3′ end of the target V_{H} segment. In one aspect of any of the embodiments, described herein is a cell comprising an engineered IgH locus comprising a CBE element within the nucleic acid sequence separating the 5′ end of a target V_{H} segment and the 3′ end of the first V_{H} segment which is proximal to the target V_{H} segment.

In one aspect of any of the embodiments, described herein is a cell an engineered IgL locus comprising a non-functional Cer/Sis sequence within the nucleic acid sequence separating the 3′ end of the 3′-most V_{L} segment and the 5′ end of a J_{L} segment. In one aspect of any of the embodiments, described herein is a cell comprising an engineered IgL locus comprising a CBE element within the nucleic acid sequence separating the 3′ end of a target V_{L} segment and the 5′ end of the first V_{L} segment which is 3′ of the target V_{L} segment. In one aspect of any of the embodiments, described herein is a cell an engineered IgL locus comprising: i) a non-functional Cer/Sis sequence within the nucleic acid sequence separating the 3′ end of the 3′-most V_{L} segment and the 5′ end of a J_{L} segment; and ii) a CBE element within the nucleic acid sequence separating the 3′ end of a target V_{L} segment and the 5′ end of the first V_{L} segment which is 3′ of the target V_{L} segment.

In one aspect of any of the embodiments, described herein is a cell comprising: a) an engineered IgH locus comprising a CBE element within the nucleic acid sequence separating the 3′ end of a target V_{H} segment and the 5′ end of the first V_{H} segment which is 3′ of the target V_{H} segment; and b) an engineered IgL locus comprising: i) a non-functional Cer/Sis sequence within the nucleic acid sequence separating the 3′ end of the 3′-most V_{L} segment and the 5′ end of a J_{L} segment; and ii) a CBE element within the nucleic acid sequence separating the 3′ end of a target V_{L} segment and the 5′ end of the first V_{L} segment which is 3′ of the target V_{L} segment.

In one aspect of any of the embodiments, described herein is a cell comprising a) an engineered IgH locus comprising a CBE element within the nucleic acid sequence separating the 3′ end of a target V_{H} segment and the 5′ end of the first V_{H} segment which is 3′ of the target V_{H} segment; and b) an engineered IgL locus comprising at least one of: i) a non-functional Cer/Sis sequence within the nucleic acid sequence separating the 3′ end of the 3′-most V_{L} segment and the 5′ end of a J_{L} segment; and ii) a CBE element within the nucleic acid sequence separating the 3′ end of a target V_{L} segment and the 5′ end of the first V_{L} segment which is 3′ of the target V_{L} segment. In one aspect of any of the embodiments, described herein is a cell comprising a) an engineered IgH locus comprising a CBE element within the nucleic acid sequence separating the 3′ end of a target V_{H} segment and the 5′ end of the first V_{H} segment which is 3′ of the target V_{H} segment; and b) an engineered IgL locus comprising i) a non-functional Cer/Sis sequence within the nucleic acid sequence separating the 3′ end of the 3′-most V_{L} segment and the 5′ end of a J_{L} segment; and ii) a CBE element within the nucleic acid sequence separating the 3′ end of a target V_{L} segment and the 5′ end of the first V_{L} segment which is 3′ of the target V_{L} segment. In some embodiments of any of the aspects, the CBE element can be located downstream of the RSS which flanks the 3′ end of the target V_{H} segment. In one aspect of any of the embodiments, described herein is a cell comprising a) an engineered IgH locus comprising a CBE element within the nucleic acid sequence separating the 5′ end of a target V_{H} segment and the 3′ end of the first V_{H} segment which is proximal to the target V_{H} segment; and b) an engineered IgL locus comprising at least one of: i) a non-functional Cer/Sis sequence within the nucleic acid sequence separating the 3′ end of the 3′-most V_{L} segment and the 5′ end of a J_{L} segment; and ii) a CBE element within the nucleic acid sequence separating the 3′ end of a target V_{L} segment and the 5′ end of the first V_{L} segment which is 3′ of the target V_{L} segment. In one aspect of any of the embodiments, described herein is a cell comprising a) an engineered IgH locus comprising a CBE element within the nucleic acid sequence separating the 5′ end of a target V_{H} segment and the 3′ end of the first V_{H} segment which is proximal to the target V_{H} segment; and b) an engineered IgL locus comprising i) a non-functional Cer/Sis sequence within the nucleic acid sequence separating the 3′ end of the 3′-most V_{L} segment and the 5′ end of a J_{L} segment; and ii) a CBE element within the nucleic acid sequence separating the 3′ end of a target V_{L} segment and the 5′ end of the first V_{L} segment which is 3′ of the target V_{L} segment.

In some embodiments of any of the aspects, the CBE element can be located downstream of the RSS which flanks the 3′ end of the target V_{L} segment.

As used herein, the term “Ig locus” refers to a locus which either encodes, or can be recombined to encode, a polypeptide chain of an immunoglobin molecule (e.g. a BCR or antibody). The Ig locus can be an IgH locus (encoding the heavy chain of the immunoglobin molecule) or an IgL locus (encoding the light chain of the immunoglobin molecule). An IgL locus can be either an Igκ or an Igλ locus. Prior to VDJ recombination, an IgH locus comprises, from 5′ to 3′, one or more V_{H} segments, one or more D_{H} segments, and one or more J_{H} segments and multiple interspersed sequences, e.g. sequences that regulate and/or control the processes of VDJ recombination and expression. Prior to VDJ recombination, an IgL locus comprises, from 5′ to 3′, one or more V_{L} segments and one or more J_{L} segments and multiple interspersed sequences, e.g. sequences that regulate and/or control the processes of VJ recombination and expression.

As used herein, the term “V segment” refers to the variable segment of an Ig locus. As used herein, the term “D segment” refers to a diversity region segment of an Ig locus. As used herein, the term “J segment” refers to a joining region segment of an Ig locus. The segments can be further specified as being of the heavy or light chain, e.g., V_{H} segment or V_{L} segment respectively. One of skill in the art can readily identify such segments within an Ig locus or immunoglobin molecule. By way of non-limiting example, the structure of immunoglobins is discussed in Janeway et al. (eds.)(2001) Immunobiology. Fifth edition, Garland Sciences; Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917; which are incorporated by reference herein in their entireties.

During B cell development, an IgH D_{H} segment is first recombined with a J_{H} segment, physically joining them together to form a “DJ_{H} rearrangement”. A next step in B cell development recombines a VH segment with the DJ_{H} rearrangement to form a “V_{H}DJ_{H} rearrangement.” That is, a “V_{H}DJ_{H} rearrangement” or “DJ_{H} rearrangement” is a polynucleotide in which the named segments are recombined and intervening sequences found in the germline have been removed. Similarly, in a IgL locus, a V_{L} segment is recombined with a J_{L} segment, forming a V_{L}J_{L} rearrangement. Such rearrangements can be native constructs found in B cells or constructs created in vitro and optionally introduced into a cell.

A segment of an Ig gene, e.g., a V segment can be, e.g. a germline V segment, an affinity maturation intermediate, or a mature V segment. In some embodiments of any of the aspects, a germline segment can be a segment as found in the genome of a germline cell, e.g. prior to any V(D)J recombination event. In some embodiments of any of the aspects, a maturation intermediate can be a segment after at least one V(D)J recombination event but prior to the completion of the GC reaction and/or SHM. In some embodiments of any of the aspects, a mature segment can be a segment as found in a mature B-cell. A segment, as comprised by a maturation intermediate or a mature segment, is present in the cell as a VDJ rearrangement, having been recombined with a at least one other segment.

Certain segments, e.g., V segments are referred to herein as “target segments.” The target segment is the segment of its type (e.g., V_{H}, V_{L}, D, J_{H}, or J_{L}) which it is desired that the Ig locus will utilize in V(D)J recombination. It is not to be implied that the target V segment will be utilized in 100% of V(D)J recombination events, but that it will be utilized at a much higher rate than it would in the absence of the engineered modifications described herein. It also may be used at a higher rate that others of the same type (e.g. VHs or other VLs). The target segment (e.g., target V_{H} or V_{L} segment) can be a native, wild-type, non-native, exogenous, or engineered segment. In some embodiments of any of the aspects, the target segment (e.g., target V_{H} or V_{L} segment) can be from a different species than the cell, e.g., the cell can be a mouse cell and the target V_{H} or V_{L} segment can be a human segment.

As used herein, the term “native” refers to the sequence found in a particular location in the genome of a non-engineered cell and/or animal. As used herein, the term “non-native” refers to a sequence which varies from the sequence found in a particular location in the genome of a non-engineered cell and/or animal. A non-native sequence can be, e.g. a sequence from a different species or a sequence from the same species which has been moved to a non-native position in the genome. Thus, while a sequence may be “native” to a particular gene in the genome of an un-engineered cell, if it has been moved within the gene in an engineered cell, it is no longer considered native. In some embodiments of any of the aspects, a non-native sequence differs from the native sequence by, at least 5%, e.g. at least 5%, at least 10%, at least 20%, at least 30%, at least 40%, at least 50% or more.

In some embodiments of any of the aspects, the Ig locus is a mouse locus and the target V segment of the Ig locus has been engineered to comprise any V segment other than the original mouse V segment. In some embodiments of any of the aspects, the non-native V segment is a human V segment. In some embodiments of any of the aspects, the non-native V segment is a V segment from a known antibody in need of improvement of affinity, specificity, or breadth for which improvements in any or all of these properties is desired. In some embodiments of any of the aspects, the non-native V segment is a human V segment from a known antibody in need of improvement of affinity or specificity, or breath for which improvement of any or all of these or other properties is desired. In some embodiments of any of the aspects, the non-native V segment is a V segment from a known antibody. In some embodiments of any of the aspects, the non-native V segment is a human V segment from a known antibody. In some embodiments of any of the aspects, the V segment may be a commonly utilized human VH or VL segment.

While the methods and compositions described herein are suitable for use with any V segment, certain specific V segments, which may be from the germline or from a previously affinity matured antibody and thus harbor SHMs, are particularly contemplated for use in the compositions and methods described herein due to their known antigen specificities. Other V segments may be selected due to the high frequency with which the contribute to unselected antibody repertoires such as, but not limited to IGHV1-2*02, IGHV1-69, VH3-30, and VH4-59. The sequences of these V_{H} segments are known in the art, for example, IGHV1-2*02 is described by Genbank Accession No: FN550184.1 (SEQ ID NO: 1) and SEQ ID NO: 13 of International Patent Publication WO 2010/054007; and IGVH1-46 is described by Genbank Accession No: AJ347091.1 (SEQ ID NO: 2). In some embodiments of any of the aspects described herein, the V_{L} segment can be selected from the group consisting of: the frequently utilized Vκs and Vλs including but not limited to Vκ1-5, Vκ3-20, Vκ4-1, Vλ1-51, Vλ3-1, Vλ2-14.

In some embodiments of any of the aspects, the V segments can be the V segments of 2G12 bnAb or VRC42 bnAb. The V segments of 2G12 bnAb are: VH3-21, Vk1-5 and the V segments of VRC42 bnAb are: VH1-69, Vk3-20.

As used herein, “Cer/Sis sequence” refers collectively to Cer and/or Sis elements of Ig loci. Cer (contracting element for recombination) and Sis (silencer in the intervening sequence) elements are known elements of Ig genes. As used herein, “contracting element for recombination” or “Cer” refers to a region located in the IgL locus 3′ of the 3′-most native VL segment and the 5′ end of the 5′-most native JL segment and which controls VJ recombination. Cer is approximately 650 bp in length. Cer can bind CTCF and is DNaseI hypersensitive. As used herein, “silencer in the intervening sequence” or “Sis” refers to a region located in the IgL locus 3′ of the 3′-most native VL segment and the 5′ end of the 5′-most native JL segment and which controls VJ recombination. Sis is approximately 1,500 bp in length. Sis can bind CTCF and Ikaros and is also DNaseI hypersensitive. The structure of Cer and Sis are explained in more detail, e.g., in Xiang et al. J Immunol 190:1819-1826(2013); Liu et al. J Biol Chem 277:32640-32649 (2002); and Liue et al. Immunity. 24:405-415(2006); each of which is incorporated by reference herein.

Exemplary Cer and Sis sequences are provided in Xiang et al. J. Immunol. 190, 1819-1826 (2013) and Xiang et al. J. Immunol. 186, 5356-5366 (2011) which are incorporated by reference herein in their entireties. In some embodiments of any of the aspects, a Cer/Sis sequence can be a sequence having at least 80% sequence identity to to the ˜6.7 kb Cer/Sis sequence of SEQ ID NO: 13, e.g., 80% sequence identity, 85% sequence identity, 90% sequence identity, 95% sequence identity, 98% sequence identity, or greater sequence identity to SEQ ID NO:13. In some embodiments of any of the aspects, a Cer/Sis sequence can be a sequence having at least 95% identity to SEQ ID NO: 13 and the same activity e.g., CTCF-binding activity.

In some embodiments of any of the aspects, a Cer/Sis sequence can be a sequence having at least 80% sequence identity to bp 860-7288 of SEQ ID NO: 13, e.g., 80% sequence identity, 85% sequence identity, 90% sequence identity, 95% sequence identity, 98% sequence identity, or greater sequence identity to bp 860-7288 of SEQ ID NO: 13. In some embodiments of any of the aspects, a Cer/Sis sequence can be a sequence having at least 95% identity to bp 860-7288 of SEQ ID NO: 13 and the same activity e.g., CTCF-binding activity.

In some embodiments of any of the aspects, a Cer sequence can be a sequence having at least 80% sequence identity to bp 860-1529 of SEQ ID NO: 13, e.g., 80% sequence identity, 85% sequence identity, 90% sequence identity, 95% sequence identity, 98% sequence identity, or greater sequence identity to bp 860-1529 of SEQ ID NO:13. In some embodiments of any of the aspects, a Cer/Sis sequence can be a sequence having at least 95% identity to bp 860-1529 of SEQ ID NO: 13 and the same activity e.g., CTCF-binding activity.

In some embodiments of any of the aspects, a Sis sequence can be a sequence having at least 80% sequence identity to bp 3562-7288 of SEQ ID NO: 13, e.g., 80% sequence identity, 85% sequence identity, 90% sequence identity, 95% sequence identity, 98% sequence identity, or greater sequence identity to bp 3562-7288 of SEQ ID NO:13. In some embodiments of any of the aspects, a Cer/Sis sequence can be a sequence having at least 95% identity to bp 3562-7288 of SEQ ID NO: 13 and the same activity e.g., CTCF-binding activity.

Cer and Sis each comprise two CBEs. An exemplary murine wild-type sequence depicting Cer, Sis, and CBE elements is provided as Example 4 herein. Example 4 further demonstrates an exemplary embodiment of a deletion strategy using CRISPR/Cas9 technology to simultaneously delete both Cer and Sis elements (a total of ˜6.7 kb deletion). This deletion accordingly renders both the Cer and Sis non-functional, as detailed in Example 3. It is further contemplated herein that Cer and Sis block RAG scanning from the Jκ RC into the proximal Vκ domain.

Rendering the Cer/Sis sequence in the Igκ locus non-functional causes the 3′-most V_{L}segments to be subject to V(D)J recombination at an increased rate. In some embodiments of any of the aspects, the engineered IgL, or Igκ locus comprises a non-functional Cer/Sis sequence. A non-functional Cer/Sis sequence can be a Cer/Sis sequence which has 50% or less of the wild-type activity, e.g., 50% or less ability to attenuate VJ rearrangements with the 3′-most V_{L} segments. Methods of measuring the rate of VJ rearrangements comprising any given segment are known in the art, e.g., by HTGTS using Jκ bait primers (see, e.g. Lin et al. PNAS 113 (28) 7846-7851(2016); which is incorporated by reference herein in its entirety).

In some embodiments of any of the aspects, a non-functional Cer or Sis sequence is one in which at least one CBE sequence has been deleted. In some embodiments of any of the aspects, a non-functional Cer or Sis sequence is one in which both CBE sequences have been deleted. In some embodiments of any of the aspects, a non-functional Cer/Sis sequence is one in which all four CBE sequences have been deleted. In some embodiments of any of the aspects, a non-functional Cer/Sis sequence is one in which the Cer/Sis sequence has been deleted. In some embodiments of any of the aspects, a non-functional Cer/Sis sequence is one in which the Cer and/or Sis sequence has been deleted. In some embodiments of any of the aspects, a non-functional Cer/Sis sequence is one in which the Cer/Sis sequence has been deleted, e.g. the sequence corresponding to SEQ ID NO:13, bp 860-7288 of SEQ ID NO: 13, bp 860-1592 of SEQ ID NO: 13 and/or bp 3562-7288 of SEQ ID NO: 13 has been deleted.

In some embodiments of any of the aspects, a non-functional Cer/Sis sequence is one in which one or more CBE sequences have been deleted, e.g., a contiguous sequence comprising all four CBE sequences has been deleted, or any portion of the Cer/Sis comprising at least one CBE sequence has been deleted. In some embodiments of any of the aspects, a non-functional Cer/Sis sequence is one in which one or more CBE sequences have been mutated.

As used herein, “CTCF-binding element” or “CBE” refers to a nucleotide sequence bound by CTCF. A number of CBE's are known to exist in Ig loci, and further detail of CBE structure is provided, e.g., in Guo et al. Nature 2011 477-424-431; which is incorporated by reference herein in its entirety. In some embodiments of any of the aspects, a CBE can comprise or consist of any of SEQ ID Nos: 3-12.

(SEQ ID NO: 3)
GTATCAGCAGATGGCAGTG
(SEQ ID NO: 4)
GTGTCAGCAGATGGCAGAG
(SEQ ID NO: 5)
TGGCCACTTGAGGGAGCTA
(SEQ ID NO: 6)
TGGCCAGCAGAGGCCCCTA
CCGCGNGGNGGCAG
(SEQ ID NO: 7; CBE consensus sequence from Lee et
al. JBC 287:30906-30913 (2012))
CCACNAGGTGGCAG
(SEQ ID NO: 8; CBE consensus sequence from
Hu et al. Cell 163:947-959 (2015))
ATGGCCACAAGGGGGAAGC
(SEQ ID NO: 9; see, e.g., Guo et al., Nature 2011)
TCTCCACAAGAGGGCAGAA
(SEQ ID NO: 10; see, e.g., Guo et al., Nature
2011)
AGGACCAGCAGGGGGCGCGG
(SEQ ID NO: 11; see, e.g., Jain et al., Cell 2018)
GGACCAGCAGGGGGCAGTGA
(SEQ ID NO: 12; see, e.g., Jain et al., Cell 2018)

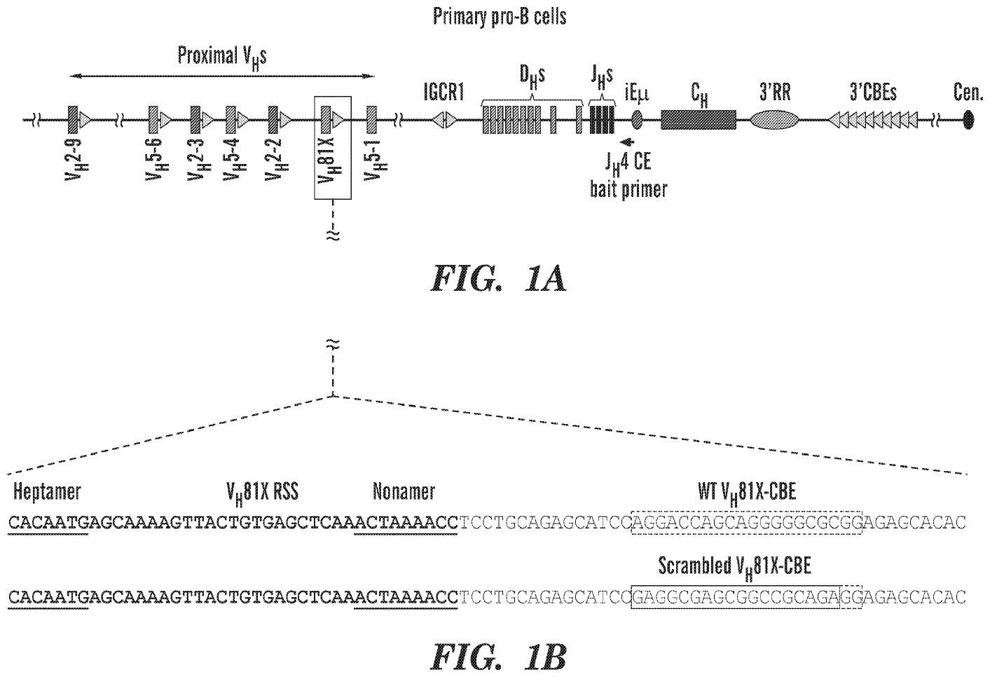

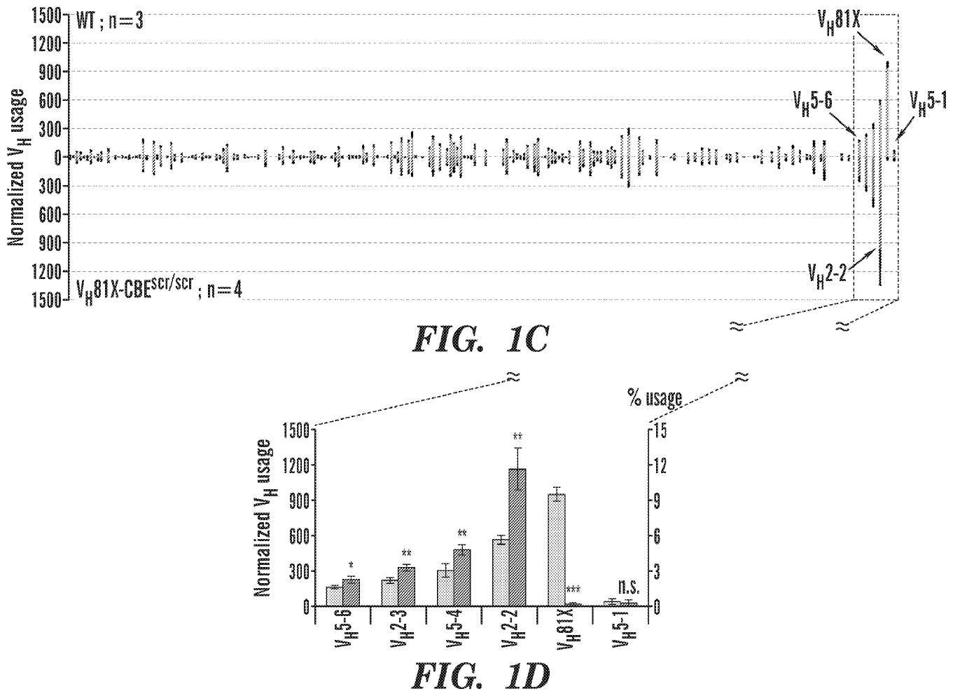

Further exemplary CBE sequences are described in Xiang et al. J. Immunol. 190, 1819-1826 (2013), which is incorporated by reference herein in its entirety, in which each of the two CBE sequences within both Cer and Sis elements (which are referred to therein as HS1-2 and HS3-6, respectively) are highlighted in FIG. 1C. In some embodiments of any of the aspects, a CBE can be a naturally-occurring murine or human CBE sequence.

CBEs can be rendered non-functional by, e.g., mutating the CBE or deleting the CBE. Mutating the sequence of a CBE sequence, such that CTCF binding is reduced by at least 25% (e.g. reduced by 25% or more, 50% or more, or 75% or more) can render the CBE non-functional. Binding of CTCF to a given mutated CBE can be readily measured, e.g., EMSA or ChIP)-Non-limiting examples of such mutations are described, e.g., in Guo et al. Nature 2011 477-424-431 and Jain et al., Cell (2018); which is incorporated by references herein in their entireties.

In some embodiments of any of the aspects, the CBE element is located 5′ of at least one V segment in the locus, e.g., the target V segment is not the 3′ most V segment. The CBE element is contemplated to be arranged in either orientation with respect to the target segment, e.g., it can be in the same orientation or inverted with respect to the target segment.

In some embodiments of any of the aspects, the CBE element can be contiguous with the target V segment. In some embodiments of any of the aspects, the CBE element can be 3′ of the target V segment's recombination signal sequence. In some embodiments of any of the aspects, the CBE element can be 1 bp or more 3′ of the target V segment's recombination signal sequence, e.g., 1 bp, 3 bp, 5 bp, 10 bp, 15 bp, or further 3′ of the target V segment's recombination signal sequence. In some embodiments of any of the aspects, the CBE element can be 15 bp or more 3′ of the target V segment's recombination signal sequence. In some embodiments of any of the aspects, the CBE element can be about 15 bp 3′ of the target V segment's recombination signal sequence.

In some embodiments of any of the aspects, the cell can further comprise an engineered non-functional IGCR1 sequence in the IgH within the nucleic acid sequence separating the 3′ end of the 3′-most V_{H} segment of the IgH locus and the 5′ end of a DH segment of the IgH locus. Rendering the IGCR1 sequence of an IgH locus non-functional causes the 3′-most V_{H} segment to be recombined into a VDJ segment at an even higher rate. In some embodiments, when the IGCR1 sequence is non-function, the VH segment which will recombine into a VDJ segment most frequently is the most 3′VH segment with an associated CBE just downstream of it (e.g., downstream of its RSS). Such a CBE can be naturally occurring engineered as described herein. In some embodiments of any of the aspects, the engineered IgH gene comprises a non-functional IGCR1 sequence. As used herein, “intergenic control region 1” or “IGCR1” refers to a region located in the IgH locus the 3′ end of the 3′-most native VH segment and the 5′ end of the 5′-most native DH segment and controls VDJ recombination. The IGCR1 is approximately 4.1 kb in length The IGCR1 comprises two CTCF-binding elements (CBEs) that are required for IGCR1 function. The structure of IGCR1 and the CBEs is explained in more detail, e.g., in Guo et al. Nature 2011 477-424-431; which is incorporated by reference herein in its entirety. A non-functional IGCR1 sequence can be an IGCR1 sequence which has 50% or less of the wild-type activity, e.g., 50% or less ability to form V(D)J rearrangements with V_{H} segments other than the 3′-most V_{H}segment. Methods of measuring the rate of VDJ rearrangements comprising any given segment are known in the art, e.g., by HTGTS using JH bait primers (see, e.g., Lin et al. PNAS 113 (28) 7846-7851(2016); which is incorporated by reference herein in its entirety.)

In some embodiments of any of the aspects, a non-functional IGCR1 sequence is one in which at least one CBE sequence has been deleted. In some embodiments of any of the aspects, a non-functional IGCR1 sequence is one in which both CBE sequences have been deleted. In some embodiments of any of the aspects, a non-functional IGCR1 sequence is one in which the IGCR1 sequence has been deleted, e.g. the 4.1 kb comprising IGCR1 has been deleted. In some embodiments of any of the aspects, a non-functional IGCR1 sequence is one in which one or more CBE sequences have been deleted, e.g., the 2.6 kb sequence comprising both CBE sequences has been deleted, or any portion of that 2.6 kb sequence comprising at least one CBE sequence has been deleted. In some embodiments of any of the aspects, a non-functional IGCR1 sequence is one in which one or more CBE sequences have been mutated. Mutating the sequence of a CBE sequence, such that CTCF binding is reduced by at least 25% (e.g. reduced by 25% or more, 50% or more, or 75% or more) can render the IGCR1 non-functional. Binding of CTCF to a given mutated CBE can be readily measured, e.g., EMSA or ChIP. Non-limiting examples of such mutations are described, e.g., in Guo et al. Nature 2011 477-424-431; and Jain et al., Cell (2018) which is incorporated by reference herein in its entirety.

If a particular V_{H} segment, J_{H} segment, D segment, assembled DJH segment, assembled V_{H}DJ_{H} segment, heavy chain sequence, V_{L} segment, J_{L} segment, assembled V_{L}J_{L} segment, and/or light chain sequence is desired to be present in the mature antibody or antibodies produced by a cell and/or animal described herein, the IgH and/or IgL locus can be further engineered to comprise such a sequence of interest. In some embodiments of any of the aspects, the locus can be engineered to comprise the sequence of interest such that it is one possible segment of its type that can be recombined to form a mature antibody sequence (e.g. a human J_{H} segment can be introduced into a murine IgH locus while retaining at least one native mouse J_{H} segment). In some embodiments of any of the aspects, the locus can be engineered to comprise the sequence of interest such that it will be the segment of its type that will be present in all mature antibody sequences (e.g., a human J_{H} segment or human DJH intermediate can be introduced into a murine IgH locus in which all native murine J_{H} segments are deleted or disabled).

In some embodiments of any of the aspects, the J_{H} locus can be replaced by a human D and J_{H}cassette or a cassette with an assembled human DJH. In some embodiments of any of the aspects, one or more D_{H}, one or more J_{H} segments, and/or a DJH fusion comprise a cassette targeting sequence. In some embodiments of any of the aspects, the IgH locus comprises one or more non-native D_{H} segments. In some embodiments of any of the aspects, the IgH locus comprises one D_{H} segment. In some embodiments of any of the aspects, the IgH locus comprises one or more non-native J_{H} segments. In some embodiments of any of the aspects, the IgH locus comprises one J_{H} segment. In some embodiments of any of the aspects, the IgH locus comprises murine IgH locus sequence. In some embodiments of any of the aspects, the IgH locus comprises human IgH locus sequence. In some embodiments of any of the aspects, the locus comprises humanized IgH locus sequence.

In some embodiments of any of the aspects, the IgL locus comprises one or more non-native J_{L} segments. In some embodiments of any of the aspects, the IgL locus comprises one J_{L} segment. In some embodiments of any of the aspects, the IgL locus comprises murine IgL locus sequence. In some embodiments of any of the aspects, the IgL locus comprises human IgL locus sequence. In some embodiments of any of the aspects, the locus comprises humanized IgL locus sequence.

In some embodiments of any of the aspects, the IgL locus can be engineered to comprise human sequence, to be a humanized IgL locus, or to be a human IgL locus. In some embodiments of any of the aspects, the IgH locus can be engineered to comprise human sequence, to be a humanized IgH locus, or to be a human IgH locus.

In some embodiments of any of the aspects, a cell described herein can comprise an IgL locus with one V_{L} segment. In some embodiments of any of the aspects, a cell described herein can comprise an IgL locus with one J_{L} segment. In some embodiments of any of the aspects, a cell described herein can comprise a human rearranged V_{L}J_{L} at the IgL locus. In some embodiments of any of the aspects, the IgL gene encodes IGκV1.

In some embodiments of any of the aspects, a cell described herein can comprise an IgH locus with one V_{H} segment. In some embodiments of any of the aspects, a cell described herein can comprise an IgH locus with one D segment. In some embodiments of any of the aspects, a cell described herein can comprise an IgH locus with one J_{H} segment. In some embodiments of any of the aspects, a cell described herein can comprise a human rearranged V_{H}DJ_{H} at the IgH locus.

The methods and compositions described herein can relate to the production of antibodies in a manner that capitalizes on the variation produced by, e.g., the GC response and SHM. In some embodiments of any of the aspects, a cell described herein can further comprise a mutation capable of activating, inactivating or modifying genes that in a lymphocyte-intrinsic fashion lead to increased GC antibody maturation responses. Such mutations are known in the art and can include, by way of non-limiting example PTEN^{-/-} (see, e.g., Rolf et al. Journal of Immunology 2010 185:4042-4052; which is incorporated by reference herein in its entirety)/

In some embodiments of any of the aspects, the Ig locus and/or target segment can further comprise a cassette targeting sequence, e.g., to permit insertion and/or replacement of sequences in the Ig locus and/or target segment. As used herein, the term “cassette targeting sequence” refers to a sequence that permits a sequence of interest (e.g. a sequence comprising a V segment of interest), to be inserted into the genome at the location of the cassette targeting sequence via the action of at least one enzyme that targets the cassette targeting sequence. Non-limiting examples of cassette targeting sequences are an I-SceI meganuclease site; a Cas9/CRISPR target sequence; a Talen target sequence; a zinc finger nuclease (ZFN) and a recombinase-mediated cassette exchange system. Such cassette targeting systems are known in the art, see, e.g. Clark and Whitelaw Nature Reviews Genetics 2003 4:825-833; which is incorporated by reference herein in its entirety. In some embodiments of any of the aspects, the cassette targeting sequence permits the replacement of the 3′-most V_{H} segment.

I-SceI, Zinc finger nucleases (ZFNs), the Cas9/CRISPR system, and transcription-activator like effector nucleases (TALENs) are nucleases. Nucleases are found commonly in microbial species and have the unique property of having very long recognition sequences (>14 bp) thus making them naturally very specific for cutting at a desired location. This can be exploited to make site-specific double-stranded breaks in, e.g. a genome. These nucleases can cut and create specific double-stranded breaks at a desired location(s) in the genome, which are then repaired by cellular endogenous processes such as, homologous recombination (HR), homology directed repair (HDR) and non-homologous end-joining (NHEJ). NHEJ directly joins the DNA ends in a double-stranded break, while HDR utilizes a homologous sequence as a template for regenerating the missing DNA sequence at the break point. Thus, by introducing, e.g., a ZFN, CRISPR, and/or TALENs specific for the cassette targeting sequence into a cell, at least one double strand-break can be generated in the genome, resulting in a template sequence, e.g. a sequence comprising a segment of interest, being used to repair the break, thereby introducing the template sequence into the genome and the desired location (see, e.g. Gaj et al. Trends in Biotechnology 2013 31:397-405; Carlson et al. PNAS 2012 109:17382-7; and Wang et al. Cell 2013 153:910-8; each of which is incorporated by reference herein in its entirety).

Mutagenesis and high throughput screening methods have been used to create nuclease and/or meganuclease variants that recognize unique sequences. For example, various nucleases have been fused to create hybrid enzymes that recognize a new sequence. Alternatively, DNA interacting amino acids of the nuclease can be altered to design sequence specific nucleases (see e.g., U.S. Pat. No. 8,021,867). Nucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8,163,514, the contents of each are incorporated herein by reference in their entirety. Alternatively, nucleases with site specific cutting characteristics can be obtained using commercially available technologies e.g., Precision BioSciences' Directed Nuclease Editor™ genome editing technology.

ZFNs and TALENs restriction endonuclease technology utilizes a non-specific DNA cutting enzyme which is linked to a specific DNA sequence recognizing peptide(s) such as zinc fingers and transcription activator-like effectors (TALEs). Typically, an endonuclease whose DNA recognition site and cleaving site are separate from each other is selected and its cleaving portion is separated and then linked to a sequence recognizing peptide, thereby yielding an endonuclease with very high specificity for a desired sequence. An exemplary restriction enzyme with such properties is FokI. Additionally, FokI has the advantage of requiring dimerization to have nuclease activity and this means the specificity increases dramatically as each nuclease partner recognizes a unique DNA sequence. To enhance this effect, FokI nucleases have been engineered that can only function as heterodimers and have increased catalytic activity. The heterodimer functioning nucleases avoid the possibility of unwanted homodimer activity and thus increase specificity of the double-stranded break.

In some embodiments of any of the aspects, the Cas9/CRISPR system can be used to introduce sequences at a cassette targeting sequence as described herein. Clustered regularly interspaced short palindromic repeats (CRISPR)/CRISPR-associated (Cas) systems are useful for, e.g. RNA-programmable genome editing (see e.g., Marraffini and Sontheimer. Nature Reviews Genetics 2010 11:181-190; Sorek et al. Nature Reviews Microbiology 2008 6:181-6; Karginov and Hannon. Mol Cell 2010 1:7-19; Hale et al. Mol Cell 2010:45:292-302; Jinek et al. Science 2012 337:815-820; Bikard and Marraffini Curr Opin Immunol 2012 24:15-20; Bikard et al. Cell Host & Microbe 2012 12:177-186; all of which are incorporated by reference herein in their entireties). A CRISPR guide RNA is used that can target a Cas enzyme to the desired location in the genome, where it generates a double strand break. This technique is known in the art and described, e.g. at Mali et al. Science 2013 339:823-6; which is incorporated by reference herein in its entirety and kits for the design and use of CRISPR-mediated genome editing are commercially available, e.g. the PRECISION X CAS9 SMART NUCLEASE™ System (Cat No. CAS900A-1) from System Biosciences, Mountain View, CA.

In some embodiments of any of the aspects, a CRISPR, TALENs, or ZFN molecule (e.g. a peptide and/or peptide/nucleic acid complex) can be introduced into a cell, e.g. a cultured ES cell, such that the presence of the CRISPR, TALENs, or ZFN molecule is transient and will not be detectable in the progeny of, or an animal derived from, that cell. In some embodiments of any of the aspects, a nucleic acid encoding a CRISPR, TALENs, or ZFN molecule (e.g. a peptide and/or multiple nucleic acids encoding the parts of a peptide/nucleic acid complex) can be introduced into a cell, e.g. a cultured ES cell, such that the nucleic acid is present in the cell transiently and the nucleic acid encoding the CRISPR, TALENs, or ZFN molecule as well as the CRISPR, TALENs, or ZFN molecule itself will not be detectable in the progeny of, or an animal derived from, that cell. In some embodiments of any of the aspects, a nucleic acid encoding a CRISPR, TALENs, or ZFN molecule (e.g. a peptide and/or multiple nucleic acids encoding the parts of a peptide/nucleic acid complex) can be introduced into a cell, e.g. a cultured ES cell, such that the nucleic acid is maintained in the cell (e.g. incorporated into the genome) and the nucleic acid encoding the CRISPR, TALENs, or ZFN molecule and/or the CRISPR, TALENs, or ZFN molecule will be detectable in the progeny of, or an animal derived from, that cell.

Recombinase-mediated cassette exchange systems (RMCEs) utilize recombinases (e.g. Flp) and the sequences recognized by the recombinases (e.g., FRT target sites) to swap sequences from the genome, flagged by the FRT target sites with sequences in a cassette that are likewise flanked by the FRT target sites. RMCEs are known in the art, e.g., Cesari et al. Genesis 2004 38:87-92 and Roebroek et al. Mol Cell Biol 2006 26:605-616; each of which is incorporated by reference herein in its entirety.

It can be difficult to isolate and/or produce antibodies comprising a particular segment (e.g., V segment) because that segment is selected against, e.g. if that segment is particularly likely to recognize a self-antigen, B-cells with the segment are more likely to be selected against. Such segments are termed “maturation-incompatible” herein. This term does not imply that B-cells expressing a BCR and/or antibody comprising such a segment are invariably subject to clonal deletion and/or anergy. Provided herein are methods and compositions for avoiding clonal deletion and/or anergy during B-cell development and causing B-cells to express a maturation-incompatible segment at a desired timepoint in development, e.g. after clonal deletion and/or anergy is likely to occur. These methods and compositions involve inserting a passenger V(D)J exon into a Ig locus in such a manner that while present in the locus, it will be neither expressed nor removed by normal Ig V(D)J recombination. A B cell comprising the passenger V(D)J exon will express a second, maturation-compatible, V(D)J exon (e.g. one generated by Ig V(D)J recombination) and at a desired time, the sequence of the locus can be manipulated to cause the passenger V(D)J exon to be expressed instead of the maturation-compatible exon. As used herein, a “passenger” exon is an exon that is present in the germline and mature B-cell genome but is not expressed until the genome is subjected to an induced recombination event, e.g. a Cre-mediated recombination event.

In a first approach, the maturation-incompatible segment (e.g. as part of a passenger V(D)J exon) is inserted into the Ig locus in a 3′ to 5′ conformation relative to the Ig locus and is located 5′ of the maturation-compatible V(D)J exon (or the sequences that will be recombined to make the maturation-compatible V(D)J exon). Expression of the passenger V(D)J exon is induced by the use of a pair of inverted recombinase sites, which cause the passenger V(D)J exon to be “flipped” so that it is in the 5′ to 3′ orientation with respect to the rest of the Ig locus. In a second approach, the maturation-incompatible segment, (e.g. as part of a passenger V(D)J exon) is inserted 5′ to 3′ with respect to the Ig locus and V(D)J recombination occurs downstream of the passenger exon to generate a maturation-compatible V(D)J exon. The maturation-compatible V(D)J exon can then be excised by inducing recombination (e.g., Cre-mediated recombination) at a pair of recombinase sites when desired, causing the cell to express the passenger exon. As an illustrative example, a known functional driver V(D)J exon can be used to permit B cell development with a passenger exon just upstream and not expressed due to transcriptional terminators or other blocks. The driver and transcription blocks are flanked by loxP elements and deleted by CD21 cre expression in periphery to allow passenger expression. This approach has been used successfully to express several HIV bnAB V(D)J intermediates that otherwise could not be expressed in periphery.

Recombination sites and systems for inducing recombination at these sites are known in the art, e.g. the cre-Lox system or the Flp recombinase. The loxP-Cre system utilizes the expression of the PI phage Cre recombinase to catalyze the excision or inversion of DNA located between flanking lox sites. By using gene-targeting techniques to produce binary transgene animals with modified endogenous genes that can be acted on by Cre or Flp recombinases expressed under the control of tissue-specific promoters, site-specific recombination may be employed to excise or invert sequences in a spatially or time-controlled manner. See, e.g., U.S. Pat. Nos. 6,080,576, 5,434,066, and 4,959,317; and Joyner, A. L., et al. Laboratory Protocols for Conditional Gene Targeting, Oxford University Press, New York (1997); Orban et al. (1992) PNAS 89:6861-6865; Aguzzi A, Brandner S, Isenmann S, Steinbach JP, Sure U. Glia. 1995 November; 15(3):348-64. Review; each of which is incorporated by reference herein in its entirety.

In some embodiments of any of the aspects, the cell further comprises a gene encoding a recombinase that will induce recombination at the recombinase site. In some embodiments of any of the aspects, the recombinase site is a LoxP site. In some embodiments of any of the aspects, the cell further comprises a gene encoding cre recombinase. A gene encoding a recombinase can be under the control of, e.g. an inducible promoter or a cell-specific promoter. Inducible promoters, temporally-specific, and tissue-specific promoters for the control of a recombinase are known in the art. In some embodiments of any of the aspects, the gene encoding a recombinase is under the control of a promoter which is not active in immature B cells and is active in peripheral B cells, e.g. the CD21 promoter, CD84 promoter. In some embodiments of any of the aspects, the gene encoding the recombinase is not active in all mature B cells but is preferentially expressed in germinal center B cells. Exemplary promoters for germinal center specific, or at least biased, expression include, but are not limited to, the Iγ1 or AID promoters.

In some embodiments of any of the aspects, the cell is heterozygous for the engineered Ig locus (or loci) as described herein and the other Ig locus or (loci) has been engineered to be inactive, wherein the cell will express an Ig chain only from the engineered Ig locus as described herein. The inactive Ig locus can be, by way of non-limiting example, deleted, partially deleted, and/or mutated (e.g. to inactivate sequences necessary for V(D)J recombination can be mutated and/or deleted (e.g. deleting the J_{H} portion of the locus).

To further address whether Human VκJκ repertoires might show increased junctional diversity versus those of mouse VkJk repertoires, HTGTS-Rep-seq analysis was performed on DNA from WT mouse IgM+ splenic B cells and human peripheral blood mononuclear cells (PBMCs) using a mouse or human JK1 bait as a primer. To obviate the possibility of influences of cellular selection, presented are results of out of frame (non-productive) VκJκ junctions. This analysis demonstrated a markedly greater incorporation of P and/or N junctional elements into the human VκJκ junctions versus the mouse VκJκ junctions (FIG. 20). These findings support the incorporation of enforced TdT expression into the engineered cells and/or mammals described herein to permit generation of a more human-like Igκ repertoire. Additionally, it is contemplated herein that IgL repertoire diversity can be increased, particularly in murine cells, by increasing the expression of TdT. TdT (Terminal deoxynucleotidyl transferase), or DNA nucleotidylexotransferase, is a polypepide that introduces non-templated nucleotides into V, D, and J exons during V(D)J recombination to greatly diversify antibody repertoires (Alt and Baltimore, 1982). Accordingly, in some embodiments of any of the aspects described herein, the cells can further comprise an exogenous and/or non-native nucleic acid sequence encoding TdT. Nucleic acid sequence encoding for TdT for a number of species are known in the art, e.g., human TdT (NCBI Gene ID: 1791; e.g., NM_001017520.1 and NM_004088.3) and murine TdT (NCBI Gene ID: 21673; e.g., NM_001043228.1 and NM_009345.2). The TdT can be human TdT or murine TdT. The TdT can be one of the foregoing reference sequences or a variant, homolog, ortholog, or allele thereof.

In some embodiments of any of the aspects, the TdT sequence can be operably linked to a promoter, e.g., a promoter active in B cells. In some embodiments of any of the aspects, the promoter is a strong promoter, a constitutively active promoter, and/or a synthertic promoter. Exemplary but non-hinting promoters are the “CAG” promoter—a combined sequences of cytomegalovirus (CMV) early enhancer element (“C”), the promoter, the first exon and the first intron of chicken beta-actin gene (“A”), and the splice acceptor of the rabbit beta-globin gene (“G”)), the Eμ-N-myc promoter (Bentolila et al., J I 158(2):715-723 (1997)), or other promoters that enforce TDT expression in developing pro and pre B lymphocytes. In some embodiments of any of the aspects, the TdT-encoding sequence can be present in a vector and/or stably integrated into the genome of the cell (e.g., at the Rosa26 locus) that is stably integrated into the constitutively expressed mouse Rosa26 locus.

In some embodiments of any of the aspects, a nucleic acid encoding a polypeptide as described herein (e.g. a TdT polypeptide) is comprised by a vector. In some of the aspects described herein, a nucleic acid sequence encoding a given polypeptide as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art.

By “recombinant vector” is meant a vector that includes a heterologous nucleic acid sequence, or “transgene” that is capable of expression in vivo. It should be understood that the vectors described herein can, In some embodiments of any of the aspects, be combined with other suitable compositions and therapies. In some embodiments of any of the aspects, the vector is episomal. The use of a suitable episomal vector provides a means of maintaining the nucleotide of interest in the subject in high copy number extra chromosomal DNA thereby eliminating potential effects of chromosomal integration.

In some embodiments of any of the aspects, described herein is a cell comprising: a) an engineered IgH locus comprising at least one of:
i. a CBE element within the nucleic acid sequence separating the 3′ end of a target V_{H} segment and the 5′ end of the first V_{H} segment which is 3′ of the target V_{H}segment;
ii. an engineered non-functional IGCR1 sequence in the IgH locus within the nucleic acid sequence separating the 3′ end of the 3′-most V_{H} segment of the IgH locus and the 5′ end of a D_{H} segment of the IgH locus; and/or
   b) an engineered IgL locus comprising at least one of:
iii. a non-functional Cer/Sis sequence within the nucleic acid sequence separating the 3′ end of the 3′-most V_{L} segment and the 5′ end of a J_{L} segment; and
iv. a CBE element within the nucleic acid sequence separating the 3′ end of a target V_{L} segment and the 5′ end of the first V_{L} segment which is 3′ of the target V_{L}segment.
   In some embodiments of any of the aspects, described herein is a mammal comprising at least one cell, or a population of cells comprising: a) an engineered IgH locus comprising at least one of:
i. a CBE element within the nucleic acid sequence separating the 3′ end of a target V_{H} segment and the 5′ end of the first V_{H} segment which is 3′ of the target V_{H}segment;
ii. an engineered non-functional IGCR1 sequence in the IgH locus within the nucleic acid sequence separating the 3′ end of the 3′-most V_{H} segment of the IgH locus and the 5′ end of a D_{H} segment of the IgH locus; and/or
   b) an engineered IgL locus comprising at least one of:
iii. a non-functional Cer/Sis sequence within the nucleic acid sequence separating the 3′ end of the 3′-most V_{L} segment and the 5′ end of a J_{L} segment; and
iv. a CBE element within the nucleic acid sequence separating the 3′ end of a target V_{L} segment and the 5′ end of the first V_{L} segment which is 3′ of the target V_{L}segment;
   whereby V(D)J recombination in the mammal predominantly utilizes the target V_{H} segment and the target V_{L} segment. In some embodiments of any of the aspects, the IgH locus is further engineered to comprise one target D segment and/or one target J_{H} segment, one DJ_{H} rearrangement, and/or the IgL locus is further engineered to comprise one target J_{L} segment. In some embodiments of any of the aspects, the engineered IgH locus is further engineered to comprise only one V_{H} segment (e.g., one human V_{H} segment), and/or the engineered IgL locus is further engineered to comprise only one V_{L} segment (e.g., one human V_{L}segment). In some embodiments of any of the aspects, the target segments are human segments. Particularly when the cells are engineered such that the target segments are those utilized in a known antibody, such cells and/or mammals permit development of large, diverse B cell repetoires which comprise variants of the known antibody with improved specificity and/or affinity.

A cell as described herein can be, by way of non-limiting example, a stem cell, an embryonic stem cell, a B cell, a mature B cell, an immature B cell, and/or a hybridoma cell. A cell as described herein can be, by way of non-limiting example, a mammalian cell, a human cell, and/or a mouse cell. In some embodiments of any of the aspects, a cell as described herein can be a mouse embryonic stem cell.

In one aspect, described herein is genetically engineered mammal comprising an engineered cell as described herein. In some embodiments of any of the aspects, the mammal can be a mouse. In some embodiments of any of the aspects, the methods described herein, e.g. methods of producing antibodies and/or testing antigens require only that the B-cells of the genetically engineered mammal are engineered as described herein. Accordingly, in some embodiments of any of the aspects, the genetically engineered mammal can be a chimera, e.g. it can comprise two genetically distinct populations of cells. The use of chimeras can expedite the process of obtaining a genetically engineered mammal to be used in the methods described herein. In one aspect, described herein is a chimeric genetically engineered mammal, e.g. a mouse, comprising two populations of cells, a first population comprising cells which are V(D)J recombination-defective; and a second population comprising engineered cells as described herein. V(D)J recombination-defective cells mice are known in the art, e.g. RAG2^{-/-} cells.

In some embodiments of any of the aspects, the mammal, e.g., the genetically engineered mammal described herein, is a mouse.

In one aspect of any of the embodiments, provided herein is a set of at least two mammals, wherein each mammal is a mammal comprising an engineered Ig locus(loci) as described herein, the first mammal comprising a first target V_{H} segment and/or a first target V_{L} segment and each further mammal comprising a further target V_{H} segment and/or a further target V_{L} segment. In some embodiments of any of the aspects, each mammal comprises a human target V_{H} segment and a human target V_{L} segment.

For example, a mammal with an engineered IgH locus can be bred with a mammal with an engineered IgL locus to make a system in which the derived mammal would have both IgH and Igk rearranging loci. Such animals can be used for immunization to discover and or optimize novel humanized antibodies. Sets of such mammals can be provided for each of the frequently utilized human VHs and VL so that multiple combinations are available within the set. In some embodiments, the mice can have IGCR1 deleted for IgH with human VH replacing VH 81X (with its own CBE) or more proximal VH5-1 (with added CBE) and Igκ locus with Cer/Sis deleted and proximal Vκ replaced with human Vκ or Vλ (e.g., with replace Vλ23 RSS replaced with Vκ 12RSS to preserve pairing with Jκ 23RSS). Such mammals can also be produced by engineering all mutations in a single ES cell and reconstituting B cells (and T cells) in RAG-deficient chimeras for immunization via a RAG blastocyste complementation approach (e.g. see Tian et al., 201i6 which is incorporated by reference herein in its entirety).

The cells and mammals described herein permit the optimization, improvement, or modification of known antibodies. By engineering the cell and/or mammal to express antibodies (which are subject to V(D)J recombination, the GC reaction, and/or SHM), comprising segment(s) known to recognize a particular antigen (e.g. segment(s) from a known antibody that recognizes the particular antigen), a large number of precursor antibodies can be generated which are related to and/or derived from segments of the known antibody. These antibodies can be screened and/or selected, in vitro and/or in vivo for optimized characteristics relative to the known antibody. Optimization can be an increase in, e.g. affinity, breadth, and/or specificity or other desired characteristics.

In one aspect, described herein is method of making an optimized antibody from a known antibody, the method comprising the steps of: injecting a mouse blastocyst with a cell as described herein, wherein the cell is a mouse embryonic stem cell, and wherein the target segment comprises the V_{H} or V_{L}segment of a known antibody; implanting the mouse blastocyst into a female mouse under conditions suitable to allow maturation of the blastocyst into a genetically engineered mouse; and isolating 1) an optimized antibody comprising the non-native V segment; or 2) a cell producing an optimized antibody comprising the non-native V segment from the genetically engineered mouse. In some embodiments of any of the aspects, the blastocyst cells are V(D)J recombination-defective cells, e.g. RAG2^{-/-} cells. In some embodiments of any of the aspects, the IgH and/or IgL loci of the blastocyst cells have been rendered non-functional, as described elsewhere herein. In some embodiments of any of the aspects, the blastocyst cells are not capable of forming mature B cells, and optionally are not capable of forming mature T-cells. In some embodiments of any of the aspects, the blastocyst cells are not capable of forming mature lymphocytes.

In some embodiments of any of the aspects, the method can further comprise a step of immunizing the genetically engineered mouse with a desired target antigen before the isolating step. In some embodiments of any of the aspects, the method can further comprise a step of producing a monoclonal antibody from at least one cell of the genetically engineered mouse.

Once the cell as described herein is produced through the methods described herein, an animal can be produced from this cell through either stem cell technology or cloning technology. For example, if the cell into which the nucleic acid was transfected was a stem cell for the organism (e.g. an embryonic stem cell), then this cell, after transfection and culturing, can be used to produce an organism which will contain the engineered aspects in germline cells, which can then in turn be used to produce another animal that possesses the engineered aspects in all of its cells. In other methods for production of an animal containing the engineered aspects, cloning technologies can be used. These technologies generally take the nucleus of the engineered cell and either through fusion or replacement fuse the engineered nucleus with an oocyte which can then be manipulated to produce an animal. The advantage of procedures that use cloning instead of ES technology is that cells other than ES cells can be transfected. For example, a fibroblast cell, which is very easy to culture can be used as the cell which is engineered, and then cells derived from this cell can be used to clone a whole animal.

Production of the engineered animals described herein can, in some embodiments, also utilize RAG2-deficient blastocyst complementation (RDBC) technology, which is known in the art and described, e.g., in Chen et al. PNAS 90:4528-4532 (1993); Tian et al., Cell 166:1471-1484(2016); which are incorporated by reference herein in their entireties.

The engineered animals described herein can also be produced by zygote micro-injection/electroporation. Such methods are known in the art and described at, e.g., Wang et al. Cell. 2013; 153(4):910-8; Yang et al. Cell. 2013; 154(6):1370-9; Yasue et al. Scientific reports. 2014; 4:5705; Hashimoto et al. Developmental biology. 2016; 418(1):1-9; and Wang et al. BioTechniques. 2015; 59(4):201-2, 4, 6-8; each of which is incorporated by reference herein in its entirety.

Generally, cells (e.g. ES cells) used to produce the engineered animals will be of the same species as the animal to be generated. Thus, for example, mouse embryonic stem cells will usually be used for generation of engineered mice. Methods of isolating, culturing, and manipulating various cells types are known in the art. By way of non-limiting example, embryonic stem cells are generated and maintained using methods well known to the skilled artisan such as those described by Doetschman et al. (1985) J. Embryol. Exp. Mol. Biol. 87:27-45). The cells are cultured and prepared for genetic engineering using methods well known to the skilled artisan, such as those set forth by Robertson in: Teratocarcinomas and Embryonic Stem Cells: A Practical Approach, E. J. Robertson, ed. IRI. Press, Washington, D.C. [1987]); by Bradley et al. (1986) Current Topics in Devel. Biol. 20:357-371); and by Hogan et al. (Manipulating the Mouse Embryo: A Laboratory Manual, Cold Spring Harbor Laboratory Press, Cold Spring Harbor, N.Y. (1986)).

In some embodiments of any of the aspects, after cells comprising the engineered aspects have been generated, and optionally, selected, the cells can be inserted into an embryo or blastocyst, e.g. to generate a chimera. Insertion may be accomplished in a variety of ways known to the skilled artisan, however the typical method is by microinjection. For microinjection, about 10-30 cells are collected into a micropipet and injected into embryos that are at the proper stage of development to permit integration of the engineered ES cell into the developing embryo or blastocyst. For instance, the ES cells can be microinjected into blastocysts. The suitable stage of development for the embryo used for insertion of ES cells is very species dependent, however for mice it is about 3.5 days. The embryos are obtained by perfusing the uterus of pregnant females. Suitable methods for accomplishing this are known to the skilled artisan.

Methods of isolating antibodies and/or antibody-producing cells are known in the art, and can include, by way of non-limiting example, producing a monoclonal antibody via, e.g., the production of hybridomas or phage display. See, e.g., Little et al. Immunology Today 2000 21:364-370; Pasqualini et al. PNAS 2004 101:257-259; Reichert et al. Nature Reviews Drug Discovery 2007 6:349-356; and Wang et al. Antibody Technology Journal 2011 1:1-4; each of which is incorporated by reference herein in its entirety.

In one aspect, described herein is an optimized antibody produced by the method described above herein.

Certain vaccine development strategies rely upon identifying one or more intermediate antigens, such that immunization with the one or more intermediate antigens will trigger B cell activation and diversification of antibodies, resulting in the production of an antibody that will recognize the final target antigen (e.g. an HIV antigen). Accordingly, described herein are methods and compositions that permit the in vivo evaluation of such intermediate antigens. In some embodiments of any of the aspects, structural information about antibodies that will recognize the final target antigen is known, e.g. what V_{H} or V_{L} segment is comprised by antibodies to HIV antigens in those rare subjects with a natural antibody defense against HIV. Using the methods and compositions described herein, the ability of an intermediate antigen to activate B cells comprising antibodies with such a V_{H} or V_{L} segment can be assessed, permitting the development of multiple antigen immunization therapies.

In one aspect, described herein is a method of identifying a candidate antigen as an antigen that activates a B cell population comprising a V segment of interest, the method comprising: immunizing an engineered mammal as described herein, engineered such that a majority of the mammal's peripheral B cells express the V segment(s) of interest, with the antigen; measuring B cell activation in the mammal; and identifying the candidate antigen as an activator of a B cell population comprising the V segment(s) of interest if the B cell activation in the mammal is increased relative to a reference level. B cell activation can be, e.g. an increase in the somatic hypermutation status of the Ig variable region, an increase in the affinity of mature antibodies for the antigen, and/or an increase in the specificity of mature antibodies for the antigen. As used herein, the term “activator,” as used in reference to activation of B cells refers to an antigen that increases B cell activation, e.g. increases B cell proliferation, SHM, and/or the GC reaction.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments of any of the aspects, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments of any of the aspects, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “highly-utilized” segment is a segment which is found, on average, in at least 3% of a naturally-generated antibody repertoire of a wild-type animal. In some embodiments of any of the aspects, the antibody repertoire can be an unselected repertoire. Highly-utilized segments are known in the art for a human of species. For example, non-limiting examples of highly-utilized segments can include IGHV1-2*02, IGHV1-69, V_{H}3-30, V_{H}4-59, Vκ1-5, Vκ3-20, Vκ4-1, Vλ1-51, Vλ3-1, and Vλ2-14.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments of any of the aspects, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to the assays described below herein. A functional fragment can comprise conservative substitutions of the sequences disclosed herein.

In some embodiments of any of the aspects, the polypeptide described herein can be a variant of a sequence described herein. In some embodiments of any of the aspects, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

As used herein, the term “nucleic acid” or “nucleic acid sequence” refers to any molecule, preferably a polymeric molecule, incorporating units of ribonucleic acid, deoxyribonucleic acid or an analog thereof The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

As used herein an “antibody” refers to IgG, IgM, IgA, IgD or IgE molecules or antigen-specific antibody fragments thereof (including, but not limited to, a Fab, F(ab′)₂, Fv, disulphide linked Fv, scFv, single domain antibody, closed conformation multispecific antibody, disulphide-linked scfv, diabody), whether derived from any species that naturally produces an antibody, or created by recombinant DNA technology; whether isolated from serum, B-cells, hybridomas, transfectomas, yeast or bacteria.

In another example, an antibody includes two heavy (H) chain variable regions and two light (L) chain variable regions. It should be noted that a VH region (e.g. a portion of an immunoglobin polypeptide is not the same as a VH segment, which is described elsewhere herein). The V_{H} and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917; which are incorporated by reference herein in their entireties). Each VH and VL is typically composed of three CDRs and four FRs, arranged from amino-terminus to carboxy-terminus in the following order: FRI, CDR1, FR2, CDR2, FR3, CDR3, FR4.

The term “monospecific antibody” refers to an antibody that displays a single binding specificity and affinity for a particular target, e.g., epitope. This term includes a “monoclonal antibody” or “monoclonal antibody composition,” which as used herein refer to a preparation of antibodies or fragments thereof of single molecular composition, irrespective of how the antibody was generated.

As described herein, an “antigen” is a molecule that is bound by a binding site on an antibody. Typically, antigens are bound by antibody ligands and are capable of raising an antibody response in vivo. An antigen can be a polypeptide, protein, nucleic acid or other molecule or portion thereof The term “antigenic determinant” refers to an epitope on the antigen recognized by an antigen-binding molecule, and more particularly, by the antigen-binding site of said molecule.

As used herein, the term “affinity” refers to the strength of an interaction, e.g. the binding of an antibody for an antigen and can be expressed quantitatively as a dissociation constant (κ_{D}). Avidity is the measure of the strength of binding between an antigen-binding molecule (such as an antibody reagent described herein) and the pertinent antigen. Avidity is related to both the affinity between an antigenic determinant and its antigen binding site on the antigen-binding molecule, and the number of pertinent binding sites present on the antigen-binding molecule. Typically, antigen-binding proteins (such as an antibody reagent described herein) will bind to their cognate or specific antigen with a dissociation constant (K_{D} of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁻⁵ to 10⁻¹² liter/moles or more, and preferably 10⁷ to 10¹² liter/moles or more and more preferably 10⁸ to 10¹² liter/moles). Any K_{D}value greater than 10⁻⁴ mol/liter (or any K_{A} value lower than 10⁴ M⁻¹) is generally considered to indicate non-specific binding. The K_{D} for biological interactions which are considered meaningful (e.g. specific) are typically in the range of 10⁻¹⁰ M (0.1 nM) to 10⁻⁵ M (10000 nM). The stronger an interaction is, the lower is its K_{D}. Preferably, a binding site on an antibody reagent described herein will bind to the desired antigen with an affinity less than 500 nM, preferably less than 200 nM, more preferably less than 10 nM, such as less than 500 pM. Specific binding of an antibody reagent to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art; as well as other techniques as mentioned herein.

As used herein, the term “specific binding” or “specificity” refers to a chemical interaction between two molecules, compounds, cells and/or particles wherein the first entity binds to the second, target entity with greater specificity and affinity than it binds to a third entity which is a non-target. In some embodiments of any of the aspects, specific binding can refer to an affinity of the first entity for the second target entity which is at least 10 times, at least 50 times, at least 100 times, at least 500 times, at least 1000 times or greater than the affinity for the third nontarget entity. Accordingly, as used herein, “selectively binds” or “specifically binds” refers to the ability of an agent (e.g. an antibody reagent) described herein to bind to a target, such a peptide comprising, e.g. the amino acid sequence of a given antigen, with a K_{D} 10⁻⁵ M (10000 nM) or less, e.g., 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less. For example, if an agent described herein binds to a first peptide comprising the antigen with a K_{D} of 10⁻⁵ M or lower, but not to another randomly selected peptide, then the agent is said to specifically bind the first peptide. Specific binding can be influenced by, for example, the affinity and avidity of the agent and the concentration of the agent. The person of ordinary skill in the art can determine appropriate conditions under which an agent selectively bind the targets using any suitable methods, such as titration of an agent in a suitable cell and/or a peptide binding assay.

As used herein, the term “chimeric”, as used in the context of an antibody, or sequence encoding an antibody refers to immunoglobin molecules characterized by two or more segments or portions derived from different animal species. For example, the variable region of the chimeric antibody is derived from a non-human mammalian antibody, such as murine monoclonal antibody, and the immunoglobin constant region is derived from a human immunoglobin molecule. The variable segments of chimeric antibodies are typically linked to at least a portion of an immunoglobulin constant region (Fc), typically that of a human immunoglobulin. Human constant region DNA sequences can be isolated in accordance with well-known procedures from a variety of human cells, such as immortalized B-cells (WO 87/02671; which is incorporated by reference herein in its entirety). The antibody can contain both light chain and heavy chain constant regions. The heavy chain constant region can include CH1, hinge, CH2, CH3, and, sometimes, CH4 regions. For therapeutic purposes, the CH2 domain can be deleted or omitted. Techniques developed for the production of “chimeric antibodies” are known in the art (see Morrison et al., Proc. Natl. Acad. Sci. 81:851-855 (1984); Neuberger et al., Nature 312:604-608 (1984); Takeda et al., Nature 314:452-454 (1985); which are incorporated by reference herein in their entireties), e.g., by splicing genes from a mouse, or other species, antibody molecule of appropriate antigen specificity together with genes from a human antibody molecule of appropriate biological activity.

As used herein, the term “humanized” refers to an antibody (or fragment thereof, e.g. a light or heavy chain) wherein the CDRs are not human in origin, but the sequence of the remaining sequence of the Ig protein (e.g. the framework regions and constant regions) is human in origin. One of skill in the art is aware of how to humanize a given antibody, see, e.g., U.S. Pat. Nos. 5,585,089; 6,835,823; 6,824,989.

As used herein, the term “engineered” refers to the aspect of having been manipulated by the hand of man. For example, a locus is considered to be “engineered” when two or more sequences, that are not linked together in that order in nature in that locus, are manipulated by the hand of man to be directly linked to one another in the engineered locus. For example, in some embodiments of the present invention, an engineered locus comprises various Ig sequences with a non-native V segment, all of which are found in nature, but are not found in the same locus or are not found in that order in the locus in nature. As is common practice and is understood by those in the art, progeny and copies of an engineered polynucleotide (and/or cells or animals comprising such polynucleotides) are typically still referred to as “engineered” even though the actual manipulation was performed on a prior entity.

As used herein, the term “recombination-defective” refers to a cell (or animal) in which recombination, particularly V(D)J recombination at the IgH and IgL loci cannot occur. Typically, a V(D)J recombination-defective cell is a cell comprising a mutation in a gene encoding a protein that is necessary for V(D)J recombination to occur. Mutations that will cause a cell and/or animal to be V(D)J recombination-defective are known in the art, e.g., RAG2-i-cells are V(D)J recombination defective and mice with such mutations are commercially available (see, e.g., stock number 008449, Jackson Laboratories, Bar Harbor, ME). A further non-limiting example of a V(D)J recombination-defective mutant is RAG1^{-/-}. In some embodiments of any of the aspects, cells can be rendered V(D)J recombination-defective at only one locus, e.g. the IgH locus by, e.g. deleting the germline J_{H} Segments.

As used herein, the term “cassette” refers to a nucleic acid molecule, or a fragment thereof, that can be introduced to a host cell and incorporated into the host cell's genome (e.g. using a cassette-targeting sequence as described elsewhere herein). A cassette can comprise a gene (e.g. an IgH gene), or a fragment thereof, e.g. a V_{H} segment. A cassette can be an isolated nucleotide fragment, e.g. a dsDNA or can be comprised by a vector, e.g. a plasmid, cosmid, and/or viral vector.

As used herein, the term “B cell” refers to lymphocytes that play a role in the humoral immune response and is a component of the adaptive immune system. In this application the expressions “B cell”, “B-cell” and “B lymphocyte” refer to the same cell.

Immature B cells are produced in the bone marrow of most mammals. After reaching the IgM+ immature stage in the bone marrow, these immature B cells migrate to lymphoid organs, where they are referred to as transitional B cells, some of which subsequently differentiating into mature B lymphocytes. B-cell development occurs through several stages, each stage characterized by a change in the genome content at the antibody loci.

Each B cell has a unique receptor protein (referred to as the B-cell receptor (BCR)) on its surface that is able to bind to a unique antigen. The BCR is a membrane-bound immunoglobulin, and it is this molecule that allows to distinguish B cells from other types of lymphocytes, as well as playing a central role in B-cell activation in vivo. Once a B cell encounters its cognate antigen and receives an additional signal from a T helper cell, it can further differentiate into one of two types of B cells (plasma B cells and memory B cells). The B cell may either become one of these cell types directly or it may undergo an intermediate differentiation step, the germinal center reaction, during which the B cell hypermutates the variable region of its immunoglobulin gene (“somatic hypermutation”) and possibly undergoes class switching.

Plasma B cells (also known as plasma cells) are large B cells that have been exposed to an antigen and are producing and secreting large amounts of antibodies. These are short-lived cells and usually undergo apoptosis when the agent that induced the immune response is eliminated. Memory B cells are formed from activated B cells that are specific to an antigen encountered during a primary immune response. These cells are able to live for a long time, and can respond quickly following a second exposure to the same antigen.

As used herein, the term “GC reaction” refers to a process that occurs in the germinal center, during which B cells undergo SHM, memory generation, and/or class/isotype switch. The germinal center (GC) reaction is the basis of T-dependent humoral immunity against foreign pathogens and the ultimate expression of the adaptive immune response. GCs represent a unique collaboration between proliferating antigen-specific B cells, T follicular helper cells, and the specialized follicular dendritic cells that constitutively occupy the central follicular zones of secondary lymphoid organs.

As used herein, the term “somatic hypermutation” or “SHM,” refers to the mutation of a polynucleotide sequence at an Ig locus initiated by, or associated with the action of AID (activation-induced cytidine deaminase) on that polynucleotide sequence. SHM occurs during B cell proliferation and occurs at a mutation rate that is at least 10⁵-10⁶ fold greater than the normal rate of mutation in the genome.

As used herein, the term “stem cell” refers to a cell in an undifferentiated or partially differentiated state that has the property of self-renewal and has the developmental potential to naturally differentiate into a more differentiated cell type, without a specific implied meaning regarding developmental potential (i.e., totipotent, pluripotent, multipotent, etc.). By self-renewal is meant that a stem cell is capable of proliferation and giving rise to more such stem cells, while maintaining its developmental potential. Accordingly, the term “stem cell” refers to any subset of cells that have the developmental potential, under particular circumstances, to differentiate to a more specialized or differentiated phenotype, and which retain the capacity, under certain circumstances, to proliferate without substantially differentiating. The term “somatic stem cell” is used herein to refer to any stem cell derived from non-embryonic tissue, including fetal, juvenile, and adult tissue. Natural somatic stem cells have been isolated from a wide variety of adult tissues including blood, bone marrow, brain, olfactory epithelium, skin, pancreas, skeletal muscle, and cardiac muscle. Exemplary naturally occurring somatic stem cells include, but are not limited to, mesenchymal stem cells and hematopoietic stem cells. In some embodiments of any of the aspects, the stem or progenitor cells can be embryonic stem cells. As used herein, “embryonic stem cells” refers to stem cells derived from tissue formed after fertilization but before the end of gestation, including pre-embryonic tissue (such as, for example, a blastocyst), embryonic tissue, or fetal tissue taken any time during gestation, typically but not necessarily before approximately 10-12 weeks gestation. Most frequently, embryonic stem cells are totipotent cells derived from the early embryo or blastocyst. Embryonic stem cells can be obtained directly from suitable tissue, including, but not limited to human tissue, or from established embryonic cell lines. In one embodiment, embryonic stem cells are obtained as described by Thomson et al. (U.S. Pat. Nos. 5,843,780 and 6,200,806; Science 282:1145, 1998; Curr. Top. Dev. Biol. 38:133 ff, 1998; Proc. Natl. Acad. Sci. U.S.A. 92:7844, 1995 which are incorporated by reference herein in their entirety).

Exemplary stem cells include embryonic stem cells, adult stem cells, pluripotent stem cells, bone marrow stem cells, hematopoietic stem cells, and the like. Descriptions of stem cells, including method for isolating and culturing them, may be found in, among other places, Embryonic Stem Cells, Methods and Protocols, Turksen, ed., Humana Press, 2002; Weisman et al., Annu. Rev. Cell. Dev. Biol. 17:387 403; Pittinger et al., Science, 284:143 47, 1999; Animal Cell Culture, Masters, ed., Oxford University Press, 2000; Jackson et al., PNAS 96(25):14482 86, 1999; Zuk et al., Tissue Engineering, 7:211 228, 2001 (“Zuk et al.”); Atala et al., particularly Chapters 33 41; and U.S. Pat. Nos. 5,559,022, 5,672,346 and 5,827,735. Descriptions of stromal cells, including methods for isolating them, may be found in, among other places, Prockop, Science, 276:71 74, 1997; Theise et al., Hepatology, 31:235 40, 2000; Current Protocols in Cell Biology, Bonifacino et al., eds., John Wiley & Sons, 2000 (including updates through March, 2002); and U.S. Pat. No. 4,963,489.

As used herein, the term “corresponding to” refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments of any of the aspects, specific binding can refer to an affinity of the first entity for the second target entity which is at least 10 times, at least 50 times, at least 100 times, at least 500 times, at least 1000 times or greater than the affinity for the third nontarget entity. A reagent specific for a given target is one that exhibits specific binding for that target under the conditions of the assay being utilized.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A cell comprising at least one of:
   a. an engineered IgH locus comprising a CBE element within the nucleic acid sequence separating the 3′ end of a target V_{H} segment and the 5′ end of the first V_{H} segment which is 3′ of the target V_{H} segment; and/or
   b. an engineered IgL locus comprising at least one of:
      i. a non-functional Cer/Sis sequence within the nucleic acid sequence separating the 3′ end of the 3′-most V_{L} segment and the 5′ end of a J_{L} segment; and
      ii. a CBE element within the nucleic acid sequence separating the 3′ end of a target V_{L} segment and the 5′ end of the first V_{L} segment which is 3′ of the target V_{L}segment.
2. The cell of any of paragraph 1, wherein the CBE element is located 5′ of at least one V segment in the locus.
3. The cell of any of paragraphs 1-2, wherein the CBE element is in the same orientation as the target segment.
4. The cell of any of paragraphs 1-2, wherein the CBE element is in the inverted orientation with respect to the target segment.
5. The cell of any of paragraphs 1-4, wherein the CBE element is located 3′ of the V_{H} recombination signal sequence of the target V segment.
6. The cell of any of paragraphs 1-5, wherein the target V_{H} or V_{L} segment is a non-native, exogenous, or engineered segment.
7. The cell of paragraph 6, wherein the cell is a mouse cell and the target V_{H} or V_{L} segment is a human segment.
8. The cell of any of paragraphs 1-7, further comprising a non-native D_{H}, J_{H}, and/or I_{L} segment.
9. The cell of any of paragraph 8, wherein the non-native D_{H}, J_{H}, or I_{L} segment is a human segment.
10. The cell of any of paragraphs 7-9, wherein the human segment is from a known antibody in need of improvement of affinity or specificity.
11. The cell of any of paragraphs 1-10, wherein the cell is a stem cell embryonic stem cell.
12. The cell of any of paragraphs 1-10, wherein the cell is a murine cell, optionally a murine stem cell or murine embryonic stem cell.
13. The cell of any of paragraphs 1-12, wherein the cell is heterozygous for the engineered IgH and/or IgL locus and the other IgH and/or IgL locus has been engineered to be inactive, wherein the cell will express an IgH and/or IgL chain only from the engineered IgH and/or IgL locus.
14. The cell of any of paragraphs 1-13, further comprising an engineered non-functional IGCR1 sequence in the IgH within the nucleic acid sequence separating the 3′ end of the 3′-most V_{H} segment of the IgH locus and the 5′ end of a D_{H} segment of the IgH locus.
15. The cell of paragraph 14, wherein the non-functional IGCR1 sequence comprises mutated CBE sequences; the CBE sequences of the IGCR1 sequence have been deleted; or the IGCR1 sequence has been deleted from the IgH locus.
16. The cell of any of paragraphs 1-15, further comprising at least one of the following:
   a. an IgL locus with human sequence;
   b. a humanized IgL locus;
   c. a human IgL locus;
   d. an IgH locus with human sequence;
   e. a humanized IgH locus; and
   f. a human IgH locus.
17. The cell of any of paragraphs 1-16, further comprising at least one of the following:
   a. the IgL locus engineered to comprise one J_{L} segment;
   b. an IgH locus engineered to comprise one J_{H} segment; and
   c. an IgH locus engineered to comprise one D_{H} segment;
18. The cell of any of paragraphs 1-17, further comprising a mutation capable of activating, inactivating or modifying genes lead to increased GC antibody maturation responses.
19. The cell of any of paragraphs 1-18, further comprising a cassette targeting sequence in the target segment, which permits the replacement of the target segment.
20. The cell of paragraph 19, wherein the cassette targeting sequence is selected from the group consisting of: an I-SceI meganuclease site; a Cas9/CRISPR target sequence; a Talen target sequence or a recombinase-mediated cassette exchange system.
21. The cell of any of paragraphs 1-20, wherein the cell further comprises an exogenous nucleic acid sequence encoding TdT.
22. The cell of paragraph 21, further comprising a promoter operably linked to the sequence encoding TdT.
23. A genetically engineered mammal comprising the cell of any of paragraphs 1-22.
24. A chimeric genetically engineered mammal comprising two populations of cells, a first population comprising cells which are V(D)J recombination-defective; and a second population comprising cells of any of paragraphs 1-22.
25. The mammal of paragraph 24, wherein the V(D)J recombination-defective cells are RAG2^{-/-} cells.
26. The mammal of any of paragraphs 23-25, wherein the mammal is a mouse.
27. A method of making an antibody, the method comprising the steps of:
injecting a mouse blastocyst with a cell of any of paragraphs 1-22, wherein the cell is a mouse embryonic stem cell;
implanting the mouse blastocyst into a female mouse under conditions suitable to allow maturation of the blastocyst into a genetically engineered mouse;
isolating
   3) an antibody; or
   4) a cell producing an antibody from the genetically engineered mouse.
28. The method of paragraph 27, further comprising a step of immunizing the genetically engineered mouse with a desired target antigen before the isolating step.
29. The method of any of paragraphs 27-28, further comprising a step of producing a monoclonal antibody from at least one cell of the genetically engineered mouse.
30. The method of any of paragraphs 27-29, wherein one or more target segments comprise a non-native V_{L} or V_{H} segment.
31. The method of any of paragraphs 27-29, wherein one or more target segments comprise a non-native V_{L} or V_{H} segment of a known antibody, whereby the known antibody is optimized.
32. An antibody produced by any one of the methods of paragraphs 27-31.
33. A method of identifying a candidate antigen as an antigen that activates a B cell population comprising a V_{H} or V_{L} segment of interest, the method comprising:
   immunizing a mammal of paragraph 23-26, engineered such that a majority of the mammal's peripheral B cells express the V_{H} or V_{L} segment of interest, with the antigen; measuring B cell activation in the mammal; and
   identifying the candidate antigen as an activator of a B cell population comprising the V_{H} or V_{L} segment of interest if the B cell activation in the mammal is increased relative to a reference level.
34. The method of paragraph 33, wherein an increase in B cell activation is an increase in the somatic hypermutation status of the Ig variable region; an increase in the affinity of mature antibodies for the antigen; or an increase in the specificity of mature antibodies for the antigen.
35. A genetically engineered mammal comprising a population of cells comprising at least one of:
   a. an engineered IgH locus comprising at least one of:
      i. a CBE element within the nucleic acid sequence separating the 3′ end of a target V_{H} segment and the 5′ end of the first V_{H} segment which is 3′ of the target V_{H} segment;
      ii. an engineered non-functional IGCR1 sequence in the IgH locus within the nucleic acid sequence separating the 3′ end of the 3′-most V_{H} segment of the IgH locus and the 5′ end of a DH segment of the IgH locus; and/or
   b. an engineered IgL locus comprising at least one of:
      i. a non-functional Cer/Sis sequence within the nucleic acid sequence separating the 3′ end of the 3′-most V_{L} segment and the 5′ end of a J_{L} segment; and
      ii. a CBE element within the nucleic acid sequence separating the 3′ end of a target V_{L} segment and the 5′ end of the first V_{L} segment which is 3′ of the target V_{L}segment;
      whereby V(D)J recombination in the mammal predominantly utilizes the target V_{H} segment and the target V_{L} segment.
36. The mammal of paragraph 35, wherein the target V_{H} segment and/or the target V_{L} segment are human V segments.
37. The mammal of any of paragraphs 35-36, wherein the IgH locus is further engineered to comprise one target D segment and/or one target J_{H} segment.
38. The mammal of any of paragraphs 35-37, wherein the IgL locus is further engineered to comprise one target J_{L} segment.
39. The mammal of any of paragraphs 35-38, wherein the D segment, J_{H} segment, and/or I_{L} segment are human segments.
40. The mammal of any of paragraphs 35-39, wherein the human segments are from a known antibody in need of improvement of affinity or specificity.
41. The mammal of any of paragraphs 35-40, wherein the human segments are highly-utilized human segments.
42. The mammal of any of paragraphs 35-41, wherein the mammal is heterozygous for the engineered IgH and/or IgL locus and the other IgH and/or IgL locus has been engineered to be inactive, wherein the cell will express an IgH and/or IgL chain only from the engineered IgH and/or IgL locus.
43. The mammal of any of paragraphs 35-42, wherein the CBE element is located 5′ of at least one V segment in the locus.
44. The mammal of any of paragraphs 35-43, wherein the CBE element is in the same orientation as the target segment.
45. The mammal of any of paragraphs 35-44, wherein the CBE element is in the inverted orientation with respect to the target segment.
46. The mammal of any of paragraphs 35-45, wherein the CBE element is located 3′ of the V_{H} recombination signal sequence of the target V segment.
47. The mammal of any of paragraphs 35-46, further comprising a mutation capable of activating, inactivating or modifying genes lead to increased GC antibody maturation responses.
48. The mammal of any of paragraphs 35-47, wherein the cell further comprises an exogenous nucleic acid sequence encoding TdT.
49. The mammal of paragraph 48, further comprising a promoter operably linked to the sequence encoding TdT.
50. The mammal of any of paragraphs 35-49, wherein the mammal is a mouse.
51. A set of at least two mammals, wherein each mammal is a mammal of any of paragraphs 35-50, the first mammal comprising a first target V_{H} segment and/or a first target V_{L} segment and each further mammal comprising a further target V_{H} segment and/or a further target V_{L} segment.
52. The set of paragraph 51, wherein each mammal comprises a human target V_{H} segment and a human target V_{L} segment.
53. A method of making an antibody, the method comprising the steps of
   isolating an antibody comprising the one or more target segments from the mammal of any of paragraphs 35-51 or from the set of mammals of paragraphs 51-52, or isolating a cell expressing an antibody comprising the one or more target segments from the mammal of any of paragraphs 35-51 or from the set of mammals of paragraphs 51-52.
54. The method of paragraph 53, further comprising a step of immunizing the genetically engineered mammal with a desired target antigen before the isolating step.
55. An antibody produced by any one of the methods of paragraphs 53-54.
56. A method of making an antibody which is specific for a desired antigen, the method comprising the steps of:
   d) injecting a mouse blastocyst with a cell of any of paragraphs 1-22, wherein the cell is a mouse embryonic stem cell and implanting the mouse blastocyst into a female mouse under conditions suitable to allow maturation of the blastocyst into a genetically engineered mouse or do by RDBC;
   e) immunizing the genetically engineered mouse with the antigen; and
   f) isolating
      3) an antibody specific for the antigen; or
      4) a cell producing an antibody specific for the antigen
   from the genetically engineered mouse.
57. A method of making an antibody which is specific for an antigen, the method comprising the steps of:
   c) immunizing a mammal of any of paragraphs 35-50 or a set of mammals of any of paragraphs 51-52 with the antigen; and
   d) isolating
      3) an antibody specific for the antigen; or
      4) a cell producing an antibody specific for the antigen from the mammal or mammals.
58. The method of any of paragraphs 56-57, further comprising a step of producing a monoclonal antibody from at least one cell of the genetically engineered mouse or mammal.
59. The method of any of paragraphs 56-58, wherein the antibody is humanized.
60. An antibody produced by any one of the methods of paragraphs 56-59.

Some embodiments of the technology described herein can be defined according to any of the following numbered paragraphs:
1. A cell comprising at least one of:
   a. an engineered IgH locus comprising a CBE element within the nucleic acid sequence separating the 3′ end of a target V_{H} segment and the 5′ end of the first V_{H} segment which is 3′ of the target V_{H} segment; and/or
   b. an engineered IgL locus comprising at least one of:
      i. a non-functional Cer/Sis sequence within the nucleic acid sequence separating the 3′ end of the 3′-most V_{L} segment and the 5′ end of a J_{L} segment; and
      ii. a CBE element within the nucleic acid sequence separating the 3′ end of a target V_{L} segment and the 5′ end of the first V_{L} segment which is 3′ of the target V_{L}segment.
2. The cell of any of paragraph 1, wherein the CBE element is located 5′ of at least one V segment in the locus.
3. The cell of any of paragraphs 1-2, wherein the CBE element is in the same orientation as the target segment.
4. The cell of any of paragraphs 1-2, wherein the CBE element is in the inverted orientation with respect to the target segment.
5. The cell of any of paragraphs 1-4, wherein the CBE element is located 3′ of the V_{H} recombination signal sequence of the target V segment.
6. The cell of any of paragraphs 1-5, wherein the target V_{H} or V_{L} segment is a non-native, exogenous, or engineered segment.
7. The cell of paragraph 6, wherein the cell is a mouse cell and the target V_{H} or V_{L} segment is a human segment.
8. The cell of any of paragraphs 1-7, further comprising a non-native D_{H}, J_{H}, and/or I_{L} segment.
9. The cell of any of paragraph 8, wherein the non-native D_{H}, J_{H}, or I_{L} segment is a human segment.
10. The cell of any of paragraphs 7-9, wherein the human segment is from a known antibody in need of improvement of affinity or specificity.
11. The cell of any of paragraphs 1-10, wherein the cell is a stem cell embryonic stem cell.
12. The cell of any of paragraphs 1-10, wherein the cell is a murine cell, optionally a murine stem cell or murine embryonic stem cell.
13. The cell of any of paragraphs 1-12, wherein the cell is heterozygous for the engineered IgH and/or IgL locus and the other IgH and/or IgL locus has been engineered to be inactive, wherein the cell will express an IgH and/or IgL chain only from the engineered IgH and/or IgL locus.
14. The cell of any of paragraphs 1-13, further comprising
   an engineered non-functional IGCR1 sequence in the IgH within the nucleic acid sequence separating the 3′ end of the 3′-most V_{H} segment of the IgH locus and the 5′ end of a D_{H} segment of the IgH locus.
15. The cell of paragraph 14, wherein the non-functional IGCR1 sequence comprises mutated CBE sequences; the CBE sequences of the IGCR1 sequence have been deleted; or the IGCR1 sequence has been deleted from the IgH locus.
16. The cell of any of paragraphs 1-15, further comprising at least one of the following:
   a. an IgL locus with human sequence;
   b. a humanized IgL locus;
   c. a human IgL locus;
   d. an IgH locus with human sequence;
   e. a humanized IgH locus; and
   f. a human IgH locus.
17. The cell of any of paragraphs 1-16, further comprising at least one of the following:
   a. the engineered IgH locus further engineered to comprise only one V_{H} segment;
   b. the engineered IgL locus further engineered to comprise only one V_{L} segment;
   c. the IgL locus engineered to comprise one J_{L} segment;
   d. an IgH locus engineered to comprise one J_{H} segment; and
   e. an IgH locus engineered to comprise one D_{H} segment.
18. The cell of any of paragraphs 1-17, further comprising a mutation capable of activating, inactivating or modifying genes lead to increased GC antibody maturation responses.
19. The cell of any of paragraphs 1-18, further comprising a cassette targeting sequence in the target segment, which permits the replacement of the target segment.
20. The cell of paragraph 19, wherein the cassette targeting sequence is selected from the group consisting of:
   an I-SceI meganuclease site; a Cas9/CRISPR target sequence; a Talen target sequence or a recombinase-mediated cassette exchange system.
21. The cell of any of paragraphs 1-20, wherein the cell further comprises an exogenous nucleic acid sequence encoding TdT.
22. The cell of paragraph 21, further comprising a promoter operably linked to the sequence encoding TdT.
23. A genetically engineered mammal comprising the cell of any of paragraphs 1-22.
24. A chimeric genetically engineered mammal comprising two populations of cells,
   a first population comprising cells which are V(D)J recombination-defective; and
   a second population comprising cells of any of paragraphs 1-22.
25. The mammal of paragraph 24, wherein the V(D)J recombination-defective cells are RAG2⁻¹-cells.
26. The mammal of any of paragraphs 23-25, wherein the mammal is a mouse.
27. A method of making an antibody, the method comprising the steps of:
   injecting a mouse blastocyst with a cell of any of paragraphs 1-22, wherein the cell is a mouse embryonic stem cell;
   implanting the mouse blastocyst into a female mouse under conditions suitable to allow maturation of the blastocyst into a genetically engineered mouse;
   isolating
      5) an antibody; or
      6) a cell producing an antibody
   from the genetically engineered mouse.
28. The method of paragraph 27, further comprising a step of immunizing the genetically engineered mouse with a desired target antigen before the isolating step.
29. The method of any of paragraphs 27-28, further comprising a step of producing a monoclonal antibody from at least one cell of the genetically engineered mouse.
30. The method of any of paragraphs 27-29, wherein one or more target segments comprise a non-native V_{L} or V_{H} segment.
31. The method of any of paragraphs 27-29, wherein one or more target segments comprise a non-native V_{L} or V_{H} segment of a known antibody, whereby the known antibody is optimized.
32. An antibody produced by any one of the methods of paragraphs 27-31.
33. A method of identifying a candidate antigen as an antigen that activates a B cell population comprising a V_{H} or V_{L} segment of interest, the method comprising:
   immunizing a mammal of paragraph 23-26, engineered such that a majority of the mammal's peripheral B cells express the V_{H} or V_{L} segment of interest, with the antigen; measuring B cell activation in the mammal; and
   identifying the candidate antigen as an activator of a B cell population comprising the V_{H} or V_{L} segment of interest if the B cell activation in the mammal is increased relative to a reference level.
34. The method of paragraph 33, wherein an increase in B cell activation is an increase in the somatic hypermutation status of the Ig variable region; an increase in the affinity of mature antibodies for the antigen; or an increase in the specificity of mature antibodies for the antigen.
35. A genetically engineered mammal comprising a population of cells comprising at least one of:
   a. an engineered IgH locus comprising at least one of:
      i. a CBE element within the nucleic acid sequence separating the 3′ end of a target V_{H} segment and the 5′ end of the first V_{H} segment which is 3′ of the target V_{H}segment;
      ii. an engineered non-functional IGCR1 sequence in the IgH locus within the nucleic acid sequence separating the 3′ end of the 3′-most V_{H} segment of the IgH locus and the 5′ end of a D_{H} segment of the IgH locus; and/or
   b. an engineered IgL locus comprising at least one of:
      i. a non-functional Cer/Sis sequence within the nucleic acid sequence separating the 3′ end of the 3′-most V_{L} segment and the 5′ end of a J_{L} segment; and
      ii. a CBE element within the nucleic acid sequence separating the 3′ end of a target V_{L} segment and the 5′ end of the first V_{L} segment which is 3′ of the target V_{L}segment;
      whereby V(D)J recombination in the mammal predominantly utilizes the target V_{H}segment and the target V_{L} segment.
36. The mammal of paragraph 35, wherein the target V_{H} segment and/or the target V_{L} segment are human V segments.
37. The mammal of any of paragraphs 35-36, wherein the IgH locus is further engineered to comprise one target D segment and/or one target J_{H} segment.
38. The mammal of any of paragraphs 35-37, wherein the IgL locus is further engineered to comprise one target J_{L} segment.
39. The mammal of any of paragraphs 35-38, wherein the D segment, J_{H} segment, and/or J_{L} segment are human segments.
40. The mammal of any of paragraphs 35-39, wherein the human segments are from a known antibody in need of improvement of affinity or specificity.
41. The mammal of any of paragraphs 35-40, wherein the human segments are highly-utilized human segments.
42. The mammal of any of paragraphs 35-41, wherein the mammal is heterozygous for the engineered IgH and/or IgL locus and the other IgH and/or IgL locus has been engineered to be inactive, wherein the cell will express an IgH and/or IgL chain only from the engineered IgH and/or IgL locus.
43. The mammal of any of paragraphs 35-42, wherein the CBE element is located 5′ of at least one V segment in the locus.
44. The mammal of any of paragraphs 35-43, wherein the CBE element is in the same orientation as the target segment.
45. The mammal of any of paragraphs 35-44, wherein the CBE element is in the inverted orientation with respect to the target segment.
46. The mammal of any of paragraphs 35-45, wherein the CBE element is located 3′ of the V_{H} recombination signal sequence of the target V segment.
47. The mammal of any of paragraphs 35-46, further comprising a mutation capable of activating, inactivating or modifying genes lead to increased GC antibody maturation responses.
48. The mammal of any of paragraphs 35-47, wherein the cell further comprises an exogenous nucleic acid sequence encoding TdT.
49. The mammal of paragraph 48, further comprising a promoter operably linked to the sequence encoding TdT.
50. The mammal of any of paragraphs 35-49, wherein the mammal is a mouse.
51. A set of at least two mammals, wherein each mammal is a mammal of any of paragraphs 35-50, the first mammal comprising a first target V_{H} segment and/or a first target V_{L} segment and each further mammal comprising a further target V_{H} segment and/or a further target V_{L} segment.
52. The set of paragraph 51, wherein each mammal comprises a human target V_{H} segment and a human target V_{L} segment.
53. A method of making an antibody, the method comprising the steps of: isolating an antibody comprising the one or more target segments from the mammal of any of paragraphs 35-51 or from the set of mammals of paragraphs 51-52, or isolating a cell expressing an antibody comprising the one or more target segments from the mammal of any of paragraphs 35-51 or from the set of mammals of paragraphs 51-52.
54. The method of paragraph 53, further comprising a step of immunizing the genetically engineered mammal with a desired target antigen before the isolating step.
55. An antibody produced by any one of the methods of paragraphs 53-54.
56. A method of making an antibody which is specific for a desired antigen, the method comprising the steps of:
   a) injecting a mouse blastocyst with a cell of any of paragraphs 1-22, wherein the cell is a mouse embryonic stem cell and implanting the mouse blastocyst into a female mouse under conditions suitable to allow maturation of the blastocyst into a genetically engineered mouse or do by RDBC;
   b) immunizing the genetically engineered mouse with the antigen; and
   c) isolating
      1) an antibody specific for the antigen; or
      2) a cell producing an antibody specific for the antigen
   from the genetically engineered mouse.
57. A method of making an antibody which is specific for an antigen, the method comprising the steps of:
   a) immunizing a mammal of any of paragraphs 35-50 or a set of mammals of any of paragraphs 51-52 with the antigen; and
   b) isolating
      1) an antibody specific for the antigen; or
      2) a cell producing an antibody specific for the antigen
   from the mammal or mammals.
58. The method of any of paragraphs 56-57, further comprising a step of producing a monoclonal antibody from at least one cell of the genetically engineered mouse or mammal.
59. The method of any of paragraphs 56-58, wherein the antibody is humanized.
60. An antibody produced by any one of the methods of paragraphs 56-59.

### EXAMPLES

### Example 1: A Set of Mice Rearranging Individual Human V_{H} Segments to Provide a More Human Like Repertoire to Discover New Therapeutic Human Antibodies

We have previously described a mouse model in which the most proximal mouse V_{H} is replaced with a desired human V_{H} in the context of deletion of the IGCR1 regulatory element. In such models, the inserted human V_{H} is rearranged very frequently with either mouse D and J_{H} or with inserted human DJ_{H} to generate a vast repertoire B cells most of which express the inserted human V_{H} in association with unique antigen binding CDR3 that assembled via diversification processes that occur during assembly of V_{H}DJ_{H} junctions ((Tian et al., 2016)).

We have taken this approach to take apart the V_{H}, D, and J_{H} segments of an existing anti-PD1 antibody described by BMS (Korman et al., U.S. Pat. No. 8,008,449 B2); similar antibodies have been widely used for cancer immunotherapy. These anti-PD1 IgH chains gene segments are employed form one of these antibodies in our rearrangement model to generate mice that express a vast array of IgH precursors from that antibody, with novel CDR3 antigen contact region (due to V(D)J recombination junctional diversification). We also made such antibodies using the PD1 IgH V segment described above for rearrangement to mouse Ds and JHs or the DJH of the original anti-PD1 antibody. All of these mice also expressed a fixed IgL chain from the original anti-PD1 antibody. Upon immunization with PD1, antibodies obtained from this mouse model had many novel humanized PD1 antibodies relative to their precursor and two therapeutically employed anti-PD1 antibodies. These novel antibodies have similar affinity for PD-1 as the high affinity antibody from which they were derived but altered overall binding characteristics and/or epitopes and significant sequence differences in CDR3 and other parts of the variable region sequences.

The largest impact on the diversity of BCR repertoires derives from CDR3, especially of the Ig heavy chain. There are a huge number of potential CDR3 sequences that can be generated in humans and mice, numbers greatly exceed the number of lymphocytes in mice or humans. Thus, the total diversity of the antibody repertoire is largely limited by the number of B cells. Humans have orders of magnitude more B cells than mice. For this reason, mice can only express a tiny fraction of the human CDR3 repertoires for a given antibody precursor in naive B cells. Thus, the success of the models described above relative to that of existing humanized antibody mouse models is based in large part on making mice that can express a larger, more human-like CDR3 repertoire for a one given set of human antibody IgH and IgL chains versus making antibodies from 100 s of IgH and IgL V(D)J combinations. Based on our new Ig repertoire sequencing method it has been found that humans tend to predominately use a subset of their IgH and IgL chains in their naïve repertoires. Therefore, described herein are certain mice, each of which rearranges a given highly utilized human V_{H} segment and human V_{L} segment. The mice described herein can be used for immunization of a desired target antigen to discover new humanized antibodies which can then be further optimized by the optimization methods outlined herein and in US Patent Publication 2016/0374320; which is incorporated by reference herein in its entirety.

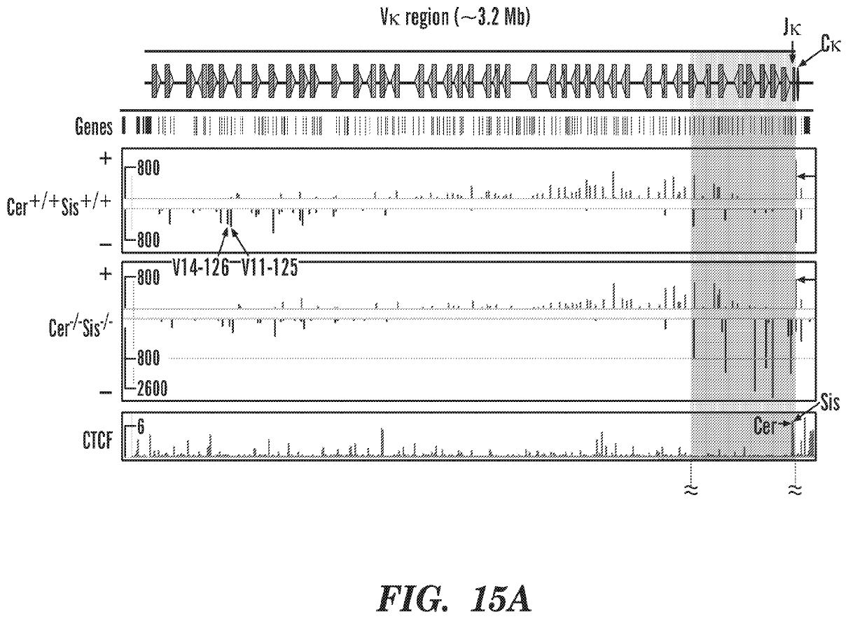

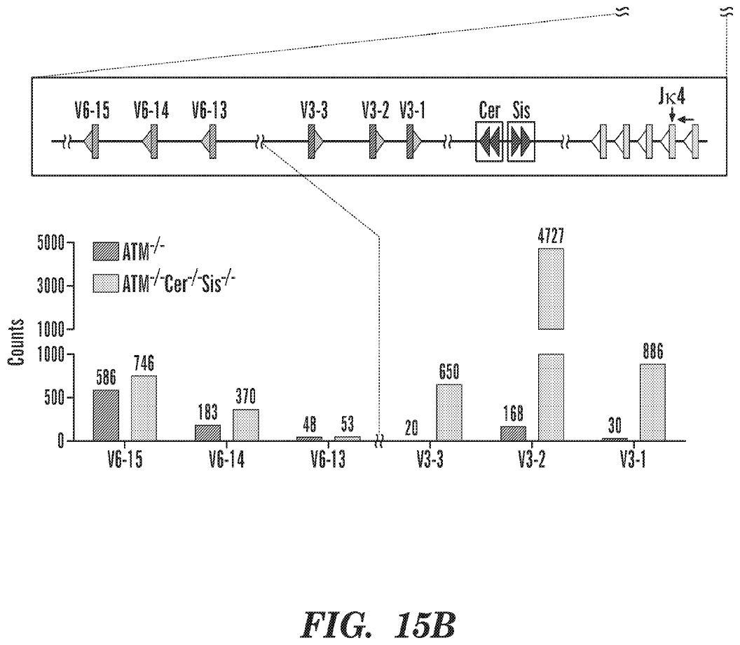

To complement the Ig heavy chain diversification described above, also described herein are mice that will dominantly rearrange a specific IgL chain V segment based on findings that deletion of an element named Cer/Sis leads to increased proximal Vκ light chain utilization, similar to the effects of IGCR1 deletion in IgH. However, this effect is not as predominant as in IgH, likely because the IgH proximal V_{H} segments have an additional element, termed a CBE, that enforces their over-utilization in the absence of IGCR1 (Jain et al., Cell in press; see also appended Igκ rearrangment data; and FIGS. 15A-15B). Thus, for this IgL rearranging model a CBE is also added just downstream from the inserted human VL segment to enforce its dominant rearrangement.

TdT ectopic expression can also be introduced in into these mice as repertoire sequencing observations confirm the earlier speculation (Alt and Baltimore, 1982) that mouse IgL repertoire diversity is much less in mice that humans due to lack of TdT expression in mouse pro-B cells undergoing IgL rearrangement. These modifications will yield a mouse model that can express a much more human-like diverse repertoire of a selected human IgL VJ exons.

These IgH and IgL rearranging mice can be bred to make rearranging models that will each express large, more human-like repertoires of a given pair of rearranging IgH and IgL chains than conventional humanized mice with complete Ig loci that are now used for humanized antibody discovery. Immunization of this set of mice can permit the discovery of superior, novel humanized therapeutic antibodies, which can be further improved, if necessary, by our current antibody optimization mouse model.

Specifically contemplated herein is the immunization of a prototype of this new discovery model with PD-1. Several additional boosts can be performed, prior to isolation and characterization of high affinity humanized anti-PD1 antibodies.

Further contemplated herein is a second model, based on the original anti-PD-1 model described above. The model comprises mice engineered by replacing their VHs and J_{H}S, e.g., via the now standard Cas-9gRNA Zygote injection/electroporation methodology. See, e.g., Wang et al. Cell. 2013; 153(4):910-8; Yang et al. Cell. 2013; 154(6):1370-9; Yasue et al. Scientific reports. 2014; 4:5705; Hashimoto et al. Developmental biology. 2016; 418(1):1-9; and Wang et al. BioTechniques. 2015; 59(4):201-2, 4, 6-8.

### REFERENCES

Alt, F. W., and Baltimore, D. (1982). Joining of immunoglobulin heavy chain gene segments: implications from a chromosome with evidence of three D-J_{H} fusions. Proceedings of the National Academy of Sciences of the United States of America 79, 4118-4122.
Tian, M., Cheng, C., Chen, X., Duan, H., Cheng, H.L., Dao, M., Sheng, Z., Kimble, M., Wang, L., Lin, S., et al. (2016). Induction of HIV Neutralizing Antibody Lineages in Mice with Diverse Precursor Repertoires. Cell 166, 1471-1484.ei418.

### Example 2

The invention in brief is the discovery that insertion of a CTCF-binding element (CBE) adjacent to an antibody variable region gene segment can greatly increase its rearrangement frequency. This invention permits the generation of mouse models focused on rearrangement of particular IgH and IgL to make a more human like repertoires of antibody precursors from which to selectively generate high affinity humanized antibodies.

Antigen-binding variable region exons of antibody molecules are assembled from germline V, D and J gene segments by a V(D)J recombination process. This process is initiated by the RAG endonuclease within a chromosomal V(D)J recombination center (RC) by cleaving between paired gene segments and flanking recombination signal sequences (RSSs). The mouse heavy chain locus (Igh) harbors a high density of sites that bind a ubiquitously expressed architectural protein called CTCF that facilitates chromosomal looping and plays an important role in organizing the genome into topologically associated domains that regulate various physiological processes. In the Igh locus, the vast majority of these CTCF-binding elements (CBEs) are spread across the VH domain. CBE organization is particularly striking in the DH-proximal part of the VH domain where CBEs lie immediately downstream of the RSS of functional VH segments.

It was found that mutation of the CBE that lies next to the most DH-proximal functional VH segment, VH81X, which is also the most highly rearranging VH segment in mouse progenitor cells, results in 50-100 fold reduction in VH81X utilization while rearrangement of a few immediately upstream VHs is increased. Similarly, mutation of the CBE flanking the next upstream VH segment resulted in a 100-fold reduction in the utilization of its associated VH segment while rearrangement of a few immediately upstream VHs increased.

Although VH81X is the most highly utilized VH segment in progenitor B cells, the most DH-proximal VH segment is an infrequently utilized pseudogene called VH5-1 that is flanked by a non-functional vestigial CBE. Restoration of this CBE converted VH5-1 into the most highly utilized VH while rearrangement of VH81X and other frequently rearranging upstream VHs was significantly reduced. Thus, the presence of a CBE tremendously enhances the recombination potential of the associated VH by making it accessible to RAG that linearly scans chromatin for its substrates. This scanning process initiates from the downstream RC and is likely mediated by loop extrusion during which VH-associated CBEs stabilize interactions of DH-proximal VHs first encountered by the RC, thereby promoting their dominant rearrangement.

A similar RAG scanning process operates in the mouse Igκ locus that encodes the antibody Iκ light chain (see, e.g., FIGS. 15A-15B). When an element called Cer/Sis that lies between the Vκ and Jκ segments is removed, RAG scans into the proximal Vκs resulting in their increased utilization, although not nearly to the level of dominance found for proximal IgH VHs during scanning. In this regard, the majority of Vκs are not flanked by a CBE. Therefore, similar to the effect of restoring VH5-1-CBE, insertion of CBE downstream of a proximal Vκ segment can result in similar dominant rearrangement of the associated Vκ. This effect permits mouse models that dominantly rearrangement proximal Vκ sequences.

This approach can be tapped to generate diverse antibody repertoires using any V segment of choice simply by replacing the most proximal VH and/or Vκ segment with a corresponding human V segment of interest and retaining or inserting a CBE next to it. Also contemplated herein is the combination of this model with existing IgH models to make a fully VH and Vκ rearranging model that can be used to optimize the affinity of existing humanized therapeutic antibodies and to also discover new ones based on, e.g., V(D)J junctional regions making the major contribution to antigen binding with SHM followed by selection maturing the complete V(D)J exon (CDRs 1, 2 and 3)

### Example 3: CTCF-Binding Elements Mediate Accessibility of RAG Substrates During Chromatin Scanning

RAG endonuclease initiates antibody heavy chain variable region exon assembly from V, D, and J segments within a chromosomal V(D)J recombination center (RC) by cleaving between paired gene segments and flanking recombination signal sequences (RSSs). The IGCR1 control region promotes DJH intermediate formation by isolating Ds, JHs, and RC from upstream VHs in a chromatin loop anchored by CTCF-binding elements (“CBEs”). How VHs access the DJHRC for VH to DJH rearrangement was previously unknown. It is described herein that CBEs immediately downstream of frequently rearranged VH-RSSs increase recombination potential of their associated VH far beyond that provided by RSSs alone. This CBE activity becomes particularly striking upon IGCR1 inactivation, which allows RAG, likely via loop extrusion, to linearly scan chromatin far upstream. VH-associated CBEs stabilize interactions of D-proximal VHs first encountered by the DJHRC during linear RAG scanning and, thereby, promote dominant rearrangement of these VHs by an unanticipated chromatin accessibility-enhancing CBE function.

Exons encoding immunoglobulin (Ig) or T cell receptor variable regions are assembled from V, D, and J gene segments during B and T lymphocyte development. V(D)J recombination is initiated by RAG1/RAG2 endonuclease (RAG), which introduces DNA double-stranded breaks (DSBs) between a pair of V, D, and J coding segments and flanking recombination signal sequences (RSSs) (Teng and Schatz, 2015). RSSs consist of a conserved heptamer, closely related to the canonical 5′-CACAGTG-3′ sequence, and a less-conserved nonamer separated by 12 (12RSS) or 23 (23RSS) base pair (bp) spacers. Physiological RAG cleavage requires RSSs and is restricted to paired coding segments flanked, respectively, by 12RSSs and 23RSSs (Teng and Schatz, 2015). RAG binds paired RSSs as a Y-shaped heterodimer (Kim et al., 2015; Ru et al., 2015), with cleavage occurring adjacent to heptamer CACs. Cleaved coding and RSS ends reside in a RAG post-cleavage synaptic complex prior to fusion of RSS ends and coding ends, respectively, by non-homologous DSB end-joining (Alt et al., 2013).

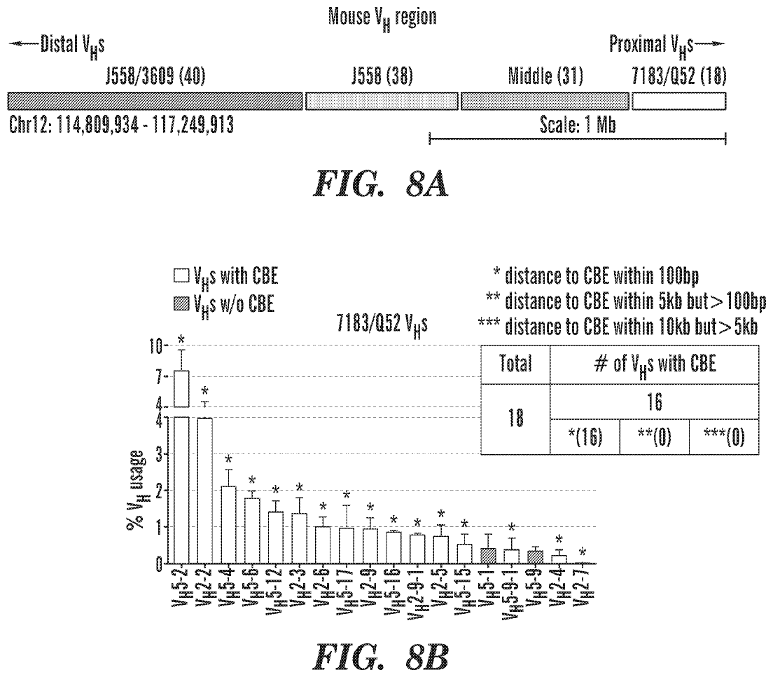

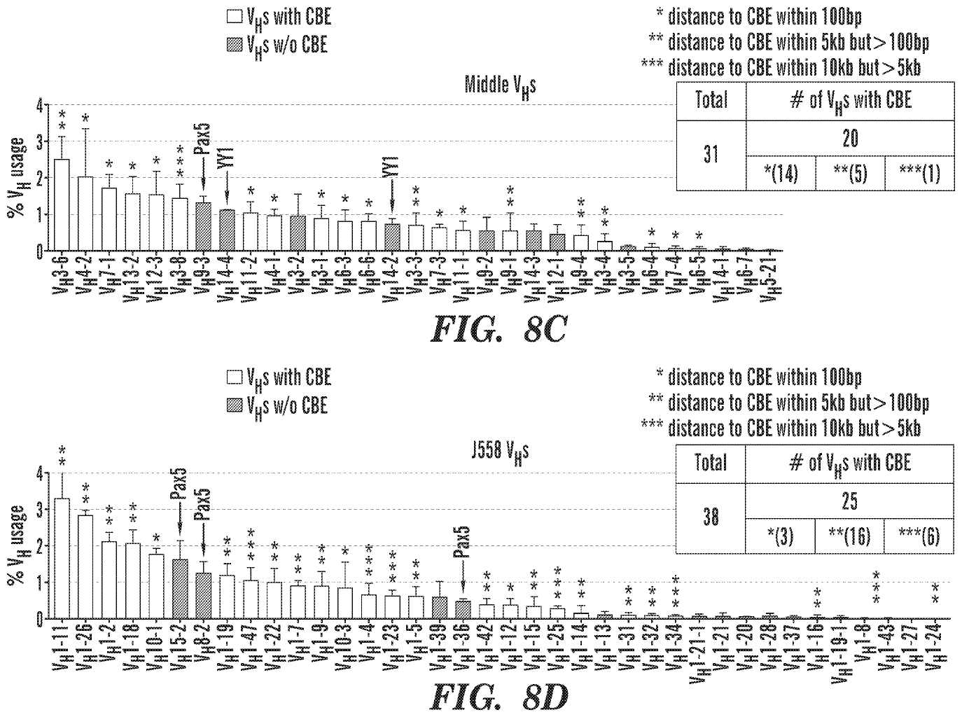

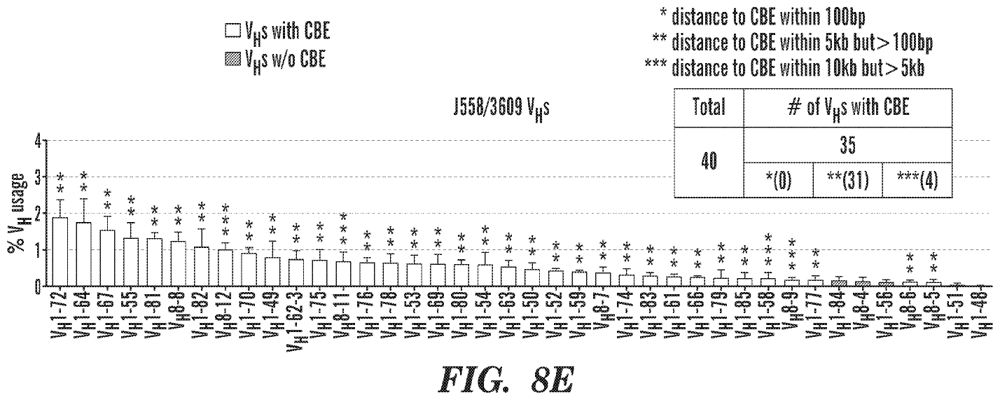

The mouse Ig heavy chain locus (Igh) spans 2.7 megabases (Mb), with more than 100 VHs flanked by 23RSSs embedded in the 2.4 Mb distal portion; 13 Ds flanked on each side by a 12RSS located in a region starting 100 kb downstream of the D-proximal V_{H} (V_{H}5-2; commonly termed “V_{H}81X”), and 4 JHs flanked by 23RSSs lying just downstream of the Ds (Alt et al., 2013; FIGS. 1A and 8A). Igh V(D)J recombination is ordered, with Ds joining on their downstream side to JHs before VHs join to the upstream side of the DJH intermediate (Alt et al., 2013). D to J_{H} joining initiates after RAG is recruited to a nascent V(D)J recombination center (“nRC”) to form an active V(D)J recombination center (RC) around the Igh intronic enhancer (iEμ i), JHs, and proximal DHQ52 (Teng and Schatz, 2015). Upon formation of DJH intermediates, VHs must enter a newly established DJHRC for joining. In this regard, Igh locus contraction brings VHs into closer physical proximity to the DJHRC, allowing utilization of VHs from across the V_{H} domain (Bossen et al., 2012; Ebert et al., 2015; Proudhon et al., 2015). Following locus contraction, diffusion-related mechanisms contribute to V_{H} incorporation into the DJHRC (Lucas et al., 2014). Yet, diffusion access alone may not explain reproducible variations in relative utilization of individual VHs (Lin et al., 2016; Bolland et al., 2016).

V(D)J recombination is regulated to maintain specificity and diversity of antigen receptor repertoires by modulating chromatin accessibility of particular Ig or TCR loci, or regions of these loci, for V(D)J recombination (Yancopoulos et al., 1986; Alt et al., 2013). Accessibility regulation was proposed based on robust transcription of distal VHs before rearrangement (Yancopoulos and Alt, 1985) and correlated with various epigenetic modifications (Alt et al., 2013). In this regard, germline transcription and active chromatin modifications in the nRC recruit RAG1 and RAG2 to form the active RC (Teng and Schatz, 2015). Genome organization alterations also positively impact V_{H} “accessibility” by bringing distal VHs into closer physical proximity to the DJHRC via Igh locus contraction (Bossen et al., 2012). Conversely, the intergenic control region 1 (IGCR1) in the V_{H} to D interval plays a negative, insulating role with respect to proximal V_{H} accessibility (Guo et al., 2011). IGCR1 function relies on two CTCF looping factor binding elements (“CBEs”) that contribute to sequestering Ds, JHs and RC within a chromatin domain that excludes proximal VHs; thereby, mediating ordered D to J_{H} recombination and preventing proximal V_{H} over-utilization (Guo et al., 2011; Lin et al; 2015; Hu et al., 2015).

Eukaryotic genomes are organized into Mb or sub-Mb topologically associated domains (TADs) (Dixon et al., 2012; Nora et al., 2012) that often include contact loops anchored by pairs of convergent CBEs bound by CTCF in association with cohesin (Phillips-Cremins et al., 2013; Rao et al., 2014). In this regard, CTCF binds CBEs in an orientation-dependent fashion. Ability to recognize widely separated convergent CBEs may involve cohesin, or other factors, that progressively extrude a growing chromatin loop that is fixed into a domain upon reaching convergent CTCF-bound loop anchors (Sanbom et al., 2015; Nichols and Corces, 2015; Fudenberg et al., 2016; Dekker and Mimy, 2016). In mammalian cells, CBEs, TADs and/or loop domains have been implicated in regulation of various physiological processes (Dekker and Mimy, 2016; Merkenschlager and Nora, 2016; Hnisz et al., 2016), with convergent CBE-based loop organization implicated as critical for such regulation in some cases (Sanbom et al., 2015; Guo et al., 2015; de Wit et al., 2015; Ruiz-Velasco et al., 2017).

RAG can explore directionally from an initiating physiological or ectopically introduced RC for Mb distances within convergent CBE-based contact chromatin loop domains genome-wide (Hu et al., 2015). During such exploration, RAG uses RSSs in convergent orientation, including cryptic RSSs as simple as a CAC, for cleavage and joining to a canonical RSS in the RC (Hu et al., 2015; Zhao et a., 2016). This long-range directional RAG activity is impeded upon encounter of cohesin-bound convergent CBE pairs and potentially by other blockages that create chromatin sub-domains within loops (Hu et al., 2015; Zhao et al., 2016). The directionality and linearity of RAG activity across these domains implicated one-dimensional RAG tracking (Hu et al., 2015). Directional RAG tracking also occurs upstream of the DJHRC to IGCR1 (Hu et al., 2015). IGCR1 deletion extends this recombination tracking domain directionally upstream from the DJHRC to the proximal VHs, coupled with dramatically increased proximal VH to DJH joining, most dominantly VH81X (Hu et al., 2015). However, the nature of the tracked substrate and factors that drive RAG tracking remained speculative.

The mouse Igh harbors a high density of CBEs (Degner et al., 2011). Ten clustered CBEs (“3′CBEs”) lie at the downstream Igh boundary in convergent orientation to more than 100 CBEs embedded across the VH domain (Proudhon et al., 2015). VH CBEs are spread throughout the VH domain and, particularly for more proximal VHs, often found immediately downstream of VH RSSs (Choi et al., 2013; Bolland et al., 2016). Notably, VH CBEs and 3′CBEs are in convergent orientation with each other and with, respectively, the upstream and downstream IGCR1 CBEs (Guo et al., 2011). The striking number and organization of the CBEs across the VH portion of Igh has led to speculation of potential positive or negative VH CBE roles in Igh V(D)J recombination (Bossen et al., 2012; Guo et al., 2011; Benner et al., 2015; Degner et al., 2011; Lin et al., 2015). Our current studies reveal the function of proximal VH CBEs and provide new insights into the RAG tracking mechanism.

### Results

The VH81X-CBE Greatly Augments VH81X Utilization in Primary Pro-B Cells

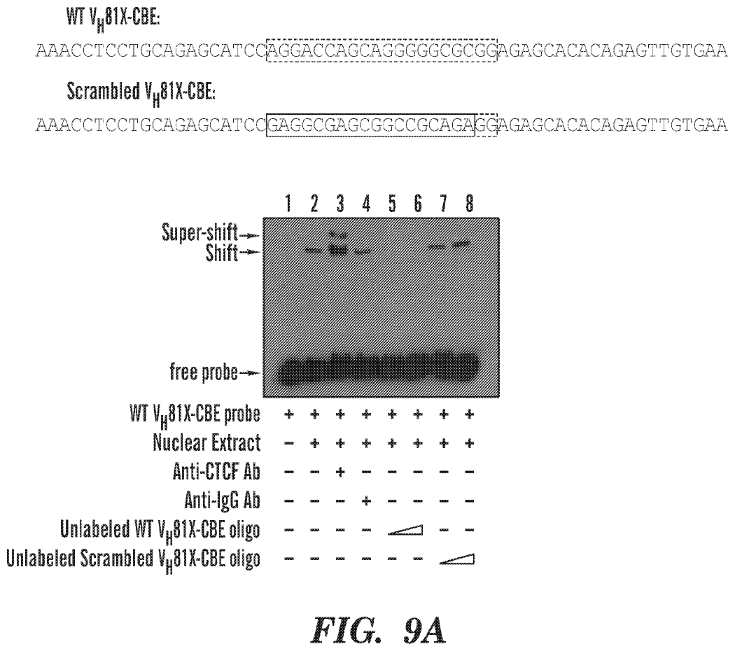

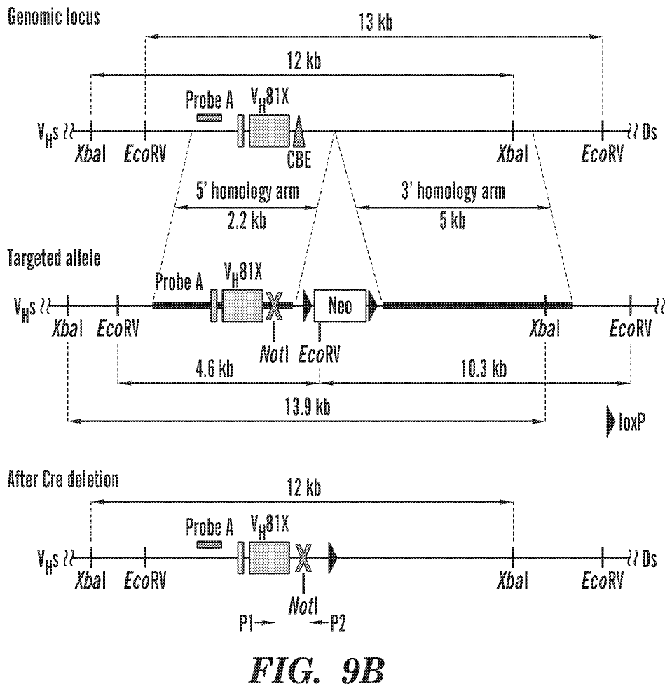

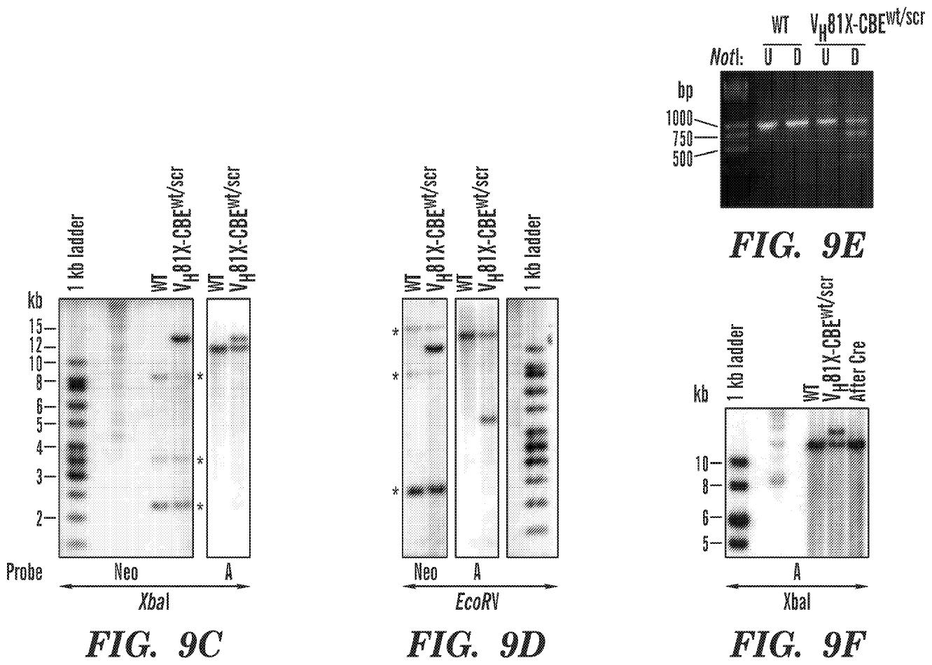

To examine potential functions of the CBE immediately downstream of VH81X, 129SV ES cells were generated in which the 18-bp VH81X-CBE sequence was replaced with a scrambled sequence that does not bind CTCF (FIGS. 1A, 1B and 9A-9F). This mutation, referred to as “VH81X-CBEscr”, was introduced into the 129SV mouse germline. VH to DJH recombination occurs in progenitor (pro) B cells in the bone marrow (BM), in which overall VH utilization frequency provides an index of relative rearrangement frequency (Lin et al., 2016; Bolland et al., 2016). To quantify utilization of each of the 100 s of distinct VHs across the 129SV mouse Igh locus in B220+CD43highIgM-BM pro-B cells, highly sensitive high-throughput genome-wide translocation sequencing (HTGTS)-based V(D)J repertoire sequencing (“HTGTS-Rep-Seq”; Hu et al., 2015; Lin et al., 2016) was employed using a JH4-coding end primer as bait. For these analyses assays were performed on four independent VH81X-CBEscr homozygous mutant mice (VH81X-CBEscr/scr mice) and three wild-type (WT) controls. For statistical analyses, data from each library was normalized to 10,000 total VDJH junctions, and similarly normalized data from other experiments described below (See STAR Methods).

VH81X is the most highly utilized VH in WT 129SV mouse pro-B cells being used in about 10% of total VDJH junctions, with VH2-2, which lies approximately 10 kb immediately upstream, being the second most highly utilized at 6% of junctions (FIGS. 1C and 1D; Table 1). The three proximal VHs immediately upstream of VH2-2 also are highly utilized with frequencies of 3%, 2.2%, and 1.6%, respectively (FIGS. 1C and 1D; Table 1). Even though WT pro-B cells have undergone locus contraction (Medvedovic et al., 2013), only a few of the most highly used VHs further upstream approach the 2-3% utilization range and many are utilized far less frequently (FIG. 1C). As noted previously (Yancopoulos et al., 1984), the VH5-1 pseudo-gene 5 kb downstream of VH81X is infrequently utilized (about 0.4%), despite its canonical RSS (FIGS. 1C and 1D; Table S1). Strikingly, in VH81X-CBEscr/scr mutant mice, VH81X utilization was reduced approximately 50-fold to 0.2% of junctions with a concomitant increase in utilization of VH2-2 and next three upstream VHs (FIGS. 1C and 1D; Table 1). However, there were no significant effects on utilization of further upstream VHs or the downstream VH5-1 (FIGS. 1C and 1D; Table 1). Thus, the VH81X-CBE is required to promote VH81X rearrangement in mouse pro-B cells; and, in its absence, utilization of the upstream VH2-2 doubles to make it the most utilized VH.

VH81X-CBE Greatly Augments VH81X to DJH Rearrangement in a v-Abl Pro-B Cell Line

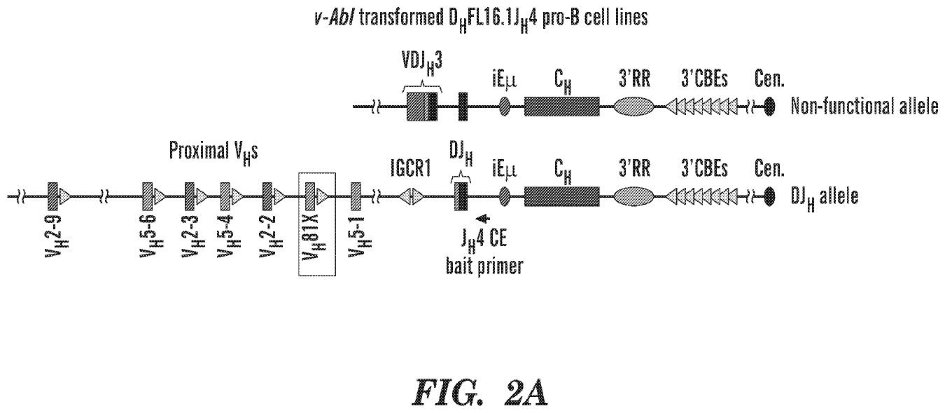

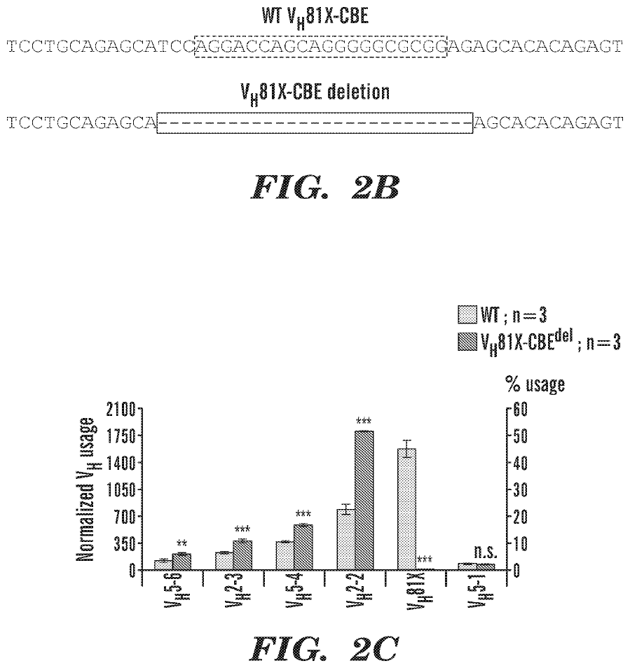

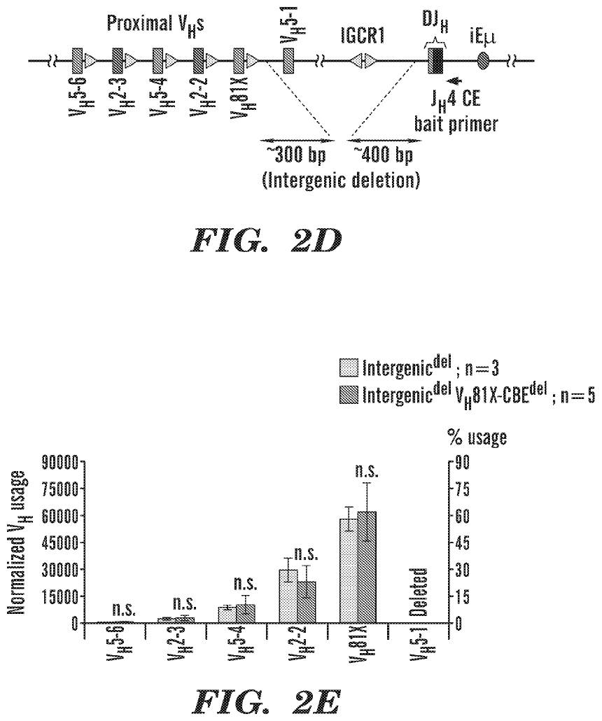

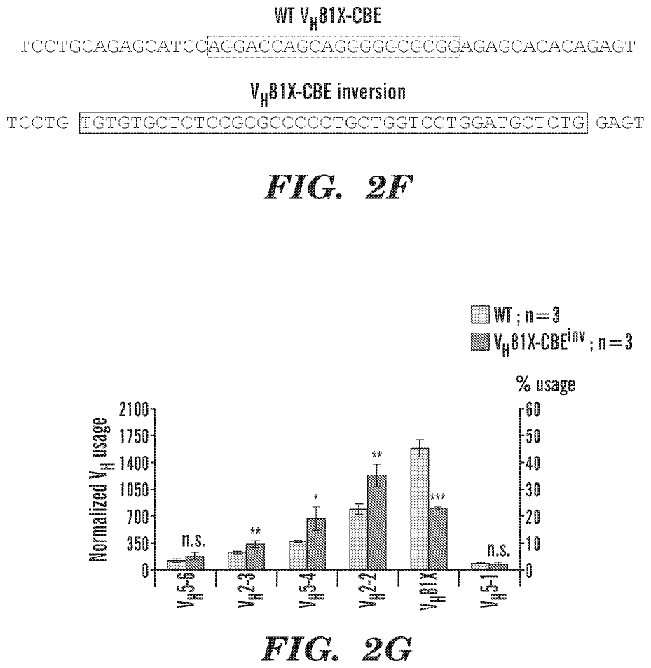

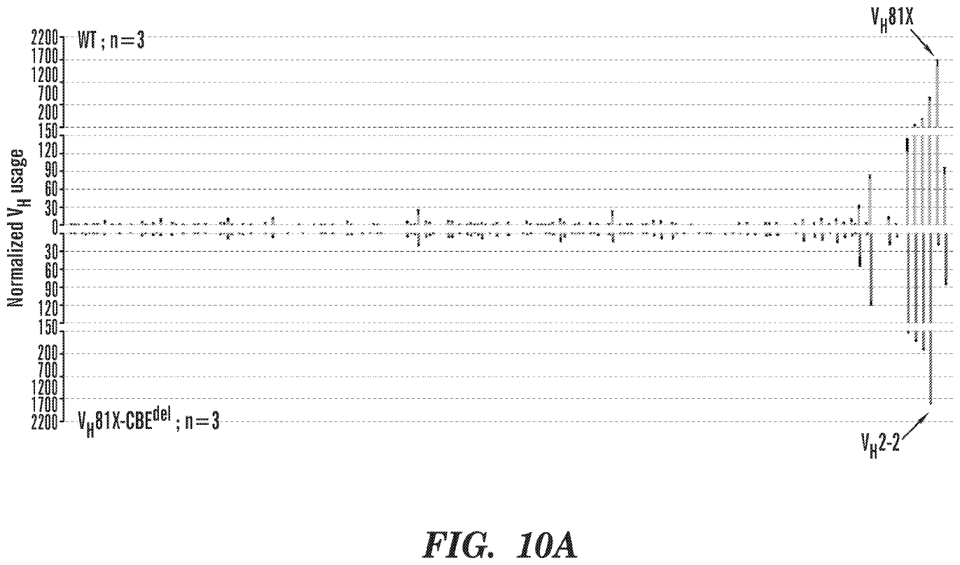

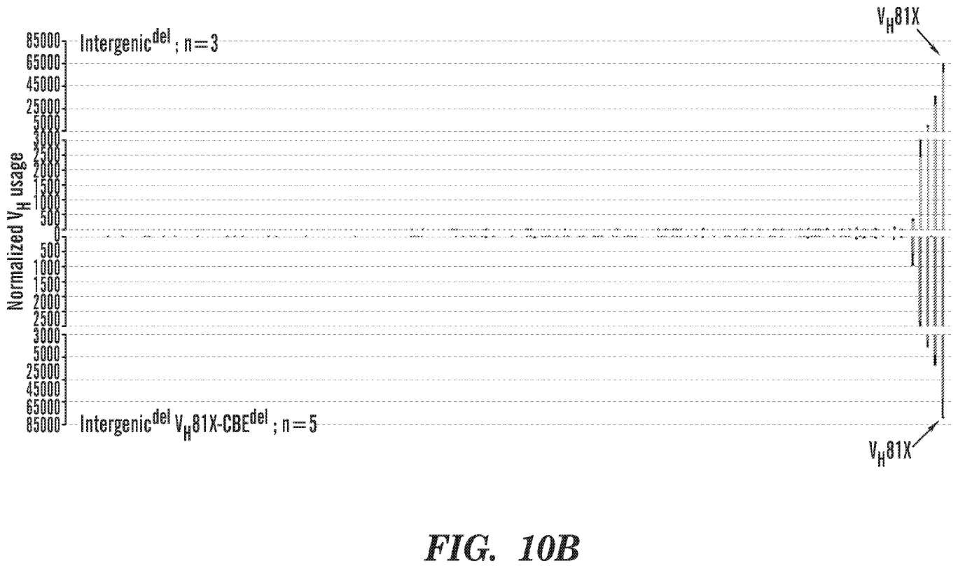

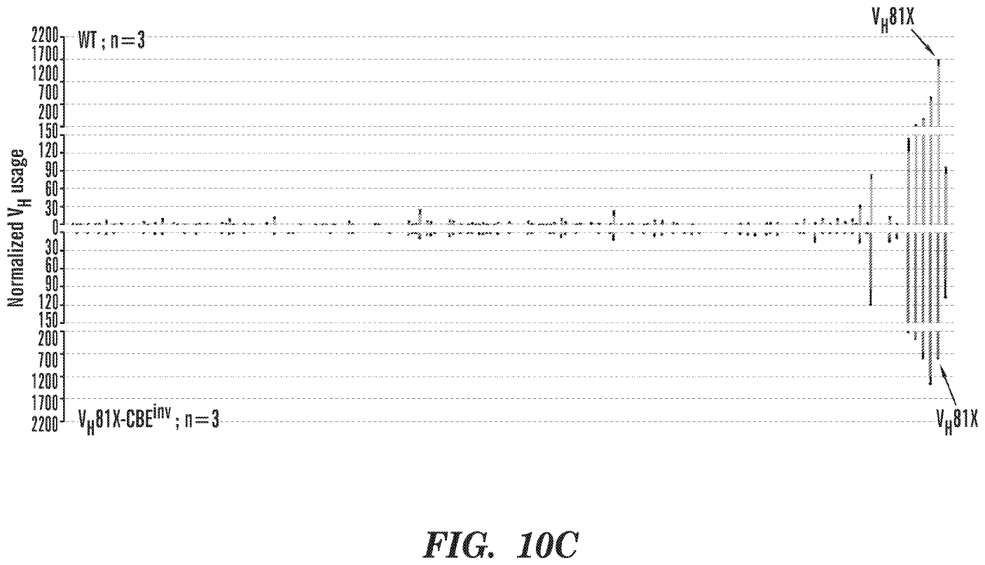

To establish a cell culture model to facilitate further analyses of VH81X-CBE function in V(D)J recombination, it was first tested whether this element is required for VH81X rearrangement in v-Abl transformed, Ep-Bcl2-expressing pro-B cells viably arrested in the G1 cell-cycle phase by treatment with STI-571 to induce RAG expression and V(D)J recombination (Bredemeyer et al., 2006). For this purpose, a v-Abl pro-B line was derived that harbors an inert non-productive rearrangement of a distal VHJ558 that deletes all proximal VHs and Ds on one allele and a DHFL16.1 to JH4 rearrangement that actively undergoes VH to DJH recombination on the other allele (FIG. 2A). Like an ATM-deficient DJH-rearranged v-Abl pro-B line (Hu et al., 2015), the DHFL16.1JH4 v-Abl pro-B line predominantly rearranges the most proximal VHs with only low level distal VH rearrangement due to lack of lgh locus contraction in v-Abl lines (FIG. 10A). Also employed was a Cas9/gRNA approach to generate a derivative of the DHFL16.1JH4 line in which the VH81X-CBE (referred to as “VH81X-CBEdel” mutation) on the DJH allele was deleted (FIG. 2B).

Three separate HTGTS-Rep-Seq libraries were analyzed from both parent and VH81X-CBEdel DHFL16.1JH4 v-Abl pro-B lines. These analyses revealed that VH81X is utilized in approximately 45% of VDJH rearrangements in the parent line, but in only about 0.5% of VDJH rearrangements in the VH81X-CBEdel line, representing a 100-fold decrease (FIGS. 2C and 10A; Table
1). Likewise, in VH81X-CBEdel DHFL16.1JH4 v-Abl cells, corresponding increases in utilization of the four VHs upstream of VH81X were observed with relative utilization patterns similar to those observed in VH81X-CBEscr/scr BM pro-B cells and no change in utilization of the downstream VH5-1 (FIG. 2C; Table 1). Based on these findings it was concluded that the various effects of VH81X-CBEdel mutation on utilization of VH81X and upstream neighboring proximal VHs are essentially identical in developing mouse pro-B cells and the DHFL16.1JH4 v-Abl pro-B cell line. Therefore, this v-Abl pro-B line was employed to further extend these studies and address mechanism. VH81X-CBE Mutation Does Not Impair VH RSS Functionality for V(D)J Recombination

Sequencing VH81X-CBE scrambled and deletion mutations in genomic DNA confirmed that both left the VH81X-RSS intact. Yet, the effect of VH81X-CBE mutations is nearly as profound and specific as expected for mutation of an RSS. To confirm that basic VH81X-RSS functions were intact subsequent to CBE deletion, a Cas9/gRNA approach was used to delete the approximately 101 kb sequence downstream of the VH81X-RSS in both DHFL16.1JH4 and VH81X-CBEdel DHFL16.1JH4 v-Abl cells, thereby positioning VH81X and its canonical RSS approximately 700 bp upstream of the DJHRC in both lines (FIG. 2D). This large intergenic deletion mutation (referred to as “Intergenicdel”), which removes IGCR1 and VH5-1, led to a 30-fold increase in overall VH to DJH joining levels in both the DHFL16.1JH4 and VH81X-CBEdel DHFL16.1JH4 v-Abl lines (Table 2). Comparative HTGTS-Rep-Seq analyses of multiple libraries from Intergenicdel and Intergenicdel VH81X-CBEdel DHFL16.1JH4 v-Abl lines demonstrated that 60% of the overall increase in VDJH junctions in both lines involved VH81X and that the remainder was contributed by proximal VHs just upstream (FIGS. 2E and 10B). Indeed, VH to DJH rearrangement levels and patterns in the parental and VH81X-CBEdel v-Abl lines harboring the large intergenic deletion were essentially indistinguishable (FIGS. 2E and 10B; Table 1). Thus, elimination of the VH81X-CBE does not alter ability of VH81X to undergo robust V(D)J recombination when VH81X is positioned near the DJHRC, indicating that the VH81X-CBE V(D)J recombination function is manifested at a different level than RSS-dependent RAG cleavage.

The VH81X-CBE Mediates Robust VH81X Rearrangement When Inverted

Several studies indicated that CBE orientation is critical for its function as a loop domain anchor (Rao et al., 2014; Sanborn et al., 2015), as well as for mediating enhancer-promoter interactions (Guo et al., 2015; de Wit et al., 2015) and regulating alternative splicing (Ruiz-Velasco et al., 2017). Convergent VH-CBE orientation with respect to IGCR1-CBE1 and the 3′CBEs suggested that such organization may be important for V(D)J recombination regulation (Guo et al., 2011; Lin et al., 2015; Benner et al., 2015; Aiden and Casellas, 2015; Proudhon et al., 2015). To test this notion, a Cas9/gRNA approach was used to invert a 40-bp sequence encompassing VH81X-CBE in the DHFL16.1JH4 v-Abl line to generate “VH81X-CBEinv” lines (FIG. 2F). Comparative HTGTS-Rep-Seq analyses of multiple libraries from parent and VH81X-CBEinv lines demonstrated that inversion of the VH81X-CBE resulted in only an approximately 2-fold decrease in VH81X utilization (FIGS. 2G and 10C; Table 1), as compared to the 100-fold reduction observed upon VH81X-CBE deletion (FIG. 2C; Table 1). Thus, the VH81X-CBE in inverted orientation supports reduced, but still robust, VH81X utilization.

VH81X-CBE Promotes Interaction with the DJHnRC

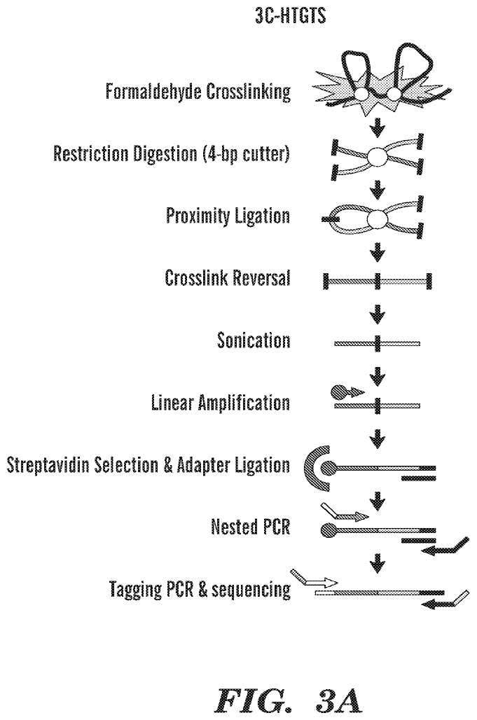

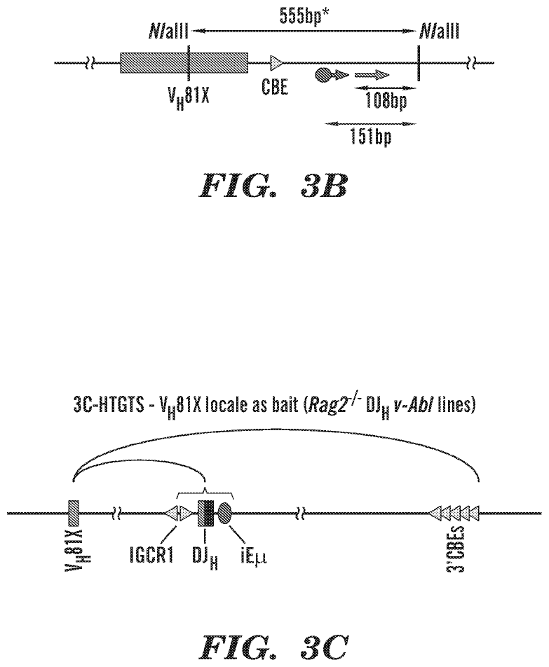

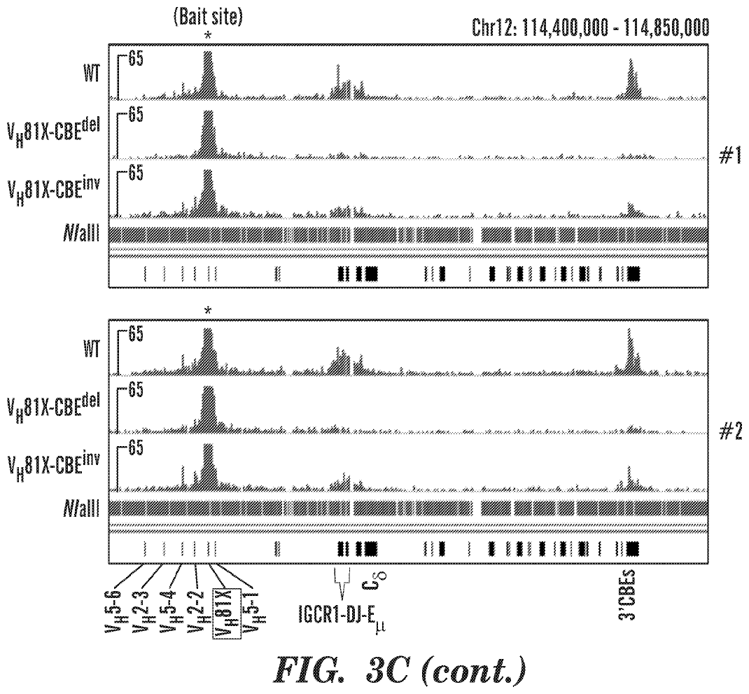

To examine VH81X-CBE interactions with other Igh regions, an HTGTS-based methodology that provides high-resolution and reproducible interaction profiles of a bait locale of interest with unknown (prey) interacting sequences across Igh was developed (FIG. 3A). For this method, termed 3C-HTGTS, a 3C library (Dekker et al., 2002) was prepared with a 4-bp cutting restriction endonuclease and, after the sonication step, employment of linear amplification-mediated-HTGTS (Frock et al., 2015; Hu et al., 2016) to complete and analyze the libraries (See STAR Methods). For the present purposes, 3C-HTGTS substitutes well for prior 4C-related approaches (Denker and de Laat, 2016). In this regard, use of linear amplification to enrich for ligated products allows 3C-HTGTS to generate highly sensitive and specific interaction profiles for widely separated bait and prey sequences (FIG. 3C). As all pro-B line Igh chromatin interaction experiments must be done in the context of RAG-deficiency to avoid confounding effects of ongoing V(D)J recombination, a Cas9/gRNA approach was used to derive RAG2-deficient derivatives of the various v-Abl lines.

To identify interaction partners of VH81X, 3C-HTGTS was performed on RAG2-deficient derivatives of control, VH81X-CBEdel, and VH81X-CBEinv DHFL16.1JH4 v-Abl lines using VH81X as bait (FIG. 3B). In control RAG2-deficient DHFL16.1JH4 v-Abl cells, VH81X reproducibly interacts specifically with a region 100 kb downstream that spans IGCR1 and the closely linked (3 kb downstream) DJHnRC locale, as well as with a region 300 kb downstream containing the 3′ Igh CBEs (FIG. 3C). Both of these interactions are dependent on the VH81X-CBE, as they are essentially absent in VH81X-CBEdel RAG2-deficient DHFL16.1JH4 v-Abl cells (FIG. 3C). However, 3C-HTGTS analyses of the VH81X-CBEinv RAG2-deficient DHFL16.1JH4 v-Abl cells revealed significant VH81X interactions with IGCR1/DJHnRC and 3′CBEs, albeit at moderately reduced levels compared to those of RAG2-deficient DHFL16.1JH4 control v-Abl cells (FIG. 3C). Thus, levels of VH81X interactions with IGCR1/DJHnRC locale and 3′CBEs in VH81X-CBE inversion and deletion mutants reflect VH81X utilization in these mutants relative to the parental DHFL16.1JH4 v-Abl lines, implying a potential mechanistic relationship between these interactions and VH81X utilization.

### V(D)J Recombination of VH2-2 is Critically Dependent on its its Flanking CBE

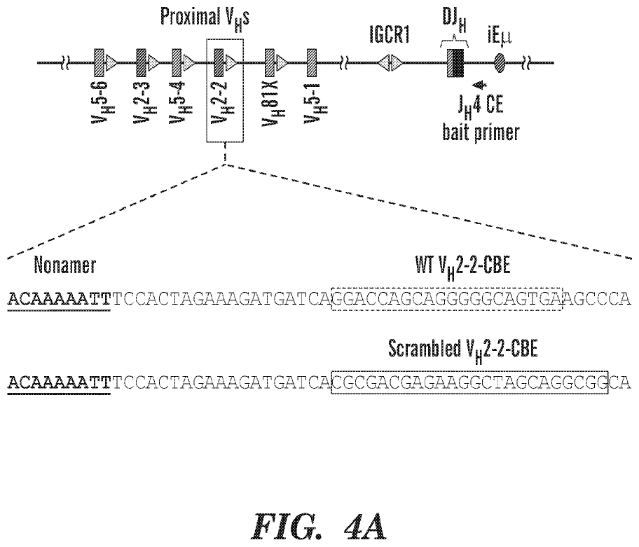

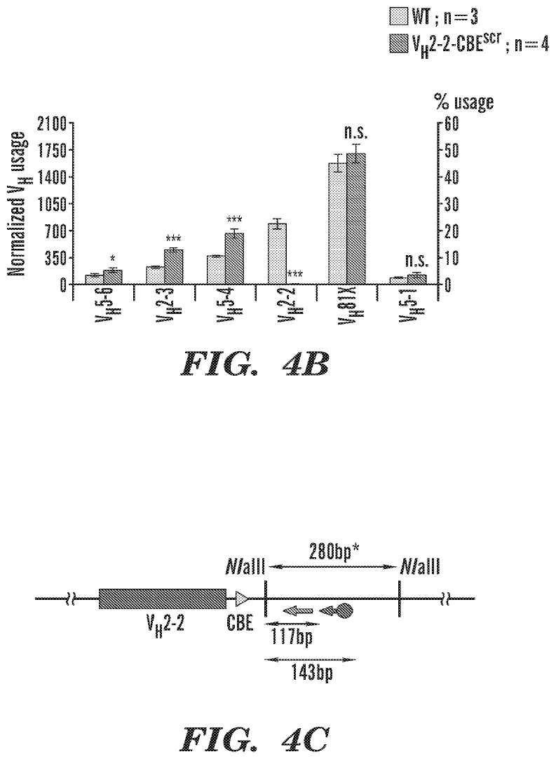

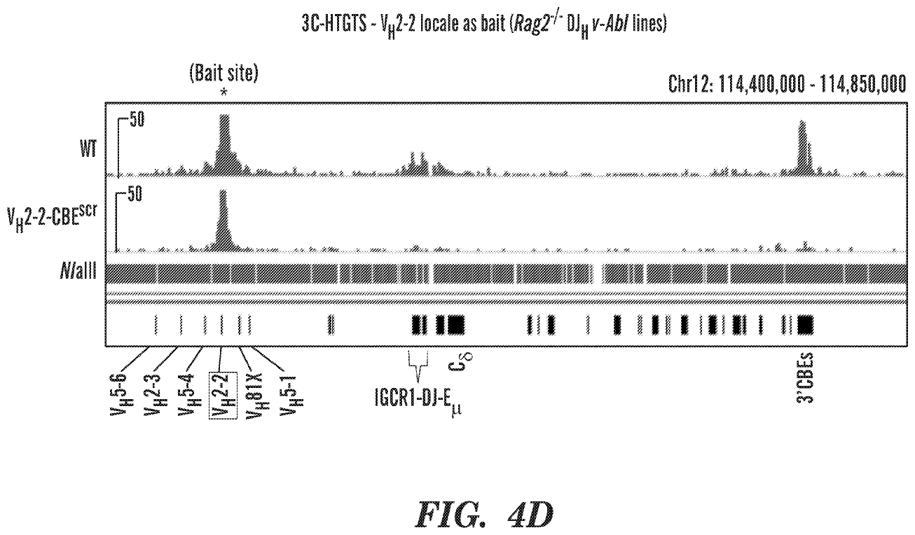

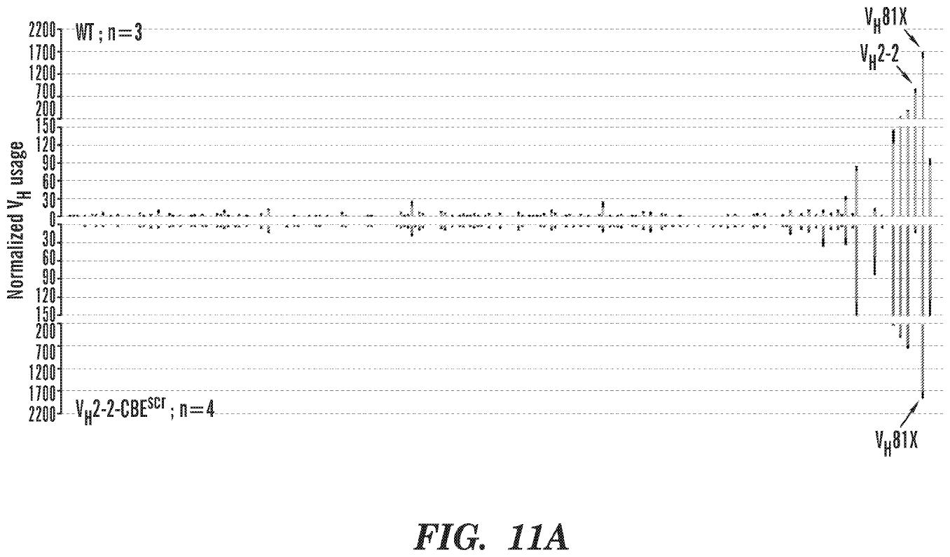

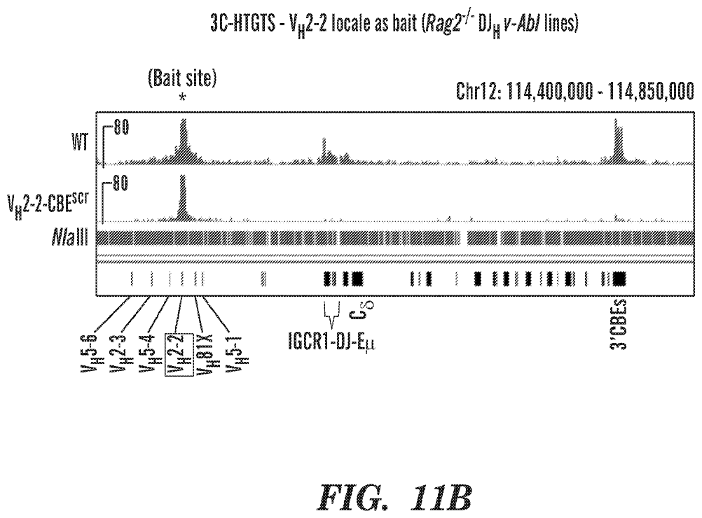

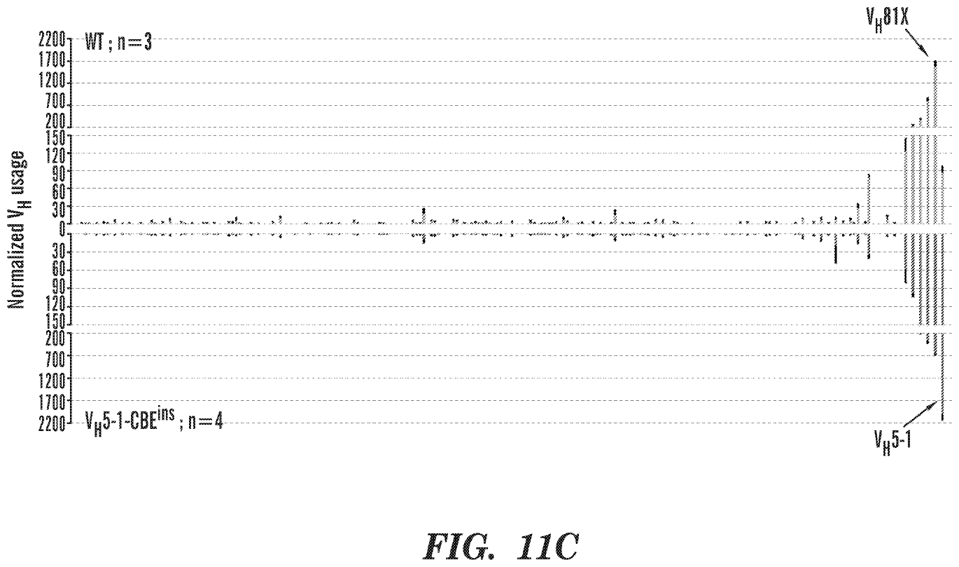

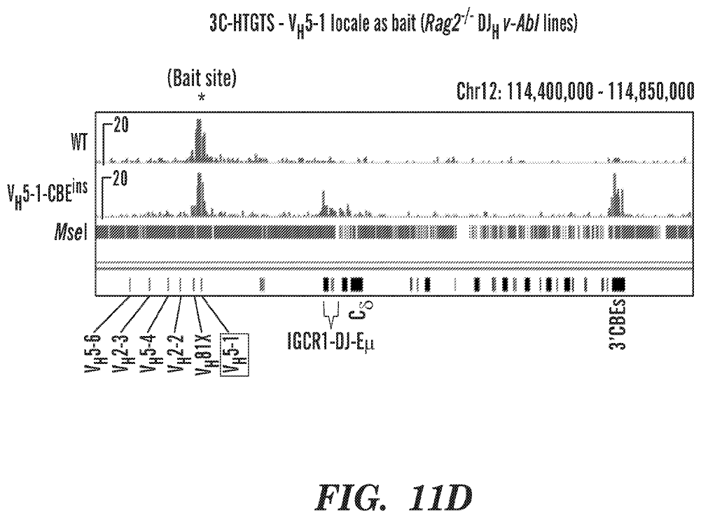

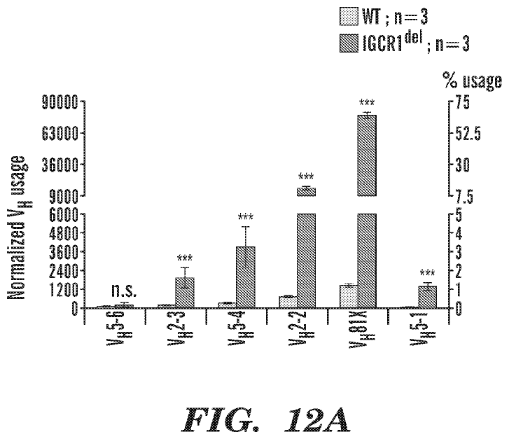

To test the function of an additional VH-associated CBE, “VH2-2-CBEscr” DHFL16.1JH4 v-Abl lines weew generated in which the CBE just downstream of VH2-2 was replaced with a scrambled sequence that does not bind CTCF (FIG. 4A). Comparative analyses of multiple HTGTS-Rep-Seq libraries from the parental versus VH2-2-CBEscr mutant DHFL16.1JH4 lines demonstrated that the VH2-2-CBE-scrambled mutation reduced VH2-2 utilization nearly 100-fold in the VH2-2-CBEscr line (FIGS. 4B and 11A; Table 1). In addition, the VH2-2-CBEscr mutation led to increased utilization of the three VHs immediately upstream of VH2-2, but had no effect on utilization of the downstream VH81X and the VH5-1 pseudo-VH (FIG. 4B). 3C-HTGTS assays performed on RAG2-deficient parental and VH2-2-CBEscr RAG2-deficient DHFL16.1JH4 v-Abl lines showed that VH2-2, like VH81X, significantly interacts with the IGCR1/DJHnRC locale and the 3′CBEs in a VH2-2-CBE-dependent manner (FIGS. 4C, 4D and 11B). Thus, the various effects of VH2-2-CBEscr mutation on VH2-2 utilization, utilization of neighboring VHs, and long-range interactions with downstream Igh IGCR1/DJHnRC locale corresponds well with those associated with deletion of the VH81X-CBE.

CBE-Dependent VH81X Dominance without IGCR1 Implicates RAG Chromatin Tracking

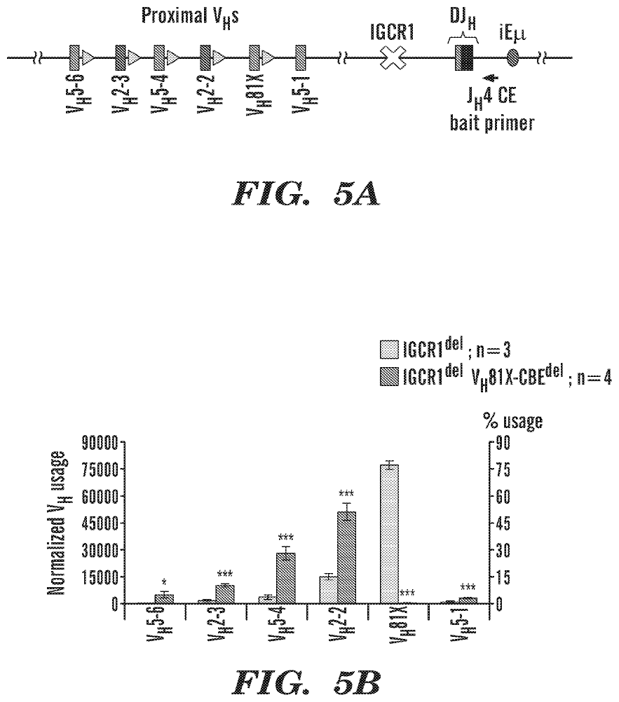

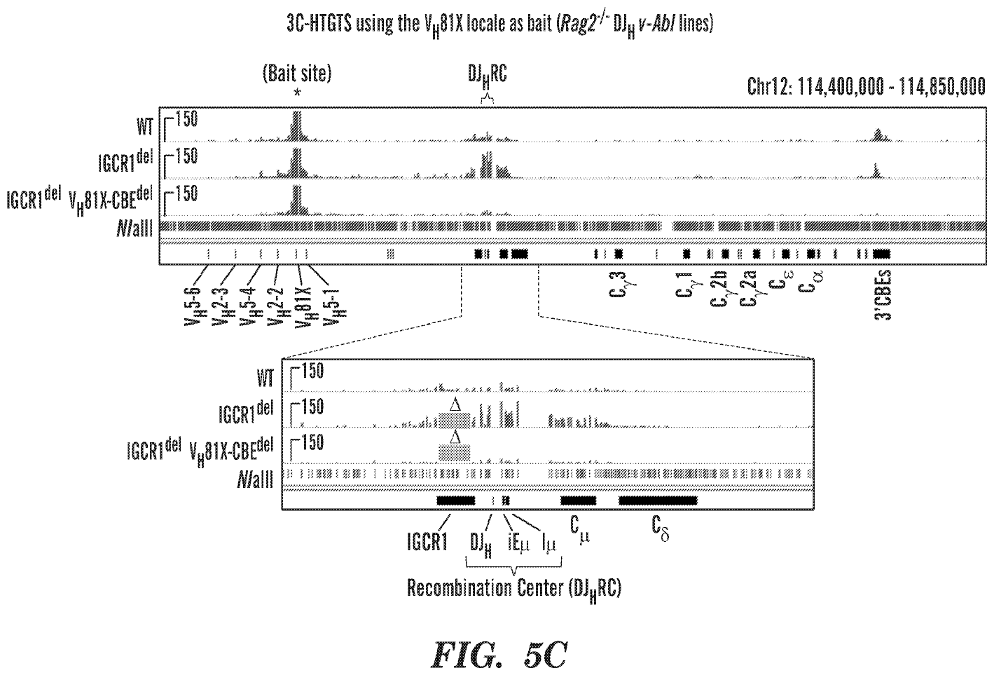

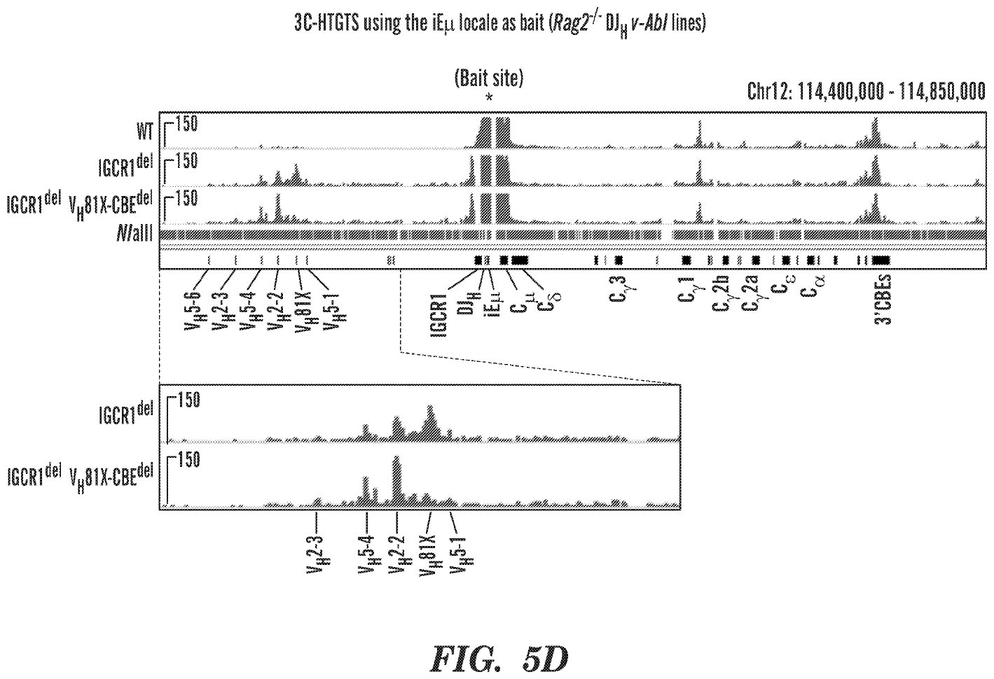

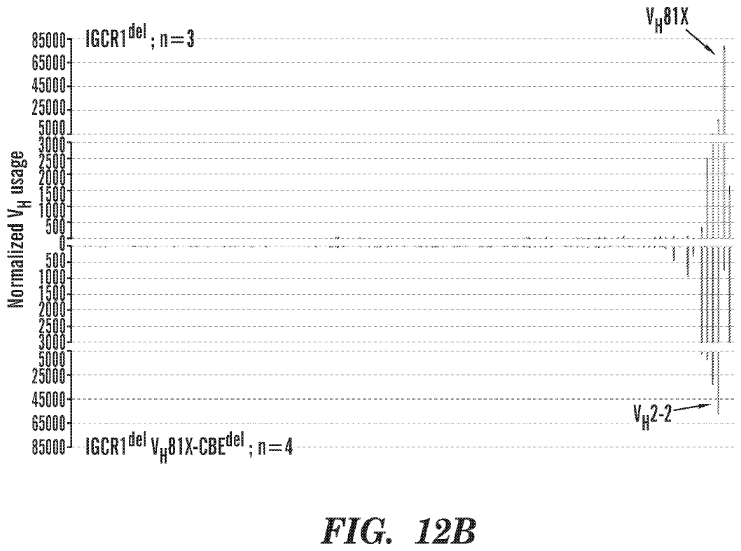

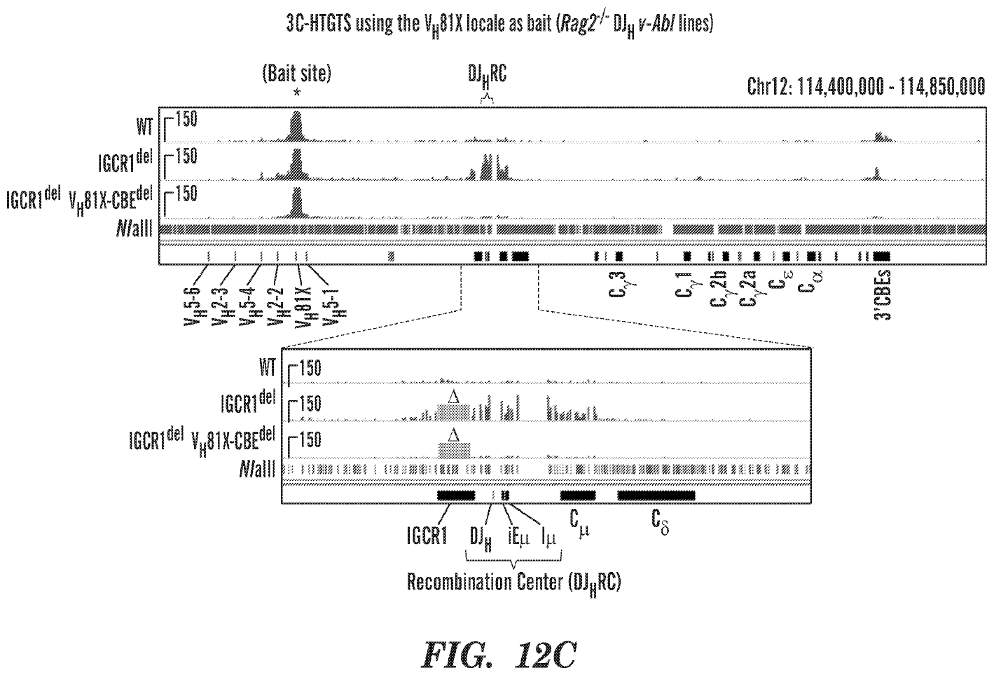

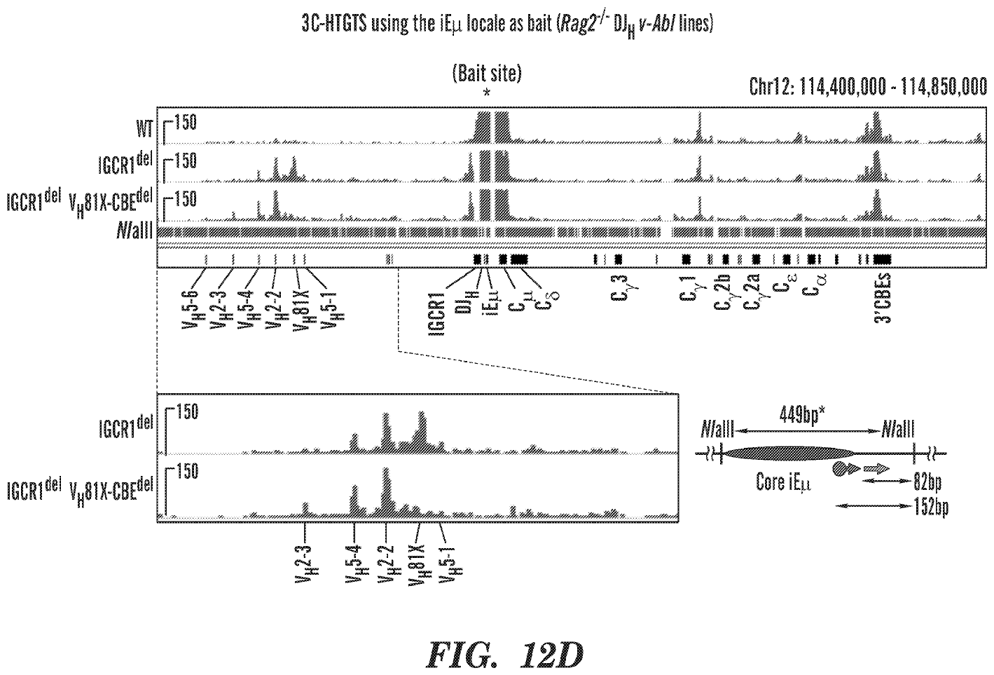

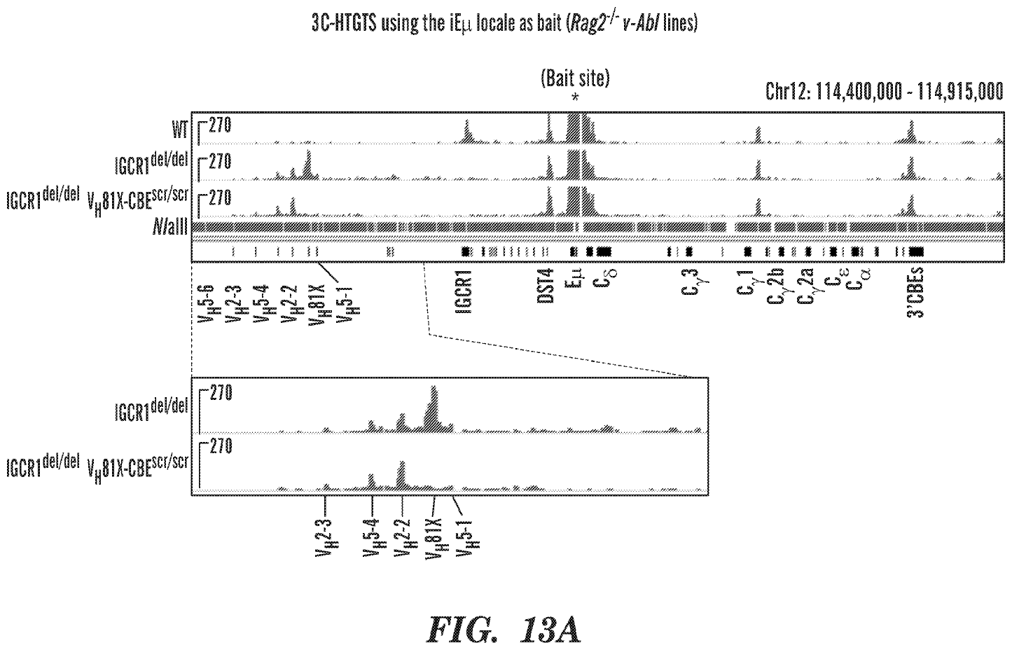

IGCR1 deletion results in tremendous over-utilization of proximal VHs, most dramatically VH81X, in association with RAG linear exploration of sequences upstream of IGCR1 via some form of tracking (Hu et al., 2015). To test whether the VH81X-CBE contributes to the immense over-utilization of VH81X in the context of IGCR1 deletion and RAG tracking, IGCR1-deleted (“IGCR1del”) DHFL16.1JH4 v-Abl cells were generated with or without the VH81X-CBEdel mutation (FIG. 5A). As expected, IGCR1 deletion led to a 30-fold increase in overall VH to DJH joining levels as compared to those of the DHFL16.1JH4 parent line, involving most predominantly VH81X and to a lesser extent proximal upstream VHs and the downstream VH5-1 (Tables 1 and 2; FIG. 12A). Comparative analyses of multiple HTGTS-Rep-Seq libraries from IGCR1del versus IGCR1del VH81X-CBEdel DHFL16.1JH4 lines revealed more than a 100-fold decrease in VH81X utilization in the IGCR1del VH81X-CBEdel line versus the IGCR1del line (FIGS. 5B and 111B; Table 1). Once again, this dramatic decrease in VH81X utilization was accompanied by increased utilization of the four VHs immediately upstream of VH81X (FIG. 5B; Table 1).

To identify VH81X-CBE interaction partners in the context of IGCR1-deficiency, 3C-HTGTS was performed using VH81X bait on RAG2-deficient DHFL16.1JH4 v-Abl cells that also harbored either IGCR1del or IGCR1del VH81X-CBEdel mutations (FIG. 5C). As described above (FIG. 3C), VH81X has significant VH81X-CBE-dependent interactions with the 1GCR1/DJHnRC locale and the 3′CBEs in RAG2-deficient DHFL16.1JH4 v-Abl cells. However, in RAG2-deficient IGCR1del lines, VH81X interaction with the DJHnRC locale, which we can now pinpoint in the absence of IGCR1, occurs at far higher levels than its interaction with the 1GCR1/DJHnRC locale in RAG2-deficient DHFL16.1JH4 v-Abl parent line, even though interactions with the 3′CBEs remain the same or are slightly decreased (FIGS. 5C and 12C; top and bottom zoomed-in panels). Strikingly, in RAG2-deficient IGCR1del VH81X-CBEdel lines, VH81X interactions with the DJHnRC and 3′CBEs were essentially eliminated (FIGS. 5C and 12C; top and bottom zoomed-in panels).

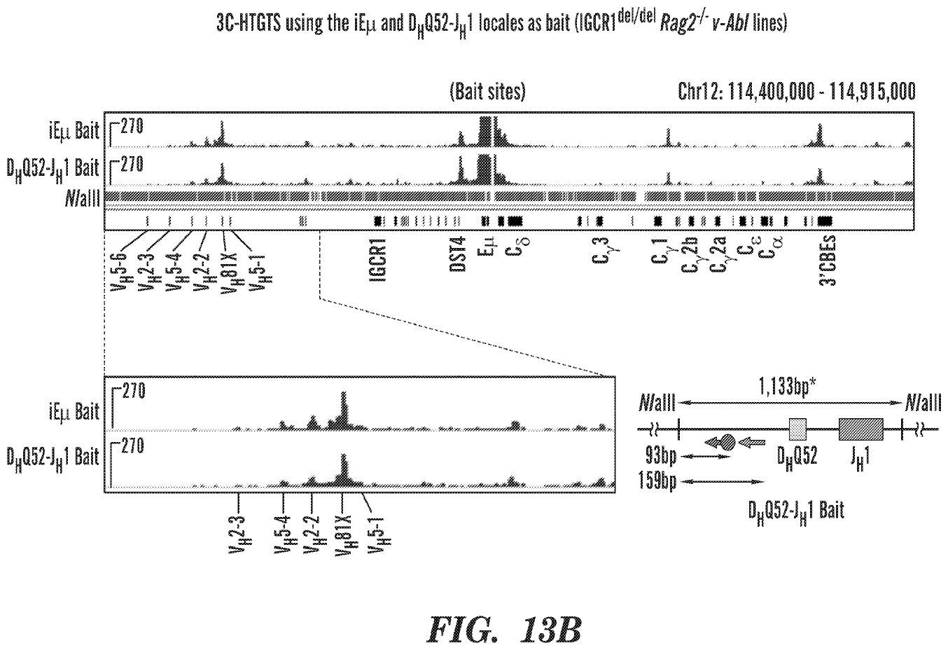

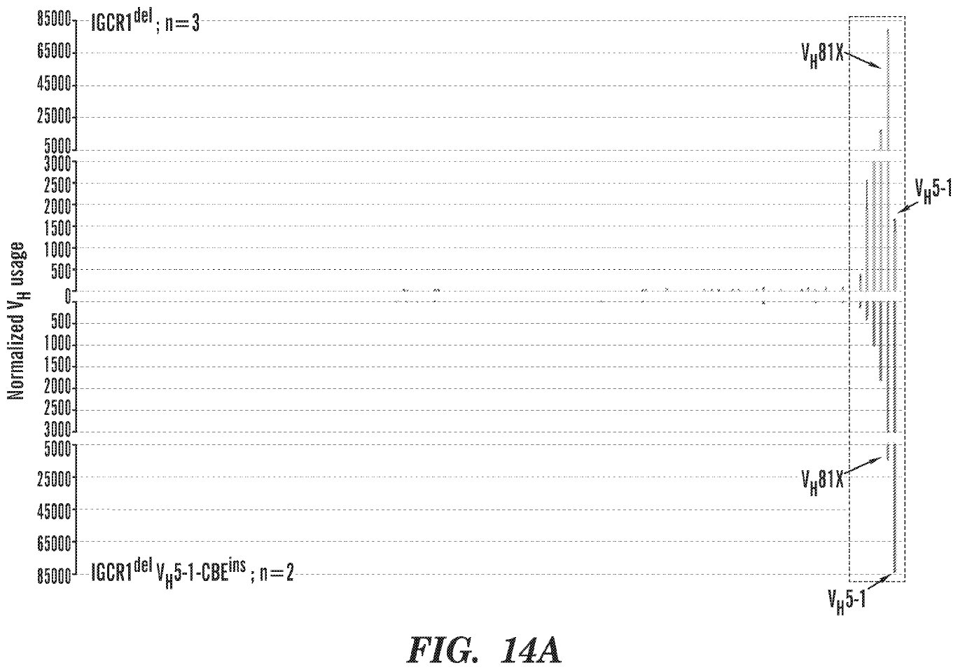

We also used iEμ within the DJHnRC as bait to examine interactions with other Igh sequences in this same set of RAG2-deficient control, IGCR1del, and IGCR1del VH81X-CBEdel DHFL16.1JH4 v-Abl lines. In all three genotypes, iEμ interacted with the 3′CBEs and with a region between Cγ1 and Cγ2b (Medvedovic et al., 2013). In the RAG2-deficient DHFL16.1JH4 control line, iEμ has barely detectable interaction with proximal VHs (FIGS. 5D and 12D; top panel). However, in RAG2-deficient IGCR1del lines, iEμ robustly interacts with VH81X and, at decreasing levels, with the upstream VH2-2 and VH5-4. In the RAG2-deficient IGCR1del VH81X-CBEdel lines, interactions between iEμ and VH81X decreased dramatically while interactions with the immediately upstream VH2-2 increased (FIGS. 5D and 12D; top and bottom zoomed-in panels). the iEμ as well as another DHQ52-JH1 locale bait, were also employed as a distinct nRC bait for 3C-HTGTS assays in RAG2-deficient control and IGCR1del/del v-Abl lines with an unrearranged Igh locus and found essentially identical interaction profiles (FIG. 13A-13B). Together, these 3C-HTGTS studies indicate that the impact of IGCR1 deletion on dramatically increased CBE-dependent utilization of proximal VHs in RAG2-sufficient WT and mutant lines directly correlates with their interaction with the DJHnRC in their RAG2-deficient counterparts.

Restoration of a Vestigial CBE Converts VH5-1 into the Most Highly Rearranging VH

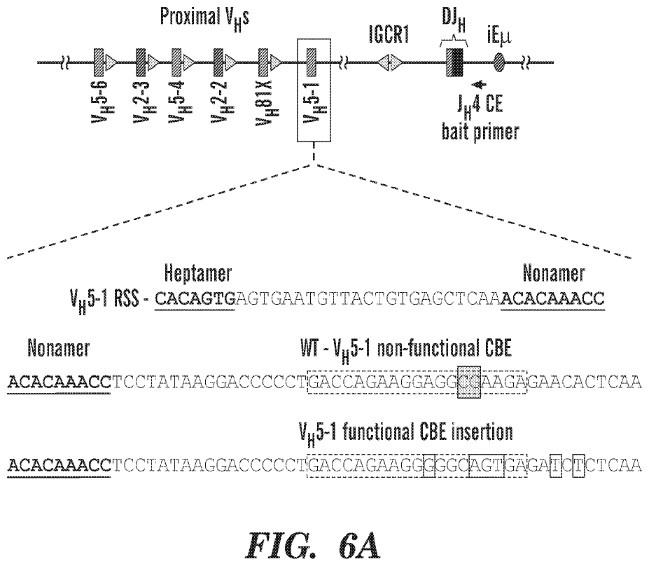

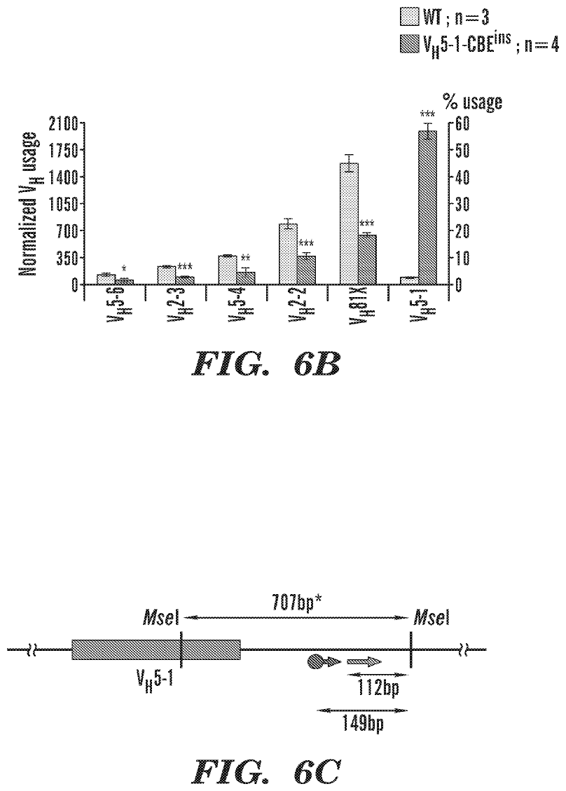

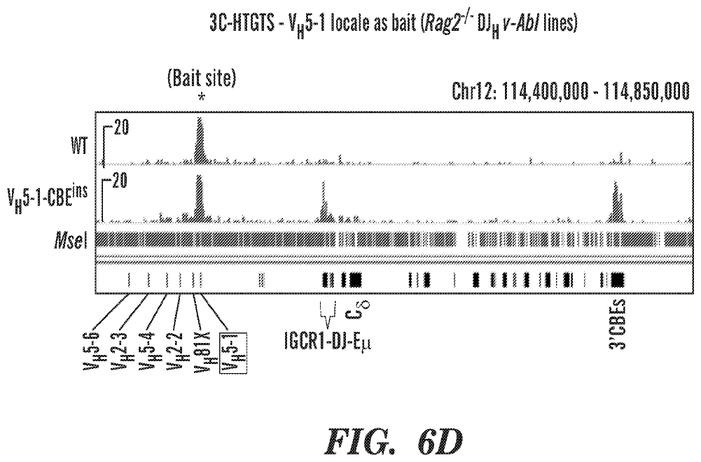

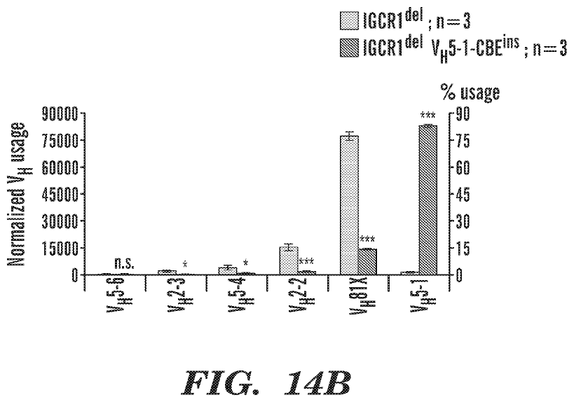

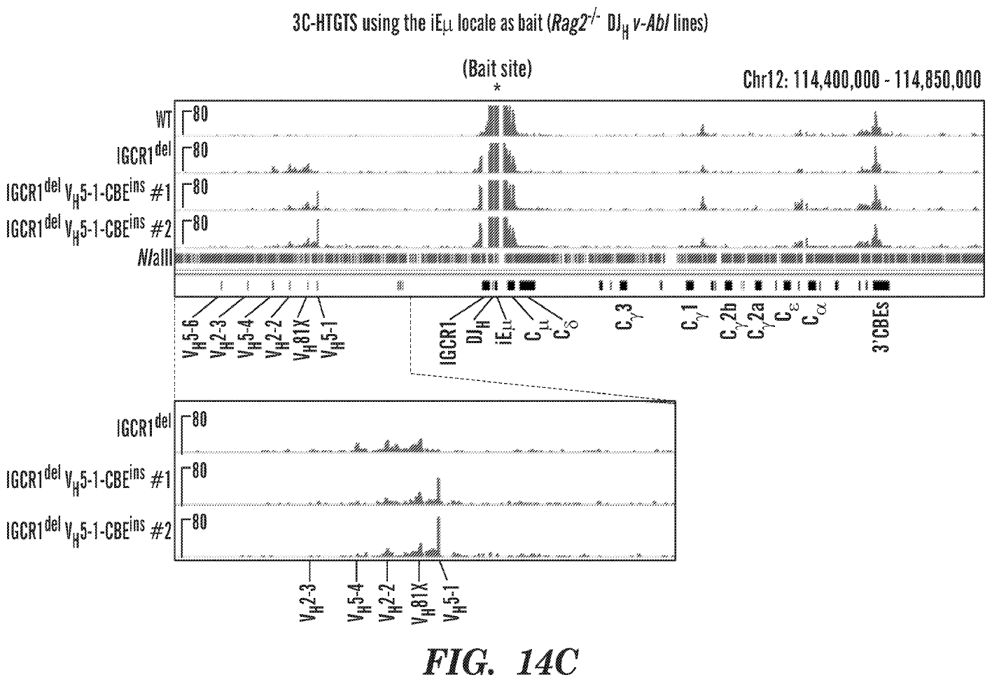

Mutation of the VH81X or VH2-2 CBEs remarkably reduce ability of these VHs to be utilized for V(D)J recombination, despite retention of their normal RSSs. In this regard, the most D-proximal VH5-1 has a canonical RSS (FIG. 6A), but is infrequently rearranged in WT pro-B cells or v-Abl pro-B lines (Hu et al., 2015; FIGS. 1C and 2C; Table 1). By employing a JASPAR sequence-based prediction, it was found that VH5-1 also is flanked downstream of its RSS by a CBE-related sequence (FIG. 6A), the site of which is CpG methylated and does not bind CTCF in pro-B cells (Benner et al., 2015). To test if lack of a functional CBE causes infrequent VH5-1 utilization, DHFL16.1JH4 v-Abl lines (referred to as “VH5-1-CBEins”) were generated in which 4 bps within this putative vestigial CBE were mutated to eliminate the CpG island and generate a consensus CTCF-binding element (FIG. 6A). Comparative analyses of multiple HTGTS-Rep-Seq libraries from the parental and VH5-1-CBEins DHFL16.1JH4 lines demonstrated that generation of VH5-1-CBE resulted in over a 20-fold increase in VH5-1 utilization, converting it into the most highly utilized VH (FIGS. 6B and S4C; Table 1). Notably, this gain of function VH5-1-CBEins mutation also decreased utilization of the immediately upstream VH81X and the next four upstream VHs, with their reduced utilization levels corresponding linearly with increasing distance upstream (FIG. 6B). Strikingly, 3C-HTGTS studies on RAG2-deficient VH5-1-CBEins lines demonstrated that restoration of the VH5-1-CBE also promoted significant gain of function interactions of VH5-1 with the IGCR1/DJHnRC locale and 3′CBEs (FIGS. 6C, 6D and 1 ID), further supporting direct links between VH recombination potential and these interactions. Finally, IGCR1 was deleted in the VH5-1-CBEins line, which led to an approximately 60-fold increase in VH5-1 utilization with dramatically decreased utilization of VH81X and other upstream proximal VHs (FIGS. 14A and 14B). Likewise, in 3C-HTGTS experiments VH5-1 gained dramatically increased interactions with the DJHnRC as viewed from an iEμ bait (FIG. 14C).

### Discussion

### Proximal VH-CBEs Enhance V(D)J Recombination Potential of Associated VHs

Described herein is the major role of VH-associated CBEs in V(D)J recombination. Thus, V(D)J recombination potential of VH81X is dramatically enhanced in both primary pro-B cells in mice and in v-Abl pro-B lines by its associated CBE. Likewise, V(D)J recombination potential of the upstream VH2-2 is similarly enhanced by its associated CBE. Decades ago, we hypothesized one dimensional “recombinase scanning” as a possible mechanism for preferential proximal VH utilization, but noted that there must be an additional determinant based on low level VH5-1 pseudo-VH utilization despite its most proximal location downstream of VH81X and consensus RSS (Yancopoulos et al., 1984). Described herein is this additional determinant as a CBE by converting the “vestigial” CBE downstream of VH5-1 into a functional CBE and, thereby, rendering it the most frequently rearranged VH. However, the VH81X-CBE was not required for robust VH81X rearrangement when it was placed linearly adjacent to the DJHRC, indicating VH-CBE function is distinct from that of RSSs. To further assess the mechanism by which proximal VH-CBEs enhance V(D)J recombination potential, a highly sensitive 3C-HTGTS chromatin interaction method was developed. Effects of various tested loss and gain of function CBE mutations on V(D)J recombination potential of the 3 proximal VHs were mirrored by effects on their interactions with the DJHnRC. This relationship was most striking in the context of IGCR1 deletion, which leads to both dramatically increased VH81X utilization and dramatically increased VH81X interaction with the DJHnRC, with both increases being dependent on the VH81X-CBE. Thus, proximal VH-CBEs increase V(D)J recombination potential by increasing the frequency with which their associated VHs interact with the DJHRC.

### VH-CBEs Mediate RSS Accessibility During RAG Chromatin Scanning

RAG tracking in the absence of IGCR1 proceeds upstream to the most proximal VHs, resulting in their increased rearrangement to DJH intermediates (Hu et al., 2015). This dominant increase in VH81X rearrangement during tracking in the absence of IGCR1 is VH81X-CBE-dependent and associated with CBE-mediated DJHRC interactions. The imprint of linear tracking on proximal VH utilization in the absence of IGCR1 goes beyond VH81X. Thus, in v-Abl pro-B lines, where tracking effects are more pronounced in the absence of locus contraction, the three VHs just upstream of VH81X also show markedly increased utilization with relative utilization decreasing with upstream distance. Likewise, while VH81X utilization plummets in VH81X-CBEdel v-Abl cells lacking IGCR1, utilization of the upstream VH2-2 becomes dominant and that of the three upstream VHs again increases with levels inversely related to upstream distance. Also consistent with linear tracking, utilization of the most downstream CBE-less VH5-1 with a restored CBE increases substantially in the absence of IGCR1 becoming dominant even over VH81X. Relative VH utilization patterns during RAG upstream tracking in the absence of IGCR1 correlate well with proximal VH interactions with the DJHnRC. Together, these findings indicate that RAG scans chromatin, rather than DNA per se, allowing this process to be better described as linear RAG chromatin scanning; and they further indicate that proximal VH-CBEs promote over-utilization of associated VHs via a chromatin accessibility-enhancing function. The mechanism of this accessibility function likely involves CBE-mediated prolonged interaction of the VH with the DJHRC. It is described herein that long-range interactions critical to RAG chromatin scanning do not require a functional RAG complex. Thus, RAG bound to the DJHRC may harness a more general cellular mechanism operating within the Igh locus, such as cohesin-mediated chromatin loop extrusion, to scan distal sequences.

RAG Chromatin Scanning Shares Features with Chromatin Loop Extrusion

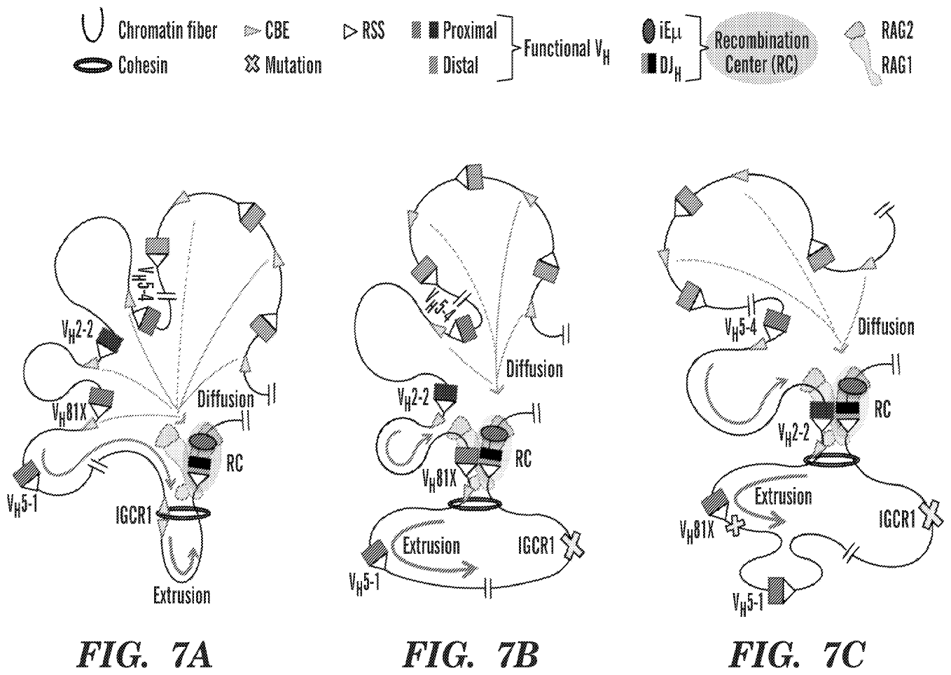

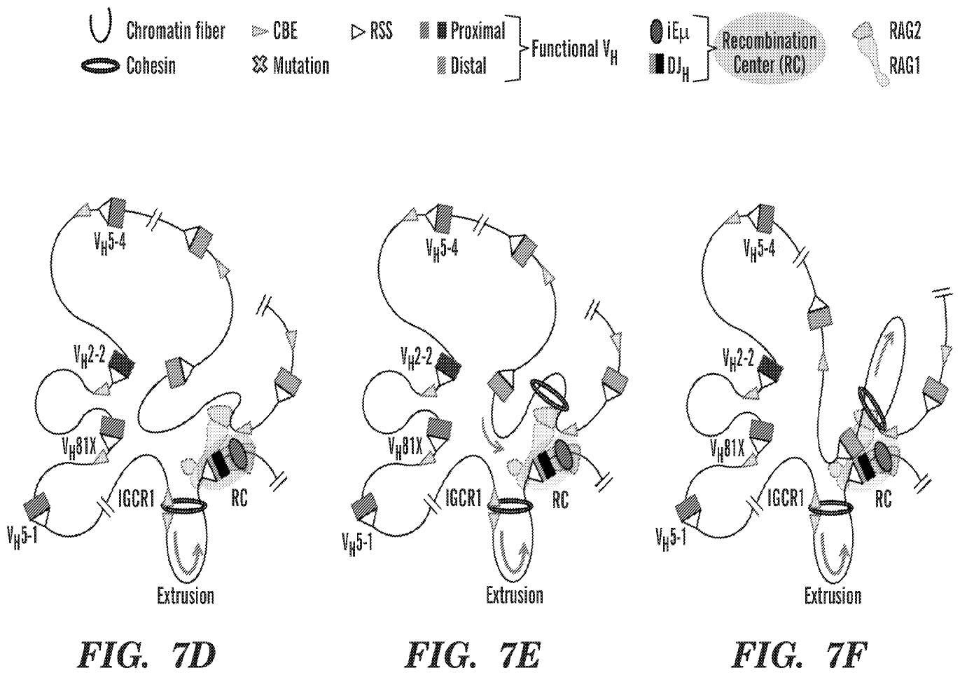

Inserting RSS pairs to generate ectopic “RCs” in various random genomic sites revealed orientation-specific linear RAG chromatin scanning within chromosome loop domains bounded by convergent CBE anchors, suggesting cohesin involvement (Hu et al., 2015). Features of RAG scanning overlap with those of cohesin-mediated loop extrusion (Dixon et al., 2016; Dekker and Mirny, 2016). Cohesin rings extrude chromatin loops that become progressively larger, bringing distal chromosomal regions into physical proximity in a linear fashion and having the potential to increase contact frequencies between loop anchors and sequences across extrusion domains (Fudenberg et al., 2016; Rao et al., 2017; Sanborn et al., 2015; Schwarzer et al., 2017). In this regard, CBEs bound by CTCF act as strong loop anchors and impede extrusion (Nichols and Corces, 2015; Fudenberg et al., 2016; Nora et al., 2017). Overlaps between loop extrusion and RAG scanning suggest that scanning may be driven by chromatin extrusion past a RAG-containing “RC anchor” (FIG. 7). While convergent CBE anchors substantially block extrusion, other chromatin structures, such as enhancers, can impede extrusion (Dekker and Mirny, 2016). Thus, based on interactions in pro-B cells (Guo et al., 2011; Medvedovic et al., 2013; this study), IGCR1 and the JHRC may act as upstream and downstream barriers to loop extrusion-mediated RAG scanning during D to JH recombination. Deletion of IGCR1 would eliminate the upstream barrier and extend extrusion into proximal VHs, allowing VH CTCF/cohesin-bound CBE interactions with the downstream RC extrusion anchor that increase accessibility of associated VHs. While VH-CBEs increase RC interaction frequencies, they do not create absolute boundaries, as RAG scanning can extend past them at decreased levels to immediately upstream VHs. In contrast to certain CBE-mediated looping and regulatory processes (Sanborn et al., 2015; Guo et al., 2015; de Wit et al., 2015), VH81X-CBE function during RAG scanning is moderately enhanced by, but not strictly dependent on, convergent orientation, likely due to stronger interactions in convergent orientation. Finally, proximal VH-CBEs, DJHRC and 3′CBEs all interact indicating 3′CBEs contribute to VH-DJHRC interactions. Thus it is contemplated herein that deleting all 3′CBEs may influence Igh V(D)J recombination more than deleting a subset (Volpi et al., 2012).

### Contribution of RAG Scanning to Proximal VH Usage in the Presence of IGCR1

After Igh locus contraction brings distal VHs into closer proximity of the DJHRC, they become directly associated with the RC via subsequent diffusion-related mechanisms (Lucas et al., 2014). Notably, however, utilization of the very most proximal VHs does not require locus contraction (Fuxa et al., 2004). In this regard, primary locus-contracted pro-B cells utilize VH81X and VH2-2 more frequently than more distal VHs. Likewise, in VH81X-CBE mutant primary pro-B cells utilization of the immediately upstream VH2-2 increases dramatically with utilization of the next two upstream VHs increasing to levels higher than those of more distal VHs. In v-Abl pro-B cells, which lack Igh contraction but have intact IGCR1, over-utilization of VH81X and the four immediately upstream VHs have a distance-dependent utilization pattern reminiscent of that when IGCR1 is inactivated. Likewise, deletion of the VH2-2-CBE increases relative utilization of upstream VHs, again with the same distance-related pattern, but has no effect on downstream VH81X utilization. Finally, ectopic introduction of an immediately downstream CBE renders proximal VH5-1 the most highly utilized VH, while, correspondingly, greatly dampening utilization of upstream VHs. Together, these findings indicate that the relatively high recombination potential of very most proximal functional VHs, even in normal, locus contracted pro-B cells, results from low level RAG chromatin scanning from the DJHRC into the proximal VH domain in the presence of IGCR1 CBEs. Beyond these proximal VHs, RAG linear scanning upstream from the DJHRC appears to have little, if any, impact, even in the absence of IGCR1; likely because dominant utilization of proximal VHs first encountered obviates most RAG scanning upstream.

### Potential Roles of CBEs and RAG Scanning in Distal VH Recombination

Nearly all functional mouse VHs have CBEs directly adjacent or within several kb (FIG. 8A-8E). In this regard, more distal VH-CBEs likely have V(D)J recombination functions related to those elucidated herein for CBEs of the very most proximal VHs. The VH portion of Igh comprises proximal, middle, J558 and distal J558/3609 VH regions with different chromatin and transcriptional properties (Choi et al., 2013; Bolland et al., 2016; FIG. 8A). The proximal and middle regions largely have repressive as opposed to active chromatin marks; and VHs within them, including VH81X, show little or no germline transcription. Correspondingly, the majority of proximal/middle VHs, in addition to the few accessible to RAG linear scanning, have CBEs adjacent to their RSSs that may stabilize diffusion-mediated interactions with the DJHRC to promote accessibility (FIGS. 8B, 8C and 7A). Notably, the J558 and, particularly, the distal J558/3609 regions have accessible chromatin marks and regions of transcription. In contrast to proximal VHs, few distal VHs are directly associated with a CBE, but most have CBEs within 10 kb and often much closer (FIGS. 8D and 8E). Such CBEs in distal domains still may enhance diffusion-mediated interactions with the DJHRC directly or in association with other interacting sequences such as IGCR1 or the 3′CBEs. Interactions with CBEs not directly associated with VHs also could provide anchors for loop extrusion of the locally accessible distal VHs past the RC (FIGS. 7D-7F). Thereby, distal VHs may be utilized without an immediately adjacent CBE. Other antigen receptor loci in mouse and humans also have large numbers of CBEs (Proudhon et al., 2015; Bolland et al., 2016), including some in Igκ and Tcrα/δ that play IGCR1-like functions (Xiang et al., 2014; Chen et al., 2015). RAG scanning in TCRδ also is restricted to CBE-anchored loop domains (Zhao et al., 2016). Similar to the proximal and distal Igh, differing V domain CBE organizations among antigen receptor loci also might function in the context of RAG scanning/loop extrusion.

### REFERENCES

Aiden, E. L., and Casellas, R. (2015). Somatic Rearrangement in B Cells: It's (Mostly) Nuclear Physics. Cell 162, 708-711.
Alt, F. W., Rosenberg, N., Lewis, S., Thomas, E., and Baltimore, D. (1981). Organization and reorganization of immunoglobulin genes in A-MuLV-transformed cells: rearrangement of heavy but not light chain genes. Cell 27, 381-390.
Alt, F. W., Zhang, Y., Meng, F.-L., Guo, C., and Schwer, B. (2013). Mechanisms of programmed DNA lesions and genomic instability in the immune system. Cell 152, 417-429.
Benner, C., Isoda, T., and Murre, C. (2015). New roles for DNA cytosine modification, eRNA, anchors, and superanchors in developing B cell progenitors. Proc. Natl. Acad. Sci. U.S.A. 112, 12776-12781.
Bolland, D. J., Koohy, H., Wood, A. L., Matheson, L. S., Krueger, F., Stubbington, M. J. T., Baizan-Edge, A., Chovanec, P., Stubbs, B. A., Tabbada, K., et al. (2016). Two Mutually Exclusive Local Chromatin States Drive Efficient V(D)J Recombination. Cell Rep 15, 2475-2487.
Bossen, C., Mansson, R., and Murre, C. (2012). Chromatin topology and the regulation of antigen receptor assembly. Annu. Rev. Immunol. 30, 337-356.
Bredemeyer, A. L., Sharma, G. G., Huang, C.-Y., Helmink, B. A., Walker, L. M., Khor, K. C., Nuskey, B., Sullivan, K. E., Pandita, T. K., Bassing, C. H., et al. (2006). ATM stabilizes DNA double-strand-break complexes during V(D)J recombination. Nature 442, 466-470.
Chen, L., Carico, Z., Shih, H.-Y., and Krangel, M. S. (2015). A discrete chromatin loop in the mouse Tcra-Tcrd locus shapes the TCRδ and TCRα repertoires. Nat. Immunol. 16, 1085-1093.
Choi, N. M., Loguercio, S., Verma-Gaur, J., Degner, S. C., Torkamani, A., Su, A. I., Oltz, E. M., Artyomov, M., and Feeney, A. J. (2013). Deep sequencing of the murine IgH repertoire reveals complex regulation of nonrandom V gene rearrangement frequencies. J. Immunol. 191, 2393-2402.
Cong, L., Ran, F. A., Cox, D., Lin, S., Barretto, R., Habib, N., Hsu, P. D., Wu, X., Jiang, W., Marraffini, L. A., et al. (2013). Multiplex genome engineering using CRISPR/Cas systems. Science 339, 819-823.
de Wit, E., Vos, E. S. M., Holwerda, S. J. B., Valdes-Quezada, C., Verstegen, M. J. A. M., Teunissen, H., Splinter, E., Wijchers, P. J., Krijger, P. H. L., and de Laat, W. (2015). CTCF Binding Polarity Determines Chromatin Looping. Molecular Cell 60, 676-684.
Degner, S. C., Verma-Gaur, J., Wong, T. P., Bossen, C., Iverson, G. M., Torkamani, A., Vettermann, C., Lin, Y. C., Ju, Z., Schulz, D., et al. (2011). CCCTC-binding factor (CTCF) and cohesin influence the genomic architecture of the Igh locus and antisense transcription in pro-B cells. Proc. Natl. Acad. Sci. U.S.a. 108, 9566-9571.
Dekker, J., and Mirny, L. (2016). The 3D Genome as Moderator of Chromosomal Communication. Cell 164, 1110-1121.
Dekker, J., Rippe, K., Dekker, M., and Kleckner, N. (2002). Capturing chromosome conformation. Science 295, 1306-1311.
Denker, A., and de Laat, W. (2016). The second decade of 3C technologies: detailed insights into nuclear organization. Genes Dev. 30, 1357-1382.
Dixon, J. R., Gorkin, D. U., and Ren, B. (2016). Chromatin Domains: The Unit of Chromosome Organization. Molecular Cell 62, 668-680.
Dixon, J. R., Selvaraj, S., Yue, F., Kim, A., Li, Y., Shen, Y., Hu, M., Liu, J. S., and Ren, B. (2012). Topological domains in mammalian genomes identified by analysis of chromatin interactions. Nature 485, 376-380.
Ebert, A., Hill, L., and Busslinger, M. (2015). Spatial Regulation of V-(D)J Recombination at Antigen Receptor Loci. Adv. Immunol. 128, 93-121.
Frock, R. L., Hu, J., Meyers, R. M., Ho, Y.-J., Kii, E., and Alt, F. W. (2015). Genome-wide detection of DNA double-stranded breaks induced by engineered nucleases. Nat. Biotechnol. 33, 179-186.
Fudenberg, G., Imakaev, M., Lu, C., Goloborodko, A., Abdennur, N., and Mirny, L. A. (2016). Formation of Chromosomal Domains by Loop Extrusion. Cell Rep 15, 2038-2049.
Fuxa, M., Skok, J., Souabni, A., Salvagiotto, G., Roldan, E., and Busslinger, M. (2004). Pax5 induces V-to-DJ rearrangements and locus contraction of the immunoglobulin heavy-chain gene. Genes Dev. 18, 411-422.
Guo, C., Yoon, H. S., Franklin, A., Jain, S., Ebert, A., Cheng, H.-L., Hansen, E., Despo, O., Bossen, C., Vettermann, C., et al. (2011). CTCF-binding elements mediate control of V(D)J recombination. Nature 477, 424-430.
Guo, Y., Xu, Q., Canzio, D., Shou, J., Li, J., Gorkin, D. U., Jung, I., Wu, H., Zhai, Y., Tang, Y., et al. (2015). CRISPR Inversion of CTCF Sites Alters Genome Topology and Enhancer/Promoter Function. Cell 162, 900-910.
Hnisz, D., Day, D. S., and Young, R. A. (2016). Insulated Neighborhoods: Structural and Functional Units of Mammalian Gene Control. Cell 167, 1188-1200.
Hu, J., Meyers, R. M., Dong, J., Panchakshari, R. A., Alt, F. W., and Frock, R. L. (2016). Detecting DNA double-stranded breaks in mammalian genomes by linear amplification-mediated high-throughput genome-wide translocation sequencing. Nat Protoc 11, 853-871.
Hu, J., Zhang, Y., Zhao, L., Frock, R. L., Du, Z., Meyers, R. M., Meng, F.-L., Schatz, D. G., and Alt, F. W. (2015). Chromosomal Loop Domains Direct the Recombination of Antigen Receptor Genes. Cell 163, 947-959.
Kim, M.-S., Lapkouski, M., Yang, W., and Gellert, M. (2015). Crystal structure of the V(D)J recombinase RAG1-RAG2. Nature 518, 507-511.
Langmead, B., and Salzberg, S. L. (2012). Fast gapped-read alignment with Bowtie 2. Nat. Methods 9, 357-359.
Lin, S. G., Ba, Z., Du, Z., Zhang, Y., Hu, J., and Alt, F. W. (2016). Highly sensitive and unbiased approach for elucidating antibody repertoires. Proc. Natl. Acad. Sci. U.S.a. 113, 7846-7851.
Lin, S. G., Guo, C., Su, A., Zhang, Y., and Alt, F. W. (2015). CTCF-binding elements 1 and 2 in the Igh intergenic control region cooperatively regulate V(D)J recombination. Proc. Natl. Acad. Sci. U.S.a. 112, 1815-1820.
Lucas, J. S., Zhang, Y., Dudko, O. K., and Murre, C. (2014). 3D trajectories adopted by coding and regulatory DNA elements: first-passage times for genomic interactions. Cell 158, 339-352.
Medvedovic, J., Ebert, A., Tagoh, H., Tamir, I. M., Schwickert, T. A., Novatchkova, M., Sun, Q., Huis In't Veld, P. J., Guo, C., Yoon, H. S., et al. (2013). Flexible long-range loops in the VH gene region of the Igh locus facilitate the generation of a diverse antibody repertoire. Immunity 39, 229-244.
Merkenschlager, M., and Nora, E. P. (2016). CTCF and Cohesin in Genome Folding and Transcriptional Gene Regulation. Annu Rev Genomics Hum Genet 17, 17-43.
Nichols, M. H., and Corces, V. G. (2015). A CTCF Code for 3D Genome Architecture. Cell 162, 703-705.
Nora, E. P., Goloborodko, A., Valton, A.-L., Gibcus, J. H., Uebersohn, A., Abdennur, N., Dekker, J., Mirny, L. A., and Bruneau, B. G. (2017). Targeted Degradation of CTCF Decouples Local Insulation of Chromosome Domains from Genomic Compartmentalization. Cell 169, 930-944.e22.
Nora, E. P., Lajoie, B. R., Schulz, E. G., Giorgetti, L., Okamoto, I., Servant, N., Piolot, T., van Berkum, N. L., Meisig, J., Sedat, J., et al. (2012). Spatial partitioning of the regulatory landscape of the X-inactivation centre. Nature 485, 381-385.
Phillips-Cremins, J. E., Sauria, M. E. G., Sanyal, A., Gerasimova, T. I., Lajoie, B. R., Bell, J. S. K., Ong, C.-T., Hookway, T. A., Guo, C., Sun, Y., et al. (2013). Architectural Protein Subclasses Shape 3D Organization of Genomes during Lineage Commitment. Cell 153, 1281-1295.
Proudhon, C., Hao, B., Raviram, R., Chaumeil, J., and Skok, J. A. (2015). Long-Range Regulation of V(D)J Recombination. Adv. Immunol. 128, 123-182.
Ran, F. A., Hsu, P. D., Wright, J., Agarwala, V., Scott, D. A., and Zhang, F. (2013). Genome engineering using the CRISPR-Cas9 system. Nat Protoc 8, 2281-2308.
Rao, S. S. P., Huang, S.-C., Glenn St Hilaire, B., Engreitz, J. M., Perez, E. M., Kieffer-Kwon, K.-R., Sanborn, A. L., Johnstone, SE., Bascom, G. D., Bochkov, I. D., et al. (2017). Cohesin Loss Eliminates All Loop Domains. Cell 171, 305-320.e324.
Rao, S. S. P., Huntley, M. H., Durand, N. C., Stamenova, E. K., Bochkov, I. D., Robinson, J. T., Sanborn, A. L., Machol, I., Omer, A. D., Lander, E. S., et al. (2014). A 3D map of the human genome at kilobase resolution reveals principles of chromatin looping. Cell 159, 1665-1680.
Revilla-I-Domingo, R., Bilic, I., Vilagos, B., Tagoh, H., Ebert, A., Tamir, I. M., Smeenk, L., Trupke, J., Sommer, A., Jaritz, M., et al. (2012). The B-cell identity factor Pax5 regulates distinct transcriptional programmes in early and late B lymphopoiesis. Embo J. 31, 3130-3146.
Ru, H., Chambers, M. G., Fu, T.-M., Tong, A. B., Liao, M., and Wu, H. (2015). Molecular Mechanism of V(D)J Recombination from Synaptic RAG1-RAG2 Complex Structures. Cell 163, 1138-1152.
Ruiz-Velasco, M., Kumar, M., Lai, M. C., Bhat, P., Solis-Pinson, A. B., Reyes, A., Kleinsorg, S., Noh, K.-M., Gibson, T. J., and Zaugg, J. B. (2017). CTCF-Mediated Chromatin Loops between Promoter and Gene Body Regulate Alternative Splicing across Individuals. Cell Syst 5, 628-637.e6.
Sanborn, A. L., Rao, S. S. P., Huang, S.-C., Durand, N. C., Huntley, M. H., Jewett, A. I., Bochkov, I. D., Chinnappan, D., Cutkosky, A., Li, J., et al. (2015). Chromatin extrusion explains key features of loop and domain formation in wild-type and engineered genomes. Proc. Natl. Acad. Sci. U.S.a. 112, E6456-E6465.
Schwarzer, W., Abdennur, N., Goloborodko, A., Pekowska, A., Fudenberg, G., Loe-Mie, Y., Fonseca, N. A., Huber, W., H Haering, C., Mirny, L., et al. (2017). Two independent modes of chromatin organization revealed by cohesin removal. Nature 551, 51-56.
Splinter, E., de Wit, E., van de Werken, H. J. G., Klous, P., and de Laat, W. (2012). Determining long-range chromatin interactions for selected genomic sites using 4C-seq technology: from fixation to computation. Methods 58, 221-230.
Stadhouders, R., Kolovos, P., Brouwer, R., Zuin, J., van den Heuvel, A., Kockx, C., Palstra, R.-J., Wendt, K. S., Grosveld, F., van Ijcken, W., et al. (2013). Multiplexed chromosome conformation capture sequencing for rapid genome-scale high-resolution detection of long-range chromatin interactions. Nat Protoc 8, 509-524.
Teng, G., and Schatz, D. G. (2015). Regulation and Evolution of the RAG Recombinase. Adv. Immunol. 128, 1-39.
Volpi, S. A., Verma-Gaur, J., Hassan, R., Ju, Z., Roa, S., Chatterjee, S., Werling, U., Hou, H., Will, B., Steidl, U., et al. (2012). Germline deletion of Igh 3′ regulatory region elements hs 5, 6, 7 (hs5-7) affects B cell-specific regulation, rearrangement, and insulation of the Igh locus. J. Immunol. 188, 2556-2566.
Xiang, Y., Park, S.-K., and Garrard, W. T. (2014). A major deletion in the Vκ-Jκ intervening region results in hyperelevated transcription of proximal Vκ genes and a severely restricted repertoire. J. Immunol. 193, 3746-3754.
Xiang, Y., Park, S.-K., and Garrard, W. T. (2013). Vκ gene repertoire and locus contraction are specified by critical DNase I hypersensitive sites within the Vκ-Jκ intervening region. J. Immunol. 190, 1819-1826.
Xiang, Y., Zhou, X., Hewitt, S. L., Skok, J. A., and Garrard, W. T. (2011). A multifunctional element in the mouse Igκ locus that specifies repertoire and Ig loci subnuclear location. J. Immunol. 186, 5356-5366.
Yancopoulos, G. D., and Alt, F. W. (1985). Developmentally controlled and tissue-specific expression of unrearranged VH gene segments. Cell 40, 271-281.
Yancopoulos, G. D., Blackwell, T. K., Suh, H., Hood, L., and Alt, F. W. (1986). Introduced T cell receptor variable region gene segments recombine in pre-B cells: evidence that B and T cells use a common recombinase. Cell 44, 251-259.
Yancopoulos, G. D., Desiderio, S. V., Paskind, M., Kearney, J. F., Baltimore, D., and Alt, F. W. (1984). Preferential utilization of the most JH-proximal VH gene segments in pre-B-cell lines. Nature 311, 727-733.
Zhang, Y., Liu, T., Meyer, C. A., Eeckhoute, J., Johnson, D. S., Bernstein, BE., Nusbaum, C., Myers, R. M., Brown, M., Li, W., et al. (2008). Model-based analysis of ChIP-Seq (MACS). Genome Biol. 9, R137.
Zhao, L., Frock, R. L., Du, Z., Hu, J., Chen, L., Krangel, M. S., and Alt, F. W. (2016). Orientation-specific RAG activity in chromosomal loop domains contributes to Tcrd V(D)J recombination during T cell development. J. Exp. Med. 213, 1921-1936.

### Star Methods

### Experimental Model and Subject Details

Mice. A 2.2-kb 5′ homology arm encompassing the VH81X gene segment sequence and containing an 18-bp scrambled mutation of VH81X-CBE that abrogates CTCF binding (FIG. 9A) and a 5-kb 3′ homology arm containing sequences downstream VH81X-CBE were cloned into the pLNTK targeting vector containing a pGK-NeoR cassette (FIG. 9B). 129SV TC1 embryonic stem (ES) cells were electroporated with this targeting construct and ES clones were screened for correct targeted mutations by Southern blotting and confirmed by PCR-digestion using the strategies outlined in detail in FIGS. 9C-9F. Two correctly targeted ES clones were injected for germline transmission following Cre-loxP mediated deletion of the NeoR gene, one of which contributed to the germline yielding VH81X-CBEwt/scr 129SV mice, which were bred to yield VH81X-CBEscr/scr mice and their WT littermates that were used for analyses. As our targeting strategy to generate the VH81X-CBEscr allele also placed a loxP sequence 642 bp downstream of the VH81X-CBEscr mutation, we generated control mice harboring only the loxP insertion, without the VH81X-CBE scramble mutation, and found that their BM pro-B cells had VH utilization patterns that were not significantly different than those of WT (Jain S., and Alt F.W., unpublished data). Primers used for construction of targeting vector, Southern probes and PCR screening are listed in Table 3. All animal experiments were performed under protocols approved by the Institutional Animal Care and Use Committee of Boston Children's Hospital.

Cell lines, v-Abl kinase transformed pro-B cell lines were derived by retroviral infection of bone marrow cells from 4-6 weeks old mice with the pMSCV-v-Abl retrovirus, as previously described (Bredemeyer et al., 2006). Transfected cells were cultured in RPMI medium containing 15% (v/v) FBS for two months to recover stably transformed v-Abl pro-B cell lines. The “DHFL16.1JH4” line was generated by transiently inducing RAG expression in v-Abl pro-B cell lines derived from Eμ-Bcl2 transgenic mice by arresting them in G1 for 4 days by treatment with 3 pM STI-571 (Hu et al., 2015). Single cell clones were screened for VHDJH and DJH rearrangements first by PCR using degenerate VH and D primers together with a JH4 primer (Guo et al., 2011) and subsequently confirmed by Southern blotting to isolate the parental DHFL16.1JH4 line (See FIG. 2A for diagrams of the DJH and non-productive VDJH alleles in the DHFL16.1JH4 line).

All mutant lines analyzed in this study (except those shown in FIG. 13A-13B) were derived from this DHFL16.1JH4 parental line or its direct derivatives by Cas9/gRNA approaches (Cong et al, 2013). The VH81X-CBEdel mutant was generated by imprecise rejoining of a DSB induced by a gRNA that targets the VH81X-CBE. The VH81X-CBEinv, VH2-2-CBEscr, and VH5-1-CBEins lines were obtained by homologous recombination-mediated repair of targeted DNA breaks introduced by Cas9/gRNA with single-stranded DNA oligonucleotides (ssODNs) as template (Ran et al., 2013). The IGCR1 deletion mutants of the parental, VH81X-CBEdel and VH5-1-CBEins DHFL16.1JH4 lines were derived via a Cas9/gRNA targeting approach based on using two gRNAs specific to sites flanking the intended IGCR1 deletion. The 101-kb intergenic deletion was derived from parental and VH81X-CBEdel DHFL16.1JH4 lines using gRNAs that target sites flanking the intended deletion. At least two independent lines were derived and analyzed for each mutation studied except for the VH81X-CBEdel. However, from the same DHFL16.1JH4 parental line, we generated an additional line in which the VH81X-CBE was disrupted by a random 13-bp insertion (not shown), and found that it had VH-utilization patterns essentially identical to those of the VH81X-CBEdel line. Rag2 was deleted by the Cas9/gRNA approach mentioned above from all of the lines analyzed by 3C-HTGTS to study chromatin interactions. The v-Abl lines shown in FIG. 13A-13B were derived by retroviral infection of bone marrow cells (see above) from Rag2−/− and Rag2−/− VH81Xscr/scr mice, and subsequently targeted for IGCR1 deletion via the Cas9/gRNA approach. Sequences of all gRNAs and ssODNs are listed in Table 3.

### Method Details

Bone marrow pro-B cell purification. Single cell suspensions were derived from bone marrows of 4-6 weeks old mice and incubated in Red Blood Cell Lysing Buffer (Sigma-Aldrich, #R7757) to deplete the erythrocytes. Remaining cells were stained with anti-B220-APC (eBioscience, #1817-0452-83), anti-CD43-PE (BD Pharmingen, #553271), and anti-IgM-FITC (eBioscience, #11-5790-81) antibodies for 30 minutes at 40 C. Excess antibodies were washed off and B220+CD43highIgM-pro-B cells were isolated (Guo et al., 2011) by FACS sorting using a BD FACSARIA™ III cell sorter.

HTGTS-Rep-Seq to determine VH utilization frequencies. HTGTS-Rep-Seq was performed and data were analyzed with all duplicate junctions included in the analyses as previously described (Hu et al., 2016). Briefly, 2 μg of genomic DNA from sorted mouse primary pro-B cells or 50 μg of genomic DNA isolated from v-Abl lines following 4 days of G1 arrest by treatment with 3 pM STI-571, was sonicated for 25 seconds ON and 60 seconds OFF for two cycles on a Diagenode Bioruptor™ sonicator at low setting. Sonicated DNA was linearly amplified with a biotinylated JH4 coding end primer that anneals downstream of the JH4 segment. The biotin-labeled single-stranded DNA products were enriched with streptavidin C1 beads (Thermo Fisher Scientific, #65001), and 3′ ends were ligated with the bridge adaptor containing a 6-nucleotide overhang. The adaptor-ligated products were amplified by a nested JH4 coding end primer and an adaptor-complementary primer. The products were then prepared for sequencing on Illumina MiSeq™ platform after tagging with the P5-I5 and P7-I7 sequences (Hu et al., 2016). Junctions were aligned to AJ851868/mm9 hybrid genome by combining all of the annotated 129SV Igh sequences (AJ851868) and the distal VH sequences from the C57BL/6 background (mm9) starting from VH8-2 as described in Lin et al., 2016. The sequence of the JH4 coding end primer used for making HTGTS-Rep-Seq libraries is listed in Table 4. In primary pro-B cells, our assay recovers D-to-JH4 as well as VH-to-DJH4 junctions; whereas in the DHFL16.1JH4 rearranged v-Abl pro-B lines, we recover VH to DHFL16.1JH4 rearrangements using the JH4 baiting primer. In the DHFL16.1JH4 lines, this primer also amplifies across JH4 on the pre-rearranged VHDJH3 rearranged non-productive allele (FIG. 2A); however, those reads are all filtered out as germline reads and are, thus, excluded from our V(D)J junction analyses.

As our experiments are done in G1-arrested cells, all de novo rearrangements should represent unique events. However, rearrangements at low but variable levels can occur in cycling v-Abl lines and can be well above background in some sub-clones (e.g. Alt et al., 1981) Therefore, after each HTGTS experiment, data were analyzed for high levels of recurrent Igh V(D)J junctional sequences suggestive of a pre-rearranged V(D)J rearrangement that likely occurred in cycling cells during culture. Then, if necessary, experiments were repeated on additional sub-clones that lacked evidence of obvious pre-rearrangements.

For statistical analyses, each HTGTS library plotted for comparison in a figure panel was normalized for by random selection of the number of junctions recovered from the smallest library in the comparison set. While normalization was done for statistical comparison, we note that relative VH utilization patterns were essentially same in normalized and un-normalized libraries. The numbers of junctions used for normalization of IGCR1del or 101-kb intergenicdel experiments was much higher than those shown for panels comparing WT and other mutant backgrounds due to the greatly increased levels of VH to DJH junctions recovered upon IGCR1-deletion or 101-kb intergenic deletion as described in main text and shown in FIG. 12A and Table 2. The numbers of junctions recovered in each replicate experiment are listed in Table 5. Data plots show average utilization frequencies±SD.

For v-Abl lines, the same WT data is shown in FIGS. 2C, 2G, 4B, 6B, all mutant lines were derived from a single WT DHFL16.1JH4 parent line. As such, several different mutants were analyzed alongside the WT control in any given experiment and the WT control was simultaneously analyzed with each mutant at least once to ensure that the WT line gave the same rearrangement pattern over the course of the entire study. Final WT averages were calculated from data collected over the course of this study. We also show the same IGCR1del DHFL16.1JH4 control data in FIGS. 5B, 12B, 14A and 14B, as we used the same gRNA strategy, respectively, to generate IGCR1del, IGCR1del VH81X-CBEdel, and IGCR1del VH5-1-CBEins lines from the same common DHFL16.1JH4 ancestor line (as described above). The IGCR1del data is plotted as the average of experiments done along with IGCR1del VH81X-CBEdel or IGCR1del VH5-1-CBEins lines.

The non-productive fraction of VHDJH reads obtained from C57BL/6 pro-B cells shown in FIG. 8A-8E were extracted from data in a prior publication (Lin et al., 2016).

### 3C-HTGTC

3C libraries were generated as previously described (Splinter et al., 2012; Stadhouders et al., 2013). Briefly, 10 million cells were cross-linked with 2% (v/v) formaldehyde for 10′ at room temperature, followed by quenching with glycine at a final concentration of 125 mM. Cells were lysed in 50 mM Tris-HCl, pH 7.5, containing 150 mM NaCl, 5 mM EDTA, 0.5% NP-40, 1% TritonX-100 and protease inhibitors (Roche, #11836153001). Nuclei were digested with 700 units of NlaIII (NEB, #R0125) or MseI (NEB, #R0525) restriction enzyme at 370C overnight, followed by ligation under dilute conditions at 160C overnight. Crosslinks were reversed and samples were treated with Proteinase K (Roche, #03115852001) and RNase A (Invitrogen, #8003089) prior to DNA precipitation. The 3C libraries were sonicated for 25 seconds ON and 60 seconds OFF for two cycles on a Diagenode Bioruptor™ Sonicator at low setting. LAM-HTGTS libraries were then prepared and analyzed as described in “HTGTS-Rep-Seq to determine VH utilization frequencies” section (see also Hu et al., 2016) and data was aligned to AJ851868/mm9 hybrid genome as described in Lin et al., 2016 with an additional modification in which Chr12 coordinates from 114671120 to 114734564 in the AJ851868/mm9 hybrid genome were replaced with CCCCT to incorporate the DHFL16.1 to JH4 rearrangement for aligning data obtained from the DHFL16.1JH4 rearranged v-Abl pro-B lines. When using the iEμ bait, we also detected interactions with distal regions beyond VH1-2P in the DHFL16.1JH4 rearranged v-Abl pro-B lines due to close linear juxtaposition of this region to iEμ owing to the VHDJH rearrangement of VH1-2P on the non-productive allele. These interactions were not detected in the unrearranged v-Abl pro-B lines or primary pro-B cells as evident from data deposited in GEO database. The primers used for making 3C-HTGTS libraries are listed in Table 4. Data were plotted for comparison after normalizing junction from each experimental 3C-HTGTS library by random selection to the total number of genome-wide junctions recovered from the smallest library in the set of libraries being compared. However, chromosomal interaction patterns were very similar in normalized and un-normalized libraries.

Electrophoretic Mobility Shift Assay (EMSA). EMSA was performed with oligos (shown in FIG. 9A) using the LightShift™ Chemiluminescent EMSA kit from Thermo Fisher Scientific (Catalog #20148) as per manufacturer's protocol. 2 μg of anti-CTCF antibody from Millipore (Catalog #07-729) was used to detect super-shift.

ChIP-seq, CTCF and Rad21 ChIP-seq data were extracted from Choi et al., 2013 (GEO: GSE47766). Pax5 and YY1 ChIP-seq data was extracted from Revilla-I-Domingo et al., 2012 (GEO: GSE38046) and Medvedovic et al., 2013 (GEO: GSE43008), respectively. The ChIP-seq data were re-analyzed by aligning to mm9 and ChIP-seq peaks were called using MACS with default parameters (Zhang et al., 2008).

### Quantification and Statistical Analysis

An unpaired, two-tailed Student's t-test was used to determine the statistical significance of differences between samples, ns indicates p>0.05, * p≤0.05, ** p≤0.01 and *** p≤0.001.

### Data and Software Availability

The Gene Expression Omnibus (GEO) accession number for the datasets reported in this paper is GEO: GSE113023. Specifically, the accession numbers for pro-B-HTGTS-Rep-Seq, DHFL16.1JH4-HTGTS-Rep-Seq and 3C-HTGTS datasets reported in this paper are GEO: GSE112781, GEO: GSE112822 and GEO: GSE113022, respectively GSExxxxx.

TABLE 1
Proximal V_{H} usage in WT and mutant primary pro-B cells and v-Abl transformed D_{H}FL16.1J_{H}4 pro-B cell lines.
Primary pro-B cells

| | Total VDJ_{H} | | | | | | |
|---|---|---|---|---|---|---|---|
| Genotype | Junctions^{a} | V_{H}5-1 | V_{H}81X | V_{H}2-2 | V_{H}5-4 | V_{H}2-3 | V_{H}5-6 |
| WT | 10,000 | 40 ± 27 | 947 ± 61 | 560 ± 39 | 300 ± 57 | 218 ± 22 | 163 ± 15 |
| V_{H}81X-CBE^{scr/scr} | 10,000 | 26 ± 8 | 18 ± 1 4 | 1,163 ± 182 | 475 ± 47 | 327 ± 26 | 227 ± 30 |

D_{H}FL16.1J_{H}4 rearranged v-Abl pro-B cell lines

| | Total VDJ_{H} | | | | | | |
|---|---|---|---|---|---|---|---|
| | Junctions^{a} | V_{H}5-1 | V_{H}81X | V_{H}2-2 | V_{H}5-4 | V_{H}2-3 | V_{H}5-6 |
| WT | 3,500 | 87 ± 10 | 1,579 ± 111 | 791 ± 65 | 372 ± 15 | 230 ± 16 | 123 ± 21 |
| V_{H}81X-CBE^{del} | 3,500 | 77 ± 8 | 18 ± 2 | 1,807 ± 8 | 587 ± 21 | 390 ± 22 | 214 ± 18 |
| IGCR1^{del} | 100,000 | 1,385 ± 269 | 77,138 ± 2,391 | 15,194 ± 1,741 | 3,845 ± 1,277 | 1,911 ± 636 | 223 ± 143 |
| IGCR1^{del} V_{H}81X-CBE^{del} | 100,000 | 3,147 ± 301 | 520 ± 224 | 51,419 ± 4,765 | 28,361 ± 3,496 | 10,334 ± 782 | 4,891 ± 1,978 |
| Intergenic^{del} | 100,000 | 0 | 58,364 ± 6,671 | 29,742 ± 6,735 | 8,826 ± 1,218 | 2,459 ± 524 | 306 ± 51 |
| Intergenic^{del} V_{H}81X-CBE^{del} | 100,000 | 0 | 62,239 ± 16,210 | 23,133 ± 8,979 | 10,282 ± 5,221 | 2,847 ± 1,634 | 609 ± 337 |
| V_{H}81X-CBE^{inv} | 3,500 | 79 ± 28 | 803 ± 13 | 1,232 ± 141 | 668 ± 152 | 342 ± 38 | 181 ± 45 |
| V_{H}2-2-CBE^{scr} | 3,500 | 124 ± 33 | 1,701 ± 117 | 8 ± 3 | 665 ± 58 | 445 ± 32 | 181 ± 33 |
| V_{H}5-1-CBE^{ins} | 3,500 | 1,994 ± 102 | 641 ± 32 | 371 ± 44 | 160 ± 55 | 99 ± 5 | 59 ± 21 |
| IGCR1^{del} V_{H}5-1-CBE^{ins} | 100,000 | 82,753 ± 655 | 13,987 ± 434 | 1,778 ± 45 | 957 ± 74 | 328 ± 76 | 109 ± 19 |

^{a}Refers to the total number of VDJ_{H} junctions to which each replicate library was normalized to, n ≥ 3 (see Figures for details).

These averages were derived from three or more independent libraries generated from at least two independently derived mutant clones (except for V_{H}81X-CBEdel lines, see STAR Methods for details), which gave essentially indistinguishable patterns of V_{H} utilization.

TABLE 2
Average Number of VDJ_{H} junctions recovered from
D_{H}FL16.1J_{H}4 rearranged v-Abl pro-B cell lines

| | Average number of VDJ_{H} junctions |
|---|---|
| Genotype | recovered per 120,000 aligned reads^{a} |
| WT; n = 3 | 3,288 ± 583 |
| Intergenic^{del}; n = 3 | 94,464 ± 7,056 |
| Intergenic^{del} V_{H}81X-CBE^{del}; | 92,490 ± 5,877 |
| n = 5 | |
| IGCR1^{del}; n = 3 | 101,552 ± 8,140 |
| IGCR1^{del} V_{H}81X-CBE^{del}; n = 4 | 93,663 ± 6,360 |

^{a}Aligned reads include all D_{H}FL16.1J_{H}4 reads as well as V_{H} to D_{H}FL16.1J_{H}4 junctions

TABLE 3
List of primers used for the generation of mutations in mouse ES cells and
D_{H}FL16.1J_{H}4v-Abl pro-B cell lines
Oligos related to targeting V_{H}81X-CBE scramble mutation in ES cells

| | |
|---|---|
| 5′ homology arm-F | TATAACTCGAGAACAGGAACCCTAAAACGGAACª |
| | (SEQ ID NO: 14) |
| 5′ homology arm-R | TTAAACTCGAGAAACCAGGCAAGAGGAGTCCATª |
| | (SEQ ID NO: 15) |
| 3′ homology arm-F | ACAACGTCGACAGCTCTATAGAGATTCTCTCTAAAAGT^{b} |
| | (SEQ ID NO: 16) |
| 3′ homology arm-R | TAATAGTCGACAGAATGAGTCCAGCACTCTC^{b} |
| | (SEQ ID NO: 17) |
| Probe A-F | TTTGAATTAGCATTCACCATACTTAA (SEQ ID NO: 18) |
| Probe A-R | GTGTTTCAGTCATATGCAGAACATTC (SEQ ID NO: 19) |
| Neo probe-F | AGTATCCATCATGGCTGATGCAATG (SEQ ID NO: 20) |
| Neo probe-R | CTCAGAAGAACTCGTCAAGAAGGC (SEQ ID NO: 21) |
| P1 (FIG. S2E) | CCTGTGAATCCAATGAATACGAATTCC (SEQ ID NO: 22) |
| P2 (FIG. S2E) | AAACCAGGCAAGAGGAGTCCAT (SEQ ID NO: 23) |

Cas9/gRNAs and ssODNs used to generate mutant v-Abl pro-B cell lines

| | |
|---|---|
| V_{H}81X-CBE deletion | caccgTCCAGGACCAGCAGGGGGCG (SEQ ID NO: 24) |
| (gDNA) | |
| V_{H}81X-CBE inversion | caccgAAACCTCCTGCAGAGCATCC (SEQ ID NO: 25) |
| (gDNA) | |
| V_{H}81X-CBE inversion | TGAAGGTGGGTTGGAGGTTGGAGACAATTTTACAGGCTGTA |
| (ssODN) | ACTCTGTATTTCACAACTCcagagcatccaggaccagcagggggcgcggagag |
| | cacacaCAGGAGGTTTTAGTTTGAGCTCACAGTAACTTTTGCTC |
| | ATTGTGTGTCTTGCACAGTAAT (SEQ ID NO: 26) |
| V_{H}2-2-CBE mutation | caccGACCCTGGGATGTCATGGTT (SEQ ID NO: 27) |
| (gDNA) | |
| V_{H}2-2-CBE mutation | AAACACAGTGAGGGAAGTCCATTATGAACTTGAACAAAAA |
| (ssODN) | TTTCACTAGAAAGATGATCAcgcgacgagaaggctagcaggcggCAACC |
| | ATGACATCCCAGGGTCACTGCAGAATCTAGGTCAGCTGGCT |
| | CCATTTTTTGTTTA (SEQ ID NO: 28) |
| V_{H}5-1-CBE insertion | caccGTGTTCTCTTCGCCTCCTTC (SEQ ID NO: 29) |
| (gDNA) | |
| V_{H}5-1-CBE insertion | CAGCACTCTCTTTCCTCCAGGTCTTCCTGAATGGGCTGTAAC |
| (ssODN) | ACTCAGTAACTATTAGATTTGAGaGaTCTCactGCCcCCTTCTG |
| | GTCAGGGGGTCCTTATAGGAGGTTTGTGTTTGAGCTCACAGT |
| | AACATTCACTCACTGTGT (SEQ ID NO: 30) |
| Intergenic deletion | caccgTGTCAACTAACCTGTACACC (SEQ ID NO: 31) |
| (gDNA) up | |
| Intergenic deletion | aaacGGTGTACAGGTTAGTTGACAc (SEQ ID NO: 32) |
| (gDNA) down | |
| IGCR1 (gDNA) up | GGAAAACTCTGTAGGACTAC (SEQ ID NO: 33) |
| IGCR1 (gDNA) down | TGGGACATGTAAACTGTAAC (SEQ ID NO: 34) |
| Rag2 (gDNA) up | GAATAGGTCTTTTATCTGAA (SEQ ID NO: 35) |
| Rag2 (gDNA) down | GAGCAATATACCTGAGTCTG (SEQ ID NO: 36) |

TABLE 4
List of primers used for HTGTS-Rep-Seq and 3C-HTGTS analyses
HTGTS-Rep-Seq primers

| | |
|---|---|
| J_{H}4 Coding end bio primer | /5BiosG/CCCTCAGGGACAAATATCCA (SEQ ID NO: 37) |
| J_{H}4 Coding end nested primer | CTGCAATGCTCAGAAAACTCC (SEQ ID NO: 38) |

3C-HTGTS primers

| | |
|---|---|
| V_{H}81X Bio primer | /5BiosG/AAATAGAAGATGAAATGGAAGATTTGAAG |
| | G (SEQ ID NO: 39) |
| V_{H}81X Nested primer | TGAGAAACACCAATATTGTCAACTAACC (SEQ ID |
| | NO: 40) |
| V_{H}2-2 Bio primer | /5Biosg/AAGAGGAGGGGGAGAGGATG (SEQ ID NO: |
| | 41) |
| V_{H}2-2 Nested primer | TTGTAAGGTAAACGAGGAATGGG (SEQ ID NO: 42) |
| V_{H}5-1 Bio primer | /5Biosg/AGGAAAGAGAGTGCTGGACTCATTC (SEQ |
| | ID NO: 43) |
| V_{H}5-1 Nested primer | GCCTCTCTACAGATGTTATCTTTACAAG (SEQ ID |
| | NO: 44) |
| iEμ Bio primer | /5BiosG/GGTTATGTAAGAAATTGAAGGACTTTAGTG |
| | (SEQ ID NO: 45) |
| iEμ Nested primer | CTCTATTATTCTTCCCTCTGATTATTGG (SEQ ID NO: |
| | 46) |
| D_{H}Q52-J_{H}1 Bio primer | /5Biosg/CTCAAAACAGTCGCTAAAGTTCTCG (SEQ ID |
| | NO: 47) |
| D_{H}Q52-J_{H}1 Nested primer | GAGGTCCATCTGTCATTCACTTGTG (SEQ ID NO: 48) |

TABLE 5
Number of VDJ_{H} junctions recovered from
each replicate HTGTS-Rep-Seq library

| | |
|---|---|
| | Total number of VDJ_{H} |
| Genotype | junctions recovered |

Primary pro-B cells

| | |
|---|---|
| WT-1 | 16,878 |
| WT-2 | 10,970 |
| WT-3 | 14,901 |
| V_{H}81X-CBE^{scr/scr} -1 | 12,048 |
| V_{H}81X-CBE^{scr/scr} -2 | 11,681 |
| V_{H}81X-CBE^{scr/scr} -3 | 13,044 |
| V_{H}81X-CBE^{scr/scr} -4 | 15,708 |

D_{H}FL16.1J_{H}4 rearranged v-Abl pro-B cell lines

| | |
|---|---|
| WT-1 | 3,965 |
| WT-2 | 3,890 |
| WT-3 | 5,301 |
| V_{H}81X-CBE^{del} -1 | 7,938 |
| V_{H}81X-CBE^{del} -2 | 8,341 |
| V_{H}81X-CBE^{del} -3 | 5,784 |
| V_{H}81X-CBE^{inv} -1 | 4,098 |
| V_{H}81X-CBE^{inv} -2 | 5,024 |
| V_{H}81X-CBE^{inv} -3 | 3,386^{a} |
| V_{H}81X-CBE^{inv} -4 | 3,105^{a} |
| V_{H}2-2-CBE^{mut} -1 | 7,647 |
| V_{H}2-2-CBE^{mut} -2 | 5,977 |
| V_{H}2-2-CBE^{mut} -3 | 3,560 |
| V_{H}2-2-CBE^{mut} -4 | 4,070 |
| V_{H}5-1-CBE^{ins} -1 | 16,190 |
| V_{H}5-1-CBE^{ins} -2 | 8,551 |
| V_{H}5-1-CBE^{ins} -3 | 4,125 |
| V_{H}5-1-CBE^{ins} -4 | 4,691 |
| Intergenic^{del} -1 | 100,956 |
| Intergenic^{del} -2 | 112,870 |
| Intergenic^{del} -3 | 164,533 |
| Intergenic^{del} V_{H}81X-CBE^{del} -1 | 356,626 |
| Intergenic^{del} V_{H}81X-CBE^{del} -2 | 287,723 |
| Intergenic^{del} V_{H}81X-CBE^{del} -3 | 110,240 |
| Intergenic^{del} V_{H}81X-CBE^{del} -4 | 102,639 |
| Intergenic^{del} V_{H}81X-CBE^{del} -5 | 332,151 |
| IGCR1^{del} -1 | 105,382 |
| IGCR1^{del} -2 | 125,243 |
| IGCR1^{del} -3 | 123,309 |
| IGCR1^{del} V_{H}81X-CBE^{del} -1 | 250,102 |
| IGCR1^{del} V_{H}81X-CBE^{del} -2 | 238,043 |
| IGCR1^{del} V_{H}81X-CBE^{del} -3 | 125,197 |
| IGCR1^{del} V_{H}81X-CBE^{del} -4 | 100,589 |
| IGCR1^{del} V_{H}5-1-CBE^{ins} -1 | 144,695 |
| IGCR1^{del} V_{H}5-1-CBE^{ins} -2 | 226,079 |
| IGCR1^{del} V_{H}5-1-CBE^{ins} -3 | 220,639 |

^{a}These libraries were pooled together and then 3,500 VDJ_{H} junctions were randomly extracted from the pool and treated as one library to calculate the average data

### Example 4

SEQ ID NO: 13. Sequences and deletion strategy for Mouse Cer/Sis element (-6.7 kb region on mouse chr6): CRISPR/Cas9-sgRNA1 (GCTCCTGAAGAGCTTAAGTT (SEQ ID NO: 49)) and CRISPR/Cas9-sgRNA2 (GAGGAATCTATGTCCTGGAT (SEQ ID NO: 50)) are depicted in bold font, with the PAM sites in italics. The ˜650 bp Cer (HS1-2) (bp 860-1529 of SEQ ID NO: 13) and 3.7 kb Sis (HS3-6) (bp 3562-7288 of SEQ ID NO: 13) elements are underlined with single and double lines, respectively. Shown in double parenthesis in the following sequence are, in order, i) the CBE1 of Cer element (reverse orientation, pointing to Vk segment), i) the CBE2 of Cer element (reverse orientation, pointing to Vk segment), iii) the CBE1 of Sis element (Sense-strand orientation, pointing to Jk segment), and iv) the CBE2 of Sis element (Sense-strand orientation, pointing to Jk segment)

(SEQ ID NO: 13)
Vks...GAATAAAAGCAGAAACTAATGAAAAATGTGGTTATAAAGTGAATAAAACTGTGATTG
AAATATCTTTCTCTTGAAAAGGATCATTAAAACAGATGAATATTGAGCTATTTAAAGGTAAA
ACATGCCAAAAATCATGTTATGAAGGAGCAAAGAGAAAACAACTGTATCTATAGCTCCTGA
AGAGCTTAAGTTTGGAGGAGTGTGTCCT
GCTTTTAAAGAGGTAGAACCATGCTGTAGATGAAGCCCATGATGTTCTGTGAAAAGAGAAG
TAACCCTGACTCCAGAAGATGTGTTCAACTGGAAAAGATCAATAATCAAAGATCGTAAAAC
AATTGGGAGAGACCCACCATCCCCTCCTCTGTGGGAAAGTTCAAGGTCATTTTCTTGAAAAG
TTCTAGCATATGTTTTTGGAGTAGTAGTAGTTGTTGCTGTTGTTGTTGTTGATGATGATGATG
ATGATGTTGTTGTTGTTGTTGTTGTTATATAAACCTTCTTTGGAGCATAGAAAACTACAAAAA
CAGAAACAAAAAACAAAAAAAAATATCTATTTCAGATAACCTATATTCAATACAGCTGCATT
AATGAGGCAATTTATCATCAATGAAGCATCACCTATTGTTGATTTGTTAAAGATTATTTATCT
TCAATATAAGTAAAAGCCTGATAACTGGCCCTGTTGACTGTGGCTTTTACTGCTGTTTCTCTG
TGCTGAAACTATCCATACAAAATAGAAATAAAGTCTGAAAAGTCAAAAAAAACACAATGTT
CTGATAGTTGGAAACCGTGTGTATATGTGGGGTGGGGGAGGGGGTAATGCTCATAAATGTGT
GACAGAGAGATAGGGGAAGAGGAGAAAGACAGATCTTCTAAAAACAACAGTCTGGTCCCAT
TATGGGGGTGGAGACCTGGCCAAATTGAGATCTCTGCTTTTGTTTGCAGGACAGTTCTGTGA
CCCATGACTGGGCCTCTGTAGACTTGCCGCTTATACAA((CACTGCCATCTGCTGATAC)AGC
ATTAGCACCCTGACTTGCTCTGGTGATAAACTGGAGGCACTGTGAGATCATTTCCTTGTCACT
GTTTCCTGTGCCACACCCATTCATATGTACTAGAAATAGTCTGAGAAGAAAAAGACGTTCAG
ATAGGAAGGGAGCATGTAATGTACCTATATATCTACATAGATACTTACTCAAGGGGAGGGA
GGGTTTGGTGTGTGTGTGTGTGTATCTCCCGTGCACACACACACACACGAAAAAGTTGGAGA
GGAAAGATTTTTTTTTTAAACAACAGTCTGATCCCATTATAGAGGTGGAGACCTGACAAGAT
TCAGATCACTGGCTTTGTTTGCAGGCCAGCTCAGTGACCCATGATCGGGCCTCCGTAGGCTC
ACTGCTTATACAG((CACTGCCATCTGCTGACAC))AGCTTCTCTGTTGACACAGCTTCTGCCCC
CTGCCATGCTCAGATAATGAGCTGTTCATTGGCTCTGTGAGATCGATTCCTTTTCATTGCTTT
CATTTTTGATATCTAAACAATGTTTCTACAATTCAGAGACACAAACAAATTGTATAAATAAC
TTCAATTTTACAAGTTAACATTTTCCACCTTTTACTGGTATCAAACGGCTTGCCGTGGTTCTG
ACCTGCCAAGATAGGGAGTAAAGCTCTCTTTGGTCTCTAGTCCCAGGCCTTGGAGTTCCAAA
AGCCTGGGTTTGGAGGGAGTCCCAGAAGTTTACAGCTCCAAGCCCTGAGAGCTAGAGGCCT
ACTGTTCCAGGTTTTGAAATCCAACAGGATGACTAGGGAGAGGTGGCTCAGTGGTTAAGATG
TGCCCTGCTTTTTCAGAGGATGTAAGTTCAGTTCCTAGCACCCATATCAGGCAGCTCACATGC
AGTTACATATAACAGCCTGTAATTTCATCTCCAAGGATCTGAACATCTCTTCTGGCCTCCTCA
AGCACTGTGTTCACATGCATGCACAGTGTGTGTGTGTGTGTGTGTGTGTGTGTGTGTGTGTGT
GTGTGTGTGTACTATATATACACACATATATAGTAGAGTGAGTTGAGCTGGTAGCCTGGGGT
GTCTGACACCTTGGCCCTTTGACAGTTCCTTAGAAATCTCCCAGTACCAGGGCCAGAAGTTT
CTTGTCTTCAGCAGCTGTCTCTATTGCCTCTGCTCCTCCTCATCTCTACCACAGCCTTTTGATG
TCACTGCCGATGTCACCAAGGACACTTCCTTCACCACTGACATTGCCTTCATTGTCCCTGCTT
CCTTCCTTTCCTCATGTTACCAGCTCAACTCACTCTACTAATGATAAAACGCAAAAATAGGC
AAGACCGGGCCTTTTATTGCAACTTAATGCTTCAGCTTCAACACCAGAGAGCAACATCTAGC
TGGTATCTCCAGGTTAACTTGCAGAGTGAACCAAGCAGAGCACTTATATAGCCAGAGTGGG
AGTGTGTCCAGTGTGTGTGCACGTGGCAGTACATCACACCAATGAAGAACAGTATTCTCACC
AAGCATGAAGGGCTGCACCTGTCCCAATCACAGCAGTCCCTCAGACACTCAGAATGAAGTC
ACACACCTTCAGACTGCCTTCTTGACTGATATTCCTTCATTCAAGGCCATACATAGCTCCATA
GGATGAAGCAGTCTTAATTTACCATTCAAATCATCCAAGCTATCAGGAAAATTGACATTAAG
AAACACATCTACAATAAAGTTGTCATTCACAGTGATITAACAATAGGTTAAGAATGCATAGC
TCCTGCCTCAGTGATCCCATGAGTAATGGGCCTCCTAATATGACTGTTTTACTTATCCCACCA
TCCAACCACCCTTGGCAACCACCACACACATCTCTTTGACATGTGTGTATGAGATATTTTAAT
CATATTCAAGGCTGATTACCTTCTCTTATCTCCCTCACATTCTAACTACATCCCTGCTAACTTT
GTGTCCGTTTTTAAGGTTTATTTTTATTTATGTGTATGTGTGTGTGTCTGTTATGGACCATGAG
AATGTGTCCAATCGCCTGGACCTGGAGTTACAAACAGTTGTGAATTGCCTGCTGTGGGTGCT
GGGAATCAAATTCAGGCCCTCTGCAGGAGCGTCAAGTGTTCTTGGCCGCTAAGCCACCTTCC
CACAACCCCACACACTTTTTGATAACCCGTTAGCTGAGGTAACTTACATAAACTCGGGTTAG
GGGTTATTTAACAAAGCATGATCAACTTAGCAGTGGGTATATCACTGAAGAAACTTTTGTTC
CCTCCCCTAGCAACTATTAACAGCCAAAAGCTGTTTAGAAAAGGGTAGGGGCCTTAAAAGC
CCCAATTAACCTAATTAGAAGTCCTTAACTGACTATAAATCTTAAAGGAAAAGAAGAGCCTC
ATAAGCCCCTGCCCTGCCCGTGATGGAATATTGGCAGGCCAGATCTTCTGGTTGTGAGAAAG
TAATCAAGCTGCTCTGAGTCCGTGAGTGCAACAGCCATGTCTGATTCCCAAGGATGGTGCAC
AAAGCCCCTTTCCTTCCACCAACTCTGTGTTCTTTCTACCCCTTCTCCTGCAGTGTACTCTGAA
CCTTGTATGGTGATGACATAAACGAACCATTGATGACTGAGCACTCAATCATCCCTTATTTTC
AGCGCTTTGACCCGTTATAAGTCTCTGCATAAAACACACACACACACACACACAAGGGAGG
AGGGTCTGAGCAACACTAATCTATAGGTTTTCAACCTAGCAATATTTACAAGGCCATTCAAA
AAACAAACCATGTCCATTTAGTGAAACAACAGCAGTAAGTTTCCCACTAGGGCCTTTAACCA
CACCCCCATAAGCCTTTAACCAGGTTTACAGTAGCAGGAATGAATACTGTGGAGTGGACCTC
AAACCCAATCAGAAAATGGTTGGTTACCCCCATTCTAGCCATACCACTATTGCTCCAGTGGG
CATATCTTACCTGCCGTGGGGTCGATCCAATAGGGCACAGGGTCTATAGCTAAATGCAACTG
CTGACTAGCTCCTTCACCCAGAAGACTGTGCACCATCGTCTGGCACTGTGAAATCCACCCAG
CTGAAAGGAAGCCTCCAATCGGCTCCATCTGGGCTTTTCCATGGTCTACAACCAGGAGCATG
GTGTATCCAGCAATAGGGTCTTAGCATCTAGAAATTAGCTATGGTAATTGCCTATATTGTTTG
AAGGACCTTAAGGACCTCAATGACCAACATATAGCATGGAATCTCACCCCTGGCACCAGGA
TTTTTATTTAATAACCTATGGCTTCCGGGAGCAGCTTTATCCTCCCATGCAGGGTACCTCACA
CCAACTCCTTTTTATTAATTGTACGTTAAATCACTTGCAAAGTAGTATTCTTCCTTACGGCTTT
TTCATGCACCCTCACGCAGCTTTGAATGGCCATCTCTCCCCCCTCTCCTCTTTCCCATTTTTCT
GCACTACATTCCTACTTCCTACAAAATTTGTCCTAAACAGTTTTTCCTTTCTCTCCACCATTGT
CCCTTCCAAGATTCCTAGATTTTGGTTACCTTAAATGCCAACAAGGTACAATTTTCAGAGGCT
TTACAGTAACAGAAAAAAAAGAGGTTACAAGGTACTTTTCAAATTTATTGATGGGCACAGG
AGTGCAGGTTAAAGCAAAGTGGGGAACCTCTGCTACAGACCTCGGATGCTATCTGACGGTCC
CAGTGTTTGCCGTGAGGATGCTGCTCGGCCAACAACTCAAGTCAGGATGAGTTGGGATCTGT
TCTTGTATTCCAAAGGATTTACCTAACAGTCACAAAGATGATAGGTCACAGACGGCAGTAAA
TGGCCTCAAGTAGCAGTTAA((TGGCCACTTGAGGGAGCTA))AAGATAACTTGTCTCTGGGCC
TGCACAGATTCCACCCCTCCACAGTCACTGAAGTTCTTTATTATCATTATTGTTGTTGCTGCT
GTTGTTGTTGTTGTTTTATATCCATAAATGTTGCCGCCCCCGCCCCCAGCCTCCCTTTGCAGA
TTTCTTCCCCAACACCCCCTTAGCTTCTAAGAGGGTACCCCCACCCCAGTCATCCCGCTTCCC
TCAGGCATCAAGTCTTTACAGGATTAGCTCATGCTCTCCCACTGAAGCCAAACAAATATGTC
TGCTACATATGTGCGGGGGAGGACGGGGGGGGGGGGGGGGACTCGGACCAGCCCATATATG
CTCTTTGGTTGCTGGATTTCTCTCTGGGAGCTCTCAGGGGTCTGGGTTAGATGACACTCTTCA
TCTTCCTATAGAGTTGCCATCCCTTCCTTTCCTTCAGTCCTTCCCCTAAGTCCTTCCCCTAACT
CTCCCATAGGGGTCCCCGACTTCAGTTCAATGGTTGGCAGTAAATATCTGTCTCAGTCAAGG
GCTGGTAGAGCCTCTCAGAGGACAGTCATCCAGGCTCCTGTCTGCAAGCACAACGTAGCATC
AATAATGGCGTCAGGGTTCTAATCGGTGATTCAGCCTTTGTAAAGTGGTCAACGTAAGGTGC
AGGTTCTTGGGGAGGGACTTGAAGGGGACACGAGGACTTTAATTCACATGGATAAAATAGA
AGACTGCCTCTATGAGAAAGGTGAGTCTGTGGACTAAATGGATTCTTTCCCGCAGAGAGAAA
TAGAGGAAGAATTTCAGATGCTCATTTTAAAGATAAAAGAATACTTGAAAAGAAGGGGGGG
TGGGAGGAAAGTATGACAGAGAAATCAGCTAAATGCTGCCCCCAGCTTACACTTCCTTAGA
AGGGAAAGGGAAGGGAAAGCTACTCCTGAAAGAAAAGCTAACCGAAGCAGAGCAGTCCCA
CCCTCAAGACAGGCACAGAGCTAGCTCTCACATGCTAAAGTACAGATGCAGAAACCTCTTGC
ATTGGGATCAGCCTTGGATAAAAATAAGTCGGTGAAAGACAGACTGCAAAGCTCAATG((TG
GCCAGCAGAGGCCCCTA))GTCAGCAACAAGGAAAACTCTCACGCTAACCAGACAAACAATA
CAGACTCAGCAAAAACATAAACGGAAGGATGTGCCCACAAGTTCACCTGACCCTCTTCCTCC
GTGAGTGTGCTTTTCTGAAGAGGCAGCTCCAACACTGCCTCACATCTTCCTCTCTATTGTTTT
CTTTGTGTATTCCCCCACAATACTCGCTTAGCAGGATTTTTACTGTATGTATTTGGGGTGGAT
ATATGTGTGTGTGTGTGTGTGTGTGTGTGTGTGTGTGTGTGTGTGTGTGTGCATGTGTGTGTG
TGTGTGTGTGTGTGTGTATTGTTATCTCTTTTCATACAATCATTTAATTTTGTTCGTGCGTTTT
TTCAGTTTAGAGCAGGTTTTTTTCCTTAGTTTCTTTCTTTTTTCCTTGTTTACTCCTGTGTCCCT
TACACATACACACACGCACACACACACACACACACACGTATTCATACTTCTAATTGTTTTAT
ACTTTTCTTAAGTTTTACCTTTTTCTCTTCTAGTTTTTGGTTGTCAACGCTTTCATTATTGTTAA
GTCTTTTTTTTCCCTACTTTTCCTTTTTCCCAAGTCTAGAAAAAGAAACAGACAGTGAAATAA
AAAAGGAATTGAGAACTCTAAACAGACTTCACAAGAGAAAATCCTCTTCACTCCATTTTATA
ATCAGAAAATTAAAAAAAAAAAATGTTAAGCAAAGAACAAATGTTAGGAACCGTAGGGGG
ACACCAGCTCATGCACGGGACACAAATTCCAGAGCACACAAATCCTCCCCTCTGCGGTCCTA
AAAGCCAGGAAAGTACGAAATGATGCCCTTCAATTCGGAAAGTAAATACCATCTAACCACG
CTTTAAATTGATAGCAAAGCTACTCGTGTAACAAGCAATCTATAAGTGAGTTCGTGACTGCC
AAGACTACACTACAAGATAGTTTTTAAAATTCTTTACAGAAAAGGAAGAATGACACCGGCC
ATCACAGAGGCACAGGAAAGACTGGGTTTCATGA [symbol] [symbol] TGGGAG
AAATAACGTGTGAAATGT
TGTACTAAAAAACACAATCTTCAGATTTAATGCTATCACCATCATGGTTCTAGTGACATTCTT
TACAGAACTAGAAAGATAATACTAAGCTTGTATGGAAACACAAAAGACCATGAGTAGGCTA
AGCAGGACAAACCCTACATCACATCACATCACATCACATCACTGAAACTCACATTATCCCAC
ATAAGTGTAATGACAAAGAGAGTAAGGTGCATCCACAAAAGCAGACAGAGAACAATGAAC
TGGAAGAGGGCCCATAACCTGCACTTCTGTGGA...Jks

### Example 5

Current vaccine strategies to elicit the most effective broadly neutralizing antibodies (bnAbs) against HIV-1 are based on sequential immunizations with separate immunogens that target B cells expressing precursor and intermediate forms of the bnAb. Mice expressing human bnAb precursors have been used to assess the preclinical efficacy of candidate immunogens. Commonly used mouse models generated via conventional germline human IgH and IgL variable region exon knock-in technologies have well-known limitations related to the production of a monoclonal set of primary B cells. To avoid this issue, a recent study utilized mice engineered to contain fully human immunoglobulin (Ig) variable region loci that can generate complex primary B cell receptor (BCR) repertoires by V(D)J recombination. However, due to the relatively small size of the mouse B cell compartment, the BCR repertoire of such mice is far smaller than that of humans and, correspondingly the chance of generating B cells expressing an appropriate bnAb precursor is far lower than in humans. To circumvent the shortcomings of these mouse vaccine models, we have described a new type of mouse vaccine model for the potent VRCO1 class of HIV-1 bnAbs, based on a strategy that allows the precursor human Immunoglobulin heavy (IgH) chain variable region exon for this bnAb to be developmentally assembled via V(D)J recombination and to dominate the IgH repertoire of the mice. In this VRC01-rearranging model, most individual B cells express one of a multitude of different variations of the potential VRCO1 precursor IgH chain, providing much more human-like precursor VRC01 repertoire. Indeed, sequential immunization induced affinity maturation of VRC01-type HIV-1 neutralizing antibodies in the VRC01-rearranging mouse model, although it did not achieve fully mature VRC01-class bnAbs (Tian et al., Cell, 2016).

Described herein are even more physiologically relevant mouse models for, e.g., testing candidate HIV-1 vaccine strategies and for disovering/optimizing humanized antibodies. A strategy related to that of the VRCO1 IgH chain rearranging model is utilized to engineer a mouse model that generates highly diverse IgL chain repertoires of potential VRC0101 precursors. When combined with the VRCO1 IgH rearranging model, the IgL rearranging model generates extremely diverse primary BCR repertoire of VRCO1 precursors in mouse for testing immunization strategies to elicit VRC01-class bnAb. A model is provided herein in which expression of bnAb affinity maturation intermediate is targeted specifically to mouse germinal center B cells. This approach, which expresses bnAb intermediates at a physiologically relevant stage, while avoiding potential central or peripheral tolerance checkpoints, is especially important for testing boost immunogens in sequential vaccination strategies.

Because these models can be produced via the rapid RAG-2 deficient blastocyst complementation (RDBC) technology, cohorts of mice can be produced more quickly than with conventional germline breeding.

Mouse models expressing bnAbs or their precursors are commonly used as assay systems to test and optimize immunogens at the preclinical stage (1). To generate such mouse models, one approach has been to integrate pre-rearranged V(D)J exons encoding the IgH or IgL variable regions of presumptive unmutated common ancestor (UCA) of bnAbs into the endogenous mouse JH or Jκ loci. Models made by this conventional “knock-in” approach has several limitations, including:
1) Because of allelic exclusion, the pre-rearranged V(D)J exons of bnAb UCA inhibit the rearrangement of endogenous mouse IgH and IgL loci (2). As a result, a unique human Ig heavy or light chain dominates the model mouse antibody repertoire (3-6), Thus, such models cannot evaluate the ability of immunogens to target antibody responses to relevant epitopes in complex antibody repertoires. The issue is particularly relevant to priming the development of HIV-1 bnAbs. Many of the most effective HIV-1 bnAbs exhibit one or more unusual characteristics (7), and B cells expressing the corresponding precursor antibodies are likely present at very low frequencies in human B cell compartments. Thus, an effective priming antigen in such models must be able to selectively engage the very rare B cells expressing bnAb precursor antibodies among an overwhelming majority of other B cells.
2) The CDR3 sequence of bnAb UCAs usually cannot be precisely defined, because CDR3 includes non-templated nucleotides introduced by terminal deoxynucleotydl transferase (TdT) during V(D)J recombination (2) and also can be mutated further by activation induced cytidine deaminase (AID) during antibody affinity maturation (8). Because of this ambiguity, the knock-in mouse models express usually germline-reverted version of bnAbs that are composed of germline V and J segments, but have CDR3s that may not represent those of the actual UCAs (3-6).
3) Certain bnAbs and their precursors are polyreactive or autoreactive, and B cells expressing them are usually eliminated or rendered anergic by tolerance control mechanisms either in the bone marrow or the periphery, or both in transgenic mouse models (9-11).
4) As expression of complete Ig genes begins prematurely at the pro-B cell stage in the knock-in transgenic mouse models, this method may not be suitable for expressing affinity maturation intermediates, which are generated during germinal center reactions in peripheral lymphoid tissues (12).

As an alternative to the knock-in mouse model approach, recent studies employed mice with fully human Ig variable region loci, such as Kymab mice (13), that can generate more complex primary antibody repertoires. However, as mice have far fewer B cells than humans, the actual antibody repertoire in such humanized mice is far smaller than the typical human counterpart. For this reason, the chance of finding a specific bnAb precursor in such Ig-humanized mice is substantially lower than in humans. Thus, when candidate immunogens are tested in these mice, it is difficult to interpret negative outcomes, which could be ascribed either to ineffectual immunogens or to the lack of B cells expressing the relevant antibody at the time of immunization.

To address the limitations of the mouse HIV-1 vaccine models discussed above, described herein is a new type of mouse vaccine model for the potent VRCO1 class of HIV-1 bnAbs, based on a strategy that allows the precursor human Immunoglobulin heavy (IgH) chain variable region exon for this bnAb to be developmentally assembled by V(D)J recombination and to dominate the IgH repertoire of the mice (6). In this VRC01-rearranging model, most individual B cells express one of a multitude of different variations of the potential VRCO1 precursor IgH chain, providing much more human-like precursor VRCO1 repertoire than other types of mouse models described above. Indeed, sequential immunization induced affinity maturation of VRC01-type HIV-1 neutralizing antibodies in the VRC01-rearranging mouse model, although it did not achieve fully mature VRC01-class bnAbs (6). Described herein is the development of an even more physiologically relevant mouse model for testing candidate HIV-1 vaccine strategies.

One model is based on the general strategy, which was employed for the VRCO1 IgH chain rearranging model, to engineer a mouse model that generates highly diverse IgL repertoires of VRCO1 precursor antibodies. When combined with the VRCO1 IgH rearranging model, the IgL rearranging model will generate extremely diverse primary human BCR repertoires of VRCO1 precursors in mice for testing immunization strategies to elicit VRC01-class bnAb.

The second model involves the targeting of human bnAb affinity maturation intermediates specifically to mouse germinal centers B cells. This approach, which will express bnAb intermediates at a physiologically relevant stage, while avoiding potential central or peripheral tolerance checkpoints, will be important for testing boost immunogens in sequential vaccination strategies.

Finally, these models can be generated with RAG-2 deficient blastocyst complementation technology (14), which obviates the lengthy and costly process of germline breeding and which permits supply of mouse models in a timely manner.

### REFERENCES

1. L. Verkoczy, F. W. Alt, M. Tian, Human Ig knockin mice to study the development and regulation of HIV1 broadly neutralizing antibodies. Immunological reviews 275, 89-107 (2017).
2. F. W. Alt, Y. Zhang, F. L. Meng, C. Guo, B. Schwer, Mechanisms of programmed DNA lesions and genomic instability in the immune system. Cell 152, 417-429 (2013).
3. J. G. Jardine et al., HIV-1 VACCINES. Priming a broadly neutralizing antibody response to HIV-1 using a germline-targeting immunogen. Science (New York, N.Y.) 349, 156-161 (2015).
4. P. Dosenovic et al., Immunization for HIV-1 Broadly Neutralizing Antibodies in Human Ig Knockin Mice. Cell 161, 1505-1515 (2015).
5. A. T. McGuire et al., Specifically modified Env immunogens activate B-cell precursors of broadly neutralizing HIV-1 antibodies in transgenic mice. Nat Commun 7, 10618 (2016).
6. M. Tian et al., Induction of HIV Neutralizing Antibody Lineages in Mice with Diverse Precursor Repertoires. Cell 166, 1471-1484.e1418 (2016).
7. D. R. Burton, L. Hangartner, Broadly Neutralizing Antibodies to HIV and Their Role in Vaccine Design. Annu Rev Immunol 34, 635-659 (2016).
8. J. M. Di Noia, M. S. Neuberger, Molecular mechanisms of antibody somatic hypermutation. Annu Rev Biochem 76, 1-22 (2007).
9. L. Verkoczy et al., Autoreactivity in an HIV-1 broadly reactive neutralizing antibody variable region heavy chain induces immunologic tolerance. Proceedings of the National Academy of Sciences of the United States of America 107, 181-186 (2010).
10. C. Doyle-Cooper et al., Immune tolerance negatively regulates B cells in knock-in mice expressing broadly neutralizing HIV antibody 4E10. Journal of immunology (Baltimore, Md.: 1950) 191, 3186-3191 (2013).
11. Y. Chen et al., Common tolerance mechanisms, but distinct cross-reactivities associated with gp41 and lipids, limit production of HIV-1 broad neutralizing antibodies 2F5 and 4E10. Journal of immunology (Baltimore, Md.: 1950) 191, 1260-1275 (2013).
12, G. D. Victora, M. C. Nussenzweig, Germinal centers. Annu Rev Immunol 30, 429-457 (2012).
13. D. Sok et al., Priming HIV-1 broadly neutralizing antibody precursors in human Ig loci transgenic mice. Science (New York, N.Y.) 353, 1557-1560 (2016).
14. J. Chen, R. Lansford, V. Stewart, F. Young, F. W. Alt, RAG-2-deficient blastocyst complementation: an assay of gene function in lymphocyte development. Proceedings of the National Academy of Sciences of the United States of America 90, 4528-4532 (1993).

### Example 6

Current vaccine strategies to elicit broadly neutralizing antibodies (bnAbs) against HIV-1 are based on sequential immunizations with separate immunogens that target B cells expressing precursor and intermediate forms of bnAbs, respectively (1-5). Described herein are novel and effective mouse models to test and optimize such sequential immunization protocols for eliciting the potent VRCO-1 class of HIV-1 bnAbs (6-9).

Each immunoglobulin heavy (IgH) or light (IgL) chain variable region contains three complementarity determining regions (CDRs) that are particularly important for antigen contact (10). CDR1 and CDR2 are encoded in each germline variable region segment (V) and are unique to each of the multiple germline IgH and IgL V segments. CDR3 is assembled at the junction of IgH V, D, and J segments or IgL V and J segments, in association with non-templated de novo junctional diversification mechanisms such as N region additions by TdT (11, 12). For this reason, CDR3 represents by far the most diverse portion of antibodies.

VRC01-class bnAbs target the CD4-binding site of HIV-1 Envelop (Env) protein and use exclusively the human IgH VH1-2 segment (6-9). In this regard, the germline VH1-2 encodes sequences that allow it to mimic CD4 interaction with gp120. In this unusual mode of antigen interaction, the VH1-2 accounts for nearly 60% of the interface of VRCO1 bnAbs with gp120 (7). In contrast, interactions of most other types of HIV-1 bnAbs with Env epitopes rely heavily on a unique, and in many cases exceptionally long, IgH chain CDR3s (CDR H3) (13).

In this regard, while VH1-2-based Ig heavy chains are quite common in human antibodies (14, 15), only a small number of individuals may harbor antibodies with the unusual de novo generated CDR H3 found in these other types of HIV-1 bnAbs. Thus, elicitation of VRCO1 class antibodies may be more probable in human populations than elicitation of other types of bnAbs. However, VRCO1 antibodies also require Ig K light chains with an unusually short 5-amino acid CDR L3 (6-9). Moreover, three VK segments (VK3-20, Vk3-11 and Vk1-33) are primarily involved in coding for VRCO1 Ig light chains, apparently because the short CDR L1 of these Vk segments can more easily accommodate glycans that shield the CD4 binding site. CDR H3, although not strictly conserved, also influences the function of VRCO1 antibodies (16).

The various restrictions outlined above reduce the pool of potential VRC01-like precursors to just a small subset of total human antibodies that employ VH1-2. Indeed, the frequency of human B cells expressing VRC01-like precursor antibodies was estimated to be about 1 in 2.4 million (17). Adding to the difficulty in their elicitation via immunization strategies, mature VRC01-class bnAbs exhibit a massive level (up to 40% of nucleotides) of somatic hypermutations, some of which are required for neutralization breadth and potency (6-9, 18). To elicit VRC01-class bnAbs via sequential immunization, priming immunogens have been designed to selectively activate the rare B cells expressing potential VRC01-like precursor antibodies (3, 4, 19). Following priming, a series of boost immunogens has been designed to gradually mature the precursor antibodies, through intermediate stages, and onward toward the high mutated mature VRC01-class bnAbs (20, 21). To facilitate the testing of such complex immunization strategies, we recently developed a new type of mouse vaccine model for VRC01-class bnAbs, based on a strategy that allows the precursor human IgH variable region exon for this bnAb to be developmentally assembled by V(D)J recombination and to dominate the mouse IgH repertoire (21). In this “VRCO1-IgH rearranging” model, most individual B cells, due to de novo CDR H3 diversification, express one of a multitude of different variations of the potential VRCO1 precursor IgH chain, providing much more human-like precursor VRCO1 repertoire than conventional transgenic mice that express a pre-rearranged germline-reverted VRCO1 IgH chain. Indeed, sequential immunization of this VRCO1 IgH rearranging mouse model induced affinity maturation of VRC01-type HIV-1 neutralizing antibodies, although it did not achieve fully mature VRC01-class bnAbs (21).

Described herein are two aims that are focused on developing two types of even more physiologically relevant mouse models for testing candidate HIV-1 vaccine strategies to elicit VRC01-class bnAbs. In a third aim, described is the use of the RAG-2 deficient blastocyst complementation (RDBC) approach (22) to rapidly generate cohorts of the existing VRCO1 model and new models.

Aim 1. Generation of VCR01 mouse models with diverse bnAb IgH and IgL precursor repertoires.

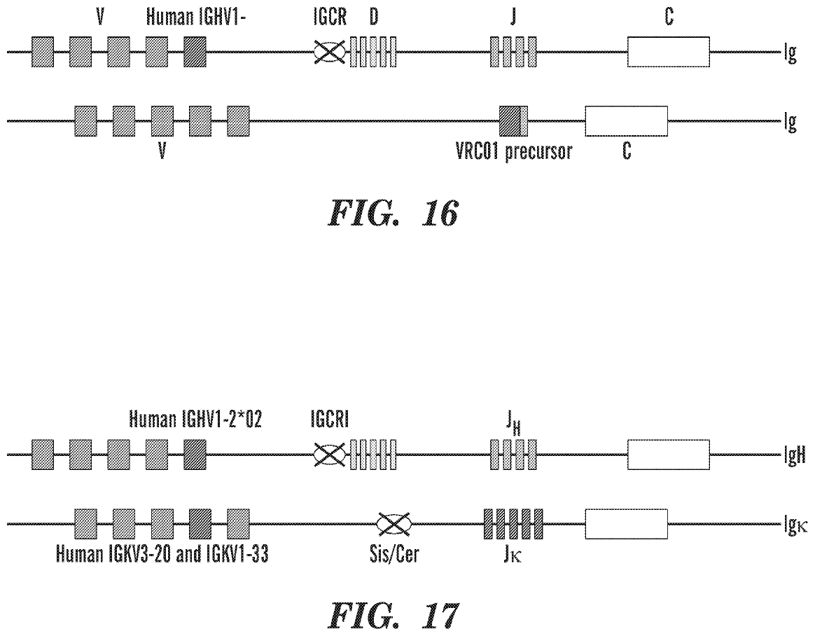

Our design for the prior VRC01 vaccine mouse mdoel was based primarily on the finding that rearrangement of the most D-proximal mouse VH gene segment (CH81x) is under the control of a regulatory element referred to herein to as intergenic control region 1(IGCR1)(23). When IGCR1 is inactivated, VH81X is used in most VH to DJH joining events, despite integrity of the remaining IgH locus (23, 24). Thus, when human VH1-2 was substituted for mouse VH81X and deleted IGCR1 on the same IgH allele in mice (FIG. 16), VH1-2 was highly represented in the primary IgH repertoires of mature B cells (21). Furthermore, because VH1-2 underwent V(D)J recombination and was, subject to normal junctional diversification mechanisms, it was expressed in association with an extremely diverse range of CDR H3s (21).

In immunization experiments with the VH1-2 rearranging model, affinity maturation of the VH1-2-based Ig heavy chain, which accounts for the bulk of antigen contact, was focused on. For this reason, described herein is a model that expressed a pre-rearranged version of the germline-reverted VRCO1 Ig Vκ 3-20 light chain (FIG. 16), which was expressed in 94% of mature B cells (21). Described herein is the generation of a mouse VRC01-rearranging model that expresses diverse VRCO1 precursors for both Ig light and heavy chains. For example, human Vx3-20 and Vx1-33, the two most commonly used Ig light chain segments among VRCO1 antibodies (6, 8, 9), can be utilized in de novo rearrangement in developing precursor B cells in mice. The strategy to accomplish this goal is based on the finding that suppression of dominant V(D)J recombination of proximal IgL Vκ segments is also mediated by a V(D)J recombination regulatory element, termed Sis/Cer, that functions analogously to IGCR1 in the IgH locus (FIG. 17) (25). Thus, when the Sis/Cer elements is deleted in Igk locus, the several most proximal Vk gene segments dominate the Vκ to Jκ rearrangement process. Based on this finding, the human Vk3-20/Vk1-33 segments can be positioned at the proximal end of Vκ cluster relative to Jκ segments in the context of a Cer/Sis deletion (FIG. 17). This VRC01 light chain rearrangement system can be combined with the VH1-2-rearranging model to generate a mouse model that produces diverse VH1-2 heavy chains and diverse Vk3-20/Vk1-33 Igk light chains. This mouse model serves as an even more physiologically relevant system to test candidate vaccine strategies than our prior VRCO1 models. This model can also lower the frequencies of VH1-2 heavy chains and/or Vk3-20/Vk1-33 light chains by retaining IGCR1 and/or Cis/Ser in the model to test immunization protocols in a more stringent manner.

The mouse Igκ repertoire shows relatively limited junctional diversity (e.g. N regions) compared to that of IgH, potentially due to lack of TdT expression in mouse pre-B cells in which Igk rearrangement occurs (26,27). In this regard, it has also been shown that certain dendritic T cells subsets that develop in the absence of TdT form repetitive (“canonical”) V(D)J junctions mediated by local micro-homologies (28). In contrast, data provided herein (and elsewhere) indicate that the human Igk repertoire exhibits evidence of substantial junctional diversification in CDR3, confirming prior observations made with a more limited data set (29).

It is contemplated herein that TdT expression in human pre-B cells may be responsible for increased CDR3 junctional diversification of the human Ig light chain repertoire than that of the mouse counterpart. Consistent with this hypothesis, constitutive expression of TdT throughout B cell development in a transgenic mouse led to evident N-nucleotide addition in CDR3 of mouse Ig light chains (30). To further humanize the Ig light chain repertoire in the VRCO1 mouse model, a TdT transgene driven by CD19 promoter can be introduced to the VRCO1 Igk rearranging mouse model. HTGTS-rep-seq assay can assess Igk CDR3 junctions in the Igx-rearranging model with or without enforced TdT expression and the levels and types of junctional diversifications compared to those found in human Igx repertoires. If enforced TdT expression does indeed generate a more human-like diverse Igk repertoire, this component will be built in as a feature of humanized Igk rearranging VRCO1 model to permit the mouse model to generate a Igk repertoire more representative of that of human B cells.

Prior VRCO1 models either rearrange mouse IgL chains or have a knock-in pre-rearranged human germline-reverted (gl) VRCO1 light chain. The fixed gl-VRCO1 light chain facilitates the initial testing of immunization strategies, but does not represent a physiological setting. On the other hand, the model without the gl-VRCO1 light chain relies on mouse Ig light chains, in association with the human VH1-2 heavy chain, to reconstitute VRC01-like antibodies. Although mouse Ig light chain rearrangements can also generate the signature 5-amino acid CDR L3s, other aspects of the human Ig light chain may also be important for the function of VRC01-class antibodies. Presumably for this reason, most VRC01-class bnAbs use human Vk3-20 and Vk1-33, which lack close mouse homologues.

These concerns are addressed herein by expressing diverse repertoires of both VH1-2 heavy chain and Vk3-20 and Vk1-33 Ig light chains. Thus, the new model represents a substantial improvement over prior models. As discussed above, this new model should also be superior to Kymice or similar Ig-humanized mice, because it is expected to contain a much higher frequencies of B cells expressing relevant IgH and IgL chains and, thus, a more “human-like” primary repertoire for the VRCO1 lineage. Also incorporated is enforced TdT expression to generate a more human-like diverse IgL chain CDR3 primary repertoire

In the design of this new IgH and IgL rearranging VRC01 mouse model, endogenous mouse D and J segments are employed. In this regard, the mouse JHs are very homologous to human JHs. For the VRCO1 lineage of antibodies, the human JH2 provides a key tryptophan residue to CDR H3 (16, 31). Mouse JH1 is homologous to human JH2 and contains the analogous tryptophan residue. Indeed, when the VRCO1 mouse model with mouse JHs was immunized with immunogens designed to elicit VRC01-like antibodies, all the HIV-1 neutralizing antibodies utilized mouse JH1 and contained the signature tryptophan residue in CDR H3 (21). This result indicates that a model with mouse JHs heads us down the right path.

To further increase the frequency of VRC01-like precursors in the model, there is provided herein a mouse line that incorporates both human JH2 and VH1-2. (Alt lab, unpublished results).

It is hard to ascertain the identities of the germline D segments in VRC01-class antibodies, because the CDR H3 region is subject to both junctional diversification and extensive somatic hypermutation. Other than the tryptophan residue mentioned above, no other conserved features are discernable in the CDR H3 region of VRCO1 family members. It is possible that precursor antibodies with a variety of CDR H3s may potentially evolve into VRC01-like antibodies. In conjunction with junctional diversification, mouse Ds are expected to contribute similar levels of diversity to CDR H3 region as human Ds, and should create a large repertoire of VRC01-like precursor that would serve as relevant targets for immunogens. There is no strong conservation of JK usage among VRCO1 lineage antibodies. In addition, mouse Jks are almost identical to human Jks. However, human Jks could be easily added to the model if desired.

Aim 2. Mouse Models that Express bnAb Intermediates Directly in Germinal Center B Cells

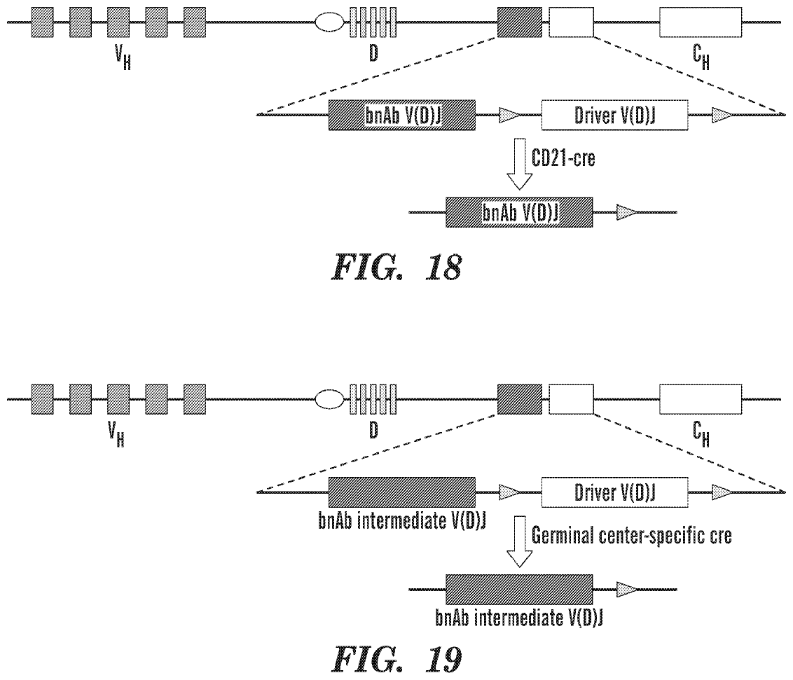

B cells expressing certain bnAbs or their precursors tend to be deleted during B cell maturation in mice (32). To overcome this hurdle, we developed a conditional expression approach that confines bnAb expression to mature B cells, thereby circumventing the hurdle of tolerance control in bone marrow. In this approach, B cell maturation is driven by innocuous Ig heavy and Ig light chain variable region exons, which are termed “driver Ig genes” (FIG. 18). The driver Ig genes are flanked by loxP sites and are deleted by CD21-cre, which is expressed specifically at the mature B cell stage (33). The bnAb IgH V(D)J exon is positioned upstream of driver Ig V(D)J exon and is expressed in mature B cells after the deletion of driver Ig V(D)J exon by CD21-cre. This conditional expression strategy can bypass tolerance control mechanisms that impede the expression of VRC26 precursor, an antibody with extraordinarily long CDR H3 (34). We have also established an analogous system for the conditional expression of bnAb Ig light chains, and achieved conditional expression of both the Ig heavy and Ig light chains for the UCA of DH270 bnAb(35) (data not shown).

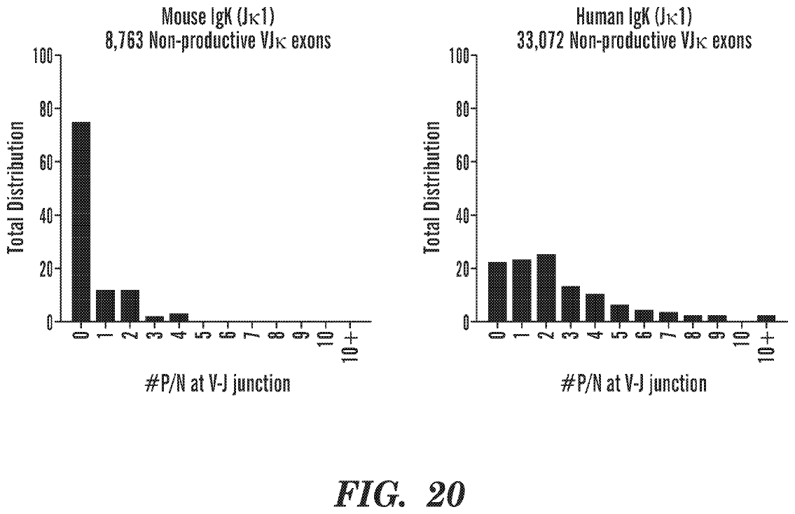

It is contemplated herein that this conditional expression technology can be adapted to express both the Ig heavy and Ig light chains of affinity maturation intermediates of bnAbs by employing a conditional expression cassette in which cre expression is driven under the control of a germinal center-specific promoter (FIG. 19). To optimize this approach, the effectiveness of cre transgene driven by the S1pr2 promoter can be compared to the Cγ1-promoter, as both promoters have been used to enforce germinal center B cell specific expression of cre (36, 37). Alternative GC-specific or GC-biased promoters can be used. For this conditional approach, driver V exons must not only support B cell development in the bone marrow, but must also promote the activation of B cells in the context of the germinal center reaction. Thus, driver V exons must encode an antibody with known antigen-binding specificity so that immunization with this target antigen will promote germinal center reactions. The driver IgH V exon in our current tested conditional expression cassette encodes an antibody that recognizes the HA antigen of influenza (38).

Thus, immunization with HA antigen should induce germinal center reactions, during which deletion of the driver V gene will lead to the expression of the V(D)J exon encoding VRCO1 intermediate target antibodies. The survival and maturation of the nascent germinal center B cells that express bnAb intermediates will depend on antigens that can interact with their BCR. Thus, in some embodiments, the boost immunogen can be administered together with the HA antigen so that it will be available to stimulate affinity maturation in germinal center B cells that have switched on the expression of a given bnAb intermediate target antibody. Besides HA, antibodies against NP (B1-8) can be used as the driver, and in this case, immunization with NP will induce GC reaction and the expression of target antibody in GC B cells.

An alternative possibility will be to use the germline VRCO1 antibody as the driver V gene, in which case the mice will be immunized with a mixture of prime and boost immunogens. The prime immunogen will initiate germinal reactions and activate the expression of intermediate antibody. Then, the stage would be set for testing the boost immunogen. After this round of boost immunization, the memory B cells from the germinal center reaction will serve as targets for further boost immunizations.

The germinal center-specific expression model permits evaluation of boost immunogens in several respects. For example, it can be tested whether the immunogen can effectively promote somatic hypermutation of bnAb intermediates, recruit T follicular helper cells (Tfh) to the germinal center reaction, and favor memory B cell development over terminal differentiation to plasma cells. If bnAb maturation is accompanied by the acquisition of poly-reactivity or auto-reactivity, the model would also provide an opportunity to study the fate of such affinity maturation intermediates in germinal centers. The evolution of UCA to mature bnAb will involve many intermediates. For initial studies, the most potent VRC01-like neutralizing antibody isolated from our previous immunization experiments (21) can be used as the intermediate antibody in the system. Further intermediate antibodies of interest can be incorporated as desired.

There have been several examples where tolerance control mechanisms hinder the expression of bnAbs or their precursors, as shown in mouse models for 2F5, 4E10 (MPER bnAb) (39-41) or 3BNC60 (CD4 binding site bnAb) (42) and our own unpublished data on mouse models for DH270 (V3glycan bnAb) (35) and VRC26 (V1V2 bnAb) (34). Given these precedents, it is possible that expressing affinity maturation VRC01bnAB-lineage intermediates, or other desired antibodies we would seek to optimize via the conventional transgenic knock-in approach may run into similar roadblocks. Moreover, central and peripheral tolerance control mechanisms normally target precursor and naïve B cells, respectively. Since affinity maturation intermediates arise from GC reactions, they would not be subject to these checkpoints under physiological conditions. Thus, expressing intermediates with the conventional knockin strategy essentially imposes non-physiological restrictions on these antibodies. The present GC-specific expression strategy is specifically designed to address this issue. With the conventional knock-in approach, intermediate antibodies are expressed in naïve B cells. In contrast, in a normal immunization setting, memory B cells expressing intermediate antibodies are the physiological targets of boost immunization. Since naïve B cells and memory B cells can differ in their immune response, constitutive expression models of intermediate antibodies may not provide accurate assessments of boost immunogens. By expressing intermediate antibodies in germinal center B cells, a subset of which can differentiate into memory B cells, the most relevant setting is recreated for boost immunization.

### Aim 3. Provision of Cohorts of VRCO1 Mouse Models

To ensure an efficient supply of the existing and new mouse models for these immunization experiments, the Rag2-deficient complement (RDBC) system can be used to generate the mouse models in the context of chimeric mice (22). In this approach, the genetic modifications are introduced into ES cells which is injected into Rag2-deficient blastocysts to generate chimeric mice. Because Rag2 is essential for V(D)J recombination, all the B and T cells in the RDBC chimeras are derive from the injected ES cells. As already shown, such chimeric mice can be used directly for immunization experiments (21). The RDBC approach obviates the need for lengthy and costly breeding involved in conventional germline transmission; the advantages of this approach is especially obvious in the context of eliminating years of breeding to generate mouse models involving multiple genetic modifications, such as those proposed herein. As with the initial VRC01 model, the chimeras will also be bred for germline transmission Summary and Discussion.

Described herein are two types of mouse models that facilitate the development of sequential immunization approaches for the generation of HIV-1 vaccines. The rearrangement model described in Aim 1 can be used to test both priming and boosting steps of the immunization protocol, whereas the germinal center-specific model in Aim 2 would specifically aid in studying boost immunizations, including testing strategies to circumvent potential roadblocks that may be incurred.

Relative to Kymab mice, our proposed mouse Aim 1 mouse model, which expresses VH1-2 and Vk3-20, Vk1-33 through rearrangements, is designed to have higher frequencies of VRC01-like precursors. We can use appropriate probes, for example eOD-GT8, to assess the frequency of VRC01-like precursors (17). If the mouse contains readily detectable precursors, but does not respond to test immunogens, the result would suggest that the immunogen is not acting as effective activator of target B cells. The advantage of these proposed methods, especially the scheme to express intermediate antibodies specifically at the germinal center stage, will be important for testing boost immunogens. In a conventional mouse model, negative results in boost immunization could have at least two potential interpretations. One possibility is that the previous immunization, for instance the priming step, failed to elicit the relevant intermediate antibody targeted by the boost immunogen. Alternatively, the B cells expressing intermediate antibody may have been generated, but did not respond to the boost immunogen. These two potential possibilities would point to different directions for the next steps.

The Aim 2 model can eliminate these potentially confounding ambiguities by producing a population of germinal center B cells expressing a defined affinity maturation intermediate. In this model, lack of response in boost immunizations can be firmly ascribed to ineffectual boost immunization. If a novel priming immunogen eventually works more effectively in Kymab mice or similar mouse models than eOD-GT8, the paucity of VRC01-like precursors in these mice likely may still pose a formidable challenge in the boosting step, as discussed above.

These models, with higher frequencies of relevant vaccine targets and/or more appropriate expression patterns, offer a more tractable system for immunization studies. If priming immunization with eOD-GT8 does elicit VRC01-like antibodies in humans during clinical trials, the next major challenge is to devise boost immunization strategies to mature the intermediate antibodies further toward bnAbs. Like the development of priming immunogens, such as eOD-GT8, the optimization of boost immunogens would also require iterative experimentation in animal models, and the proposed mouse models would be well suited for this purpose. The proposed strategies, either the rearrangement model or GC-specific expression model, permit mouse models expressing intermediate VRC01-like antibodies identified in clinical trials, and these mouse models can be used to test boost immunogens for the next steps.

### REFERENCES

1. D. S. Dimitrov, Therapeutic antibodies, vaccines and antibodyomes. MAbs 2, 347-356 (2010).
2. B. F. Haynes, G. Kelsoe, S. C. Harrison, T. B. Kepler, B-cell-lineage immunogen design in vaccine development with HIV-1 as a case study. Nat Biotechnol 30, 423-433 (2012).
3. J. Jardine et al., Rational HIV immunogen design to target specific germline B cell receptors. Science (New York, N.Y.) 340, 711-716 (2013).
4. A. T. McGuire et aL, Engineering HIV envelope protein to activate germline B cell receptors of broadly neutralizing anti-CD4 binding site antibodies. The Journal of experimental medicine 210, 655-663 (2013).
5. X. Xiao et al., Germline-like predecessors of broadly neutralizing antibodies lack measurable binding to HIV-1 envelope glycoproteins: implications for evasion of immune responses and design of vaccine immunogens. Biochem Biophys Res Commun 390, 404-409 (2009).
6. X. Wu et al., Rational design of envelope identifies broadly neutralizing human monoclonal antibodies to HIV-1. Science (New York, N.Y.) 329, 856-861 (2010).
7. T. Zhou et al., Structural basis for broad and potent neutralization of HIV-1 by antibody VRC01. Science (New York, N.Y.) 329, 811-817 (2010).
8. J. F. Scheid etal., Sequence and structural convergence of broad and potent HIV antibodies that mimic CD4 binding. Science (New York, N.Y.) 333, 1633-1637 (2011).
9. X. Wu et al., Focused evolution of HIV-1 neutralizing antibodies revealed by structures and deep sequencing. Science (New York, N.Y.) 333, 1593-1602 (2011).
10. F. W. Alt, Y. Zhang, F. L. Meng, C. Guo, B. Schwer, Mechanisms of programmed DNA lesions and genomic instability in the immune system. Cell 152, 417-429 (2013).
11. F. W. Alt, D. Baltimore, Joining of immunoglobulin heavy chain gene segments: implications from a chromosome with evidence of three D-JH fusions. Proceedings of the National Academy of Sciences of the United States of America 79, 4118-4122 (1982).
12. T. Komori, A. Okada, V. Stewart, F. W. Alt, Lack of N regions in antigen receptor variable region genes of TdT-deficient lymphocytes. Science (New York, N.Y.) 261, 1171-1175 (1993).
13. D. R. Burton, L. Hangartner, Broadly Neutralizing Antibodies to HIV and Their Role in Vaccine Design. Annu Rev Immunol 34, 635-659 (2016).
14. B. J. DeKosky et al., High-throughput sequencing of the paired human immunoglobulin heavy and light chain repertoire. Nat Biotechnol 31, 166-169 (2013).
15. S. G. Lin et al., Highly sensitive and unbiased approach for elucidating antibody repertoires. Proceedings of the National Academy of Sciences of the United States of America, (2016).
16. C. Yacoob et al., Differences in Allelic Frequency and CDRH3 Region Limit the Engagement of HIV Env Immunogens by Putative VRCO1 Neutralizing Antibody Precursors. Cell reports 17, 1560-1570 (2016).
17. J. G. Jardine et al., HIV-1 broadly neutralizing antibody precursor B cells revealed by germline-targeting immunogen. Science (New York, N.Y.) 351, 1458-1463 (2016).
18. F. Klein et al., Somatic mutations of the immunoglobulin framework are generally required for broad and potent HIV-1 neutralization. Cell 153, 126-138 (2013).
19. L. Stamatatos, M. Pancera, A. T. McGuire, Germline-targeting immunogens. Immunological reviews 275, 203-216 (2017).
20. B. Briney et al, Tailored Immunogens Direct Affinity Maturation toward HIV Neutralizing Antibodies. Cell 166, 1459-1470.e1411 (2016).
21. M. Tian et al., Induction of HIV Neutralizing Antibody Lineages in Mice with Diverse Precursor Repertoires. Cell 166, 1471-1484.e1418 (2016).
22. J. Chen, R. Lansford, V. Stewart, F. Young, F. W. Alt, RAG-2-deficient blastocyst complementation: an assay of gene function in lymphocyte development. Proceedings of the National Academy of Sciences of the United States of America 90, 4528-4532 (1993).
23. C. Guo et al., CTCF-binding elements mediate control of V(D)J recombination. Nature 477, 424-430 (2011).
24. J. Hu et aL, Chromosomal Loop Domains Direct the Recombination of Antigen Receptor Genes. Cell 163, 947-959 (2015).
25. Y. Xiang, S. K. Park, W. T. Garrard, A major deletion in the Vkappa-Jkappa intervening region results in hyperelevated transcription of proximal Vkappa genes and a severely restricted repertoire. Journal of immunology (Baltimore, Md. 1950) 193, 3746-3754 (2014).
26. Y. S. Li, K. Hayakawa, R. R. Hardy, The regulated expression of B lineage associated genes during B cell differentiation in bone marrow and fetal liver. The Journal of experimental medicine 178, 951-960 (1993).
27. K. D. Victor, K. Vu, A. J. Feeney, Limited junctional diversity in kappa light chains. Junctional sequences from CD43+B220+ early B cell progenitors resemble those from peripheral B cells. Journal of immunology (Baltimore, Md.: 1950) 152, 3467-3475 (1994).
28. Y. Zhang et al., The role of short homology repeats and TdT in generation of the invariant gamma delta antigen receptor repertoire in the fetal thymus. Immunity 3, 439-447 (1995).
29. H. J. Girschick, P. E. Lipsky, The kappa gene repertoire of human neonatal B cells. Molecular immunology 38, 1113-1127 (2002).
30. L. A. Bentolila et al., Constitutive expression of terminal deoxynucleotidyl transferase in transgenic mice is sufficient for N region diversity to occur at any Ig locus throughout B cell differentiation. Journal of immunology (Baltimore, Md.: 1950) 158, 715-723 (1997).
31. A. P. West, Jr., R. Diskin, M. C. Nussenzweig, P. J. Bjorkman, Structural basis for germ-line gene usage of a potent class of antibodies targeting the CD4-binding site of HIV-1 gp120. Proceedings of the National Academy of Sciences of the United States of America 109, E2083-2090 (2012).
32. L. Verkoczy, F. W. Alt, M. Tian, Human Ig knockin mice to study the development and regulation of HIV1 broadly neutralizing antibodies. Immunological reviews 275, 89-107 (2017).
33. M. Kraus, M. B. Alimzhanov, N. Rajewsky, K. Rajewsky, Survival of resting mature B lymphocytes depends on BCR signaling via the lgalpha/beta heterodimer. Cell 117, 787-800 (2004).
34. N. A. Doria-Rose et al., Developmental pathway for potent VIV2-directed HIV-neutralizing antibodies. Nature 509, 55-62 (2014).
35. M. Bonsignori et aL, Staged induction of HIV-1 glycan-dependent broadly neutralizing antibodies. Science translational medicine 9, (2017).
36. R. Shinnakasu et aL, Regulated selection of germinal-center cells into the memory B cell compartment. Nature immunology 17, 861-869 (2016).
37. S. Sander et al., Synergy between PI3K signaling and MYC in Burkitt lymphomagenesis. Cancer cell 22, 167-179 (2012).
38. J. Kavaler, A. J. Caton, L. M. Staudt, D. Schwartz, W. Gerhard, A set of closely related antibodies dominates the primary antibody response to the antigenic site CB of the A/PR/8/34 influenza virus hemagglutinin. Journal of immunology (Baltimore, Md.: 1950) 145, 2312-2321 (1990).
39. L. Verkoczy et al., Autoreactivity in an HIV-1 broadly reactive neutralizing antibody variable region heavy chain induces immunologic tolerance. Proceedings of the National Academy of Sciences of the United States of America 107, 181-186 (2010).
40. Y. Chen et al., Common tolerance mechanisms, but distinct cross-reactivities associated with gp41 and lipids, limit production of HIV-1 broad neutralizing antibodies 2F5 and 4E10. Journal of immunology (Baltimore, Md.: 1950) 191, 1260-1275 (2013).
41. C. Doyle-Cooper et al., Immune tolerance negatively regulates B cells in knock-in mice expressing broadly neutralizing HIV antibody 4E10. Journal of immunology (Baltimore, Md., 1950) 191, 3186-3191 (2013).
42. A. T. McGuire et al., Specifically modified Env immunogens activate B-cell precursors of broadly neutralizing HIV-1 antibodies in transgenic mice. Nat Commun 7, 10618 (2016).
43. R. W. Sanders, J. P. Moore, Native-like Env trimers as a platform for HIV-1 vaccine design. Immunological reviews 275, 161-182 (2017).

### Aim 1

We have employed a Cas9-gRNA based approach to dete the Sis/Cer elements of the Igk locus in a mouse v-Abl pre-B cell line that we can induce in vitro to undergo Igx V(D)J recombination. After control and Sis/Cer deleted v-Abl pre-B cells were induced to undergo V(D)J recombination of their endogenous Igκ locus, HTGTS-based high throughput V(D)J recombination assay (3, 4) was used to analyze the frequency with which different endogenous VK segments rearranged to a Jk4 bait sequence, This study clearly demonstrates that deletion of Sis/Cer element substantially increased the rearrangement frequency of the proximal Vx3-1, Vx3-2 and Vx3-3 segment (FIG. 15A-15V). Given these observations, it is anticipated that human VK3-20 and Vk1-33 segments, when positioned in place of the proximal mouse VK segments in the context of Sis/Cer deletion, will also be preferentially utilized during V(D)J recombination. Due to junctional diversification, the B cell population in this model will be expected to express diverse repertoires of VK3-20 and Vk1-33 light chains; and, as described above, it can be tested whether such diversity may be made even more human-like by incorporation of constitutive TdT expression in the ES cell based model.

To further address whether Human VKJK repertoires might show increased junctional diversity versus those of mouse VkJk repertoires, HTGTS-Rep-seq analysis (4) was performed on DNA from WT mouse IgM+ splenic B cells and human peripheral blood mononuclear cells (PBMCs) using a mouse or human JK1 bait as a primer. To obviate the possibility of influences of cellular selection, presented are results of outof-frame (non-productive) WJK junctions. This preliminary analysis, while limited to just one human sample, shows a markedly greater incorporation of P and/or N junctional elements into the human VKJK junctions versus the mouse VKJK junctions (FIG. 20). These findings, which will be confirmed and extended by analysis of additional human samples, provide strong support for the goal of incorporating enforced TdT expression into the new Igx-rearranging VRCO1 model to allow it to generate a more human-like IgK repertoire.

### Aim 2

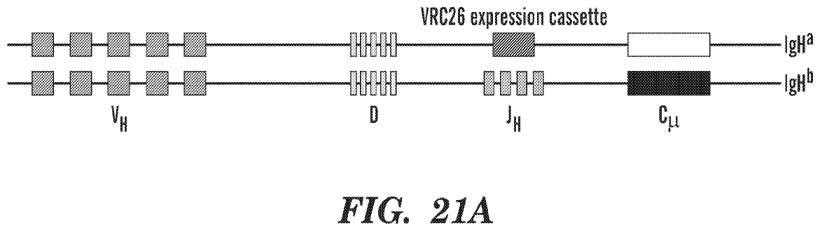

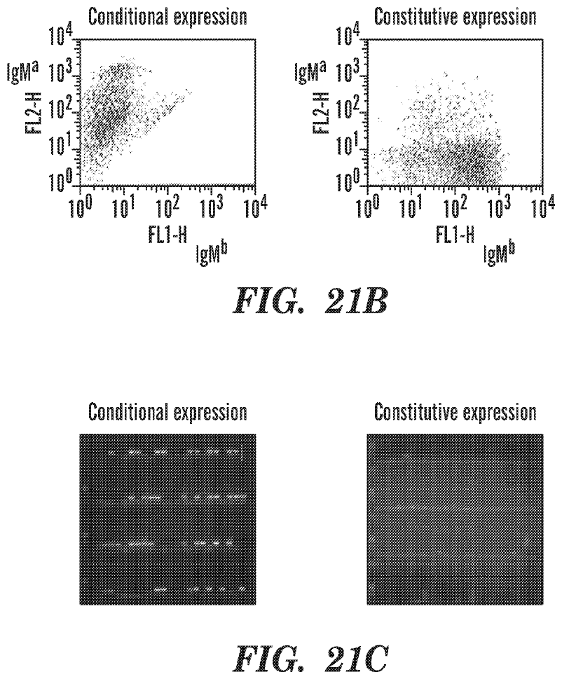

The conditional expression strategy has been employed to generate a VRC26UCA mouse model that activates expression of the VRC26UCA in peripheral B cells (FIG. 21A; Tian and Alt, unpublished). When the VRC26UCA heavy chain was expressed constitutively during B cell maturation, most B cells expressing VRC26UCA heavy chain were deleted in the bone marrow and, based on surface IgM expression, did not appear in the peripheral B cell compartment (FIG. 21B, 21C, right panel). In contrast, when VRC26UCA heavy chain was expressed conditionally in mature B cells, approximately 50% B splenic B cells expressed the knock-in VRC26UCA heavy chain on their surface (FIGS. 21B, 21C, left panel).

In addition to the VRCO1 and VRC26 mouse models described above, we have also generated, or are in the process of generating, multiple mouse models for other types of bnAbs against HIV-1 and influenza virus. For example, we have generated mouse models expressing two types of UCAs for DH270, which targets the V3 glycan epitope of HIV-1 Env (5). We also have generated a mouse model expressing the Ig heavy chain of DH511UCA, which recognizes the Membrane External Proximal Region (MPER) (6), and we are completing the model by incorporating the DH511UCA light chain. In addition, we are building a mouse model for CH235UCA, which targets the CD4-binding site in a similar manner as VRC01, but utilizes VH1-46 gene segment instead of VH1-2 (7). We are producing a mouse model for the 56.a.09 bnAb that targets the stem region of influenza HA antigen (8).

### REFERENCES

1. M. Tian et al., Induction of HIV Neutralizing Antibody Lineages in Mice with Diverse Precursor Repertoires. Cell 166, 1471-1484.e1418 (2016).
2. Y. Xiang, S. K. Park, W. T. Garrard, A major deletion in the Vkappa-Jkappa intervening region results in hyperelevated transcription of proximal Vkappa genes and a severely restricted repertoire. Journal of immunology (Baltimore, Md.: 1950) 193, 3746-3754 (2014).
3. J. Hu et al., Chromosomal Loop Domains Direct the Recombination of Antigen Receptor Genes. Cell 163, 947-959 (2015).
4. S. G. Lin et al., Highly sensitive and unbiased approach for elucidating antibody repertoires. Proceedings of the National Academy of Sciences of the United States of America, (2016).
5. M. Bonsignori et al., Staged induction of HIV-1 glycan-dependent broadly neutralizing antibodies. Science translational medicine 9, (2017).
6. LaTonya D. Williams et al., Potent and broad HIV-neutralizing antibodies in memory B cells and plasma. Sci. Immunol 2, (2017).
7. M. Bonsignori et aL, Maturation Pathway from Germline to Broad HIV-1 Neutralizer of a CD4-Mimic Antibody. Cell 165, 449-463 (2016).
8. M. G. Joyce et al., Vaccine-Induced Antibodies that Neutralize Group 1 and Group 2 Influenza A Viruses. Cell 166, 609-623 (2016).

What is claimed herein is:

1. A cell comprising at least one of:
a) an engineered IgH locus comprising a CBE element within the nucleic acid sequence separating the 3′ end of a target V_{H} segment and the 5′ end of the first V_{H} segment which is 3′ of the target V_{H} segment; and/or
b) an engineered IgL locus comprising at least one of:
i) a non-functional Cer/Sis sequence within the nucleic acid sequence separating the 3′ end of the 3′-most V_{L} segment and the 5′ end of a J_{L} segment; and
ii) a CBE element within the nucleic acid sequence separating the 3′ end of a target V_{L} segment and the 5′ end of the first V_{L} segment which is 3′ of the target V_{L} segment.

2. The cell of claim 1, wherein the CBE element is located 5′ of at least one V segment in the locus.

3. The cell of claim 1, wherein the CBE element is in the same orientation as the target segment.

4. The cell of claim 1, wherein the CBE element is in the inverted orientation with respect to the target segment.

5. The cell of claim 1, wherein the CBE element is located 3′ of the V_{H} recombination signal sequence of the target V segment.

6. The cell of claim 1, wherein the target V_{H} or V_{L} segment is a non-native, exogenous, or engineered segment.

7. The cell of claim 6, wherein the cell is a mouse cell and the target V_{H} or V_{L} segment is a human segment.

8. The cell of claim 1, further comprising a non-native D_{H}, J_{H}, and/or I_{L} segment.

9. The cell of claim 8, wherein the non-native D_{H}, J_{H}, or I_{L} segment is a human segment.

10. The cell of claim 1, wherein the cell is a stem cell, embryonic stem cell, murine cell, murine stem cell, or murine embryonic stem cell.

11. The cell of claim 1, wherein the cell is heterozygous for the engineered IgH and/or IgL locus and the other IgH and/or IgL locus has been engineered to be inactive, wherein the cell will express an IgH and/or IgL chain only from the engineered IgH and/or IgL locus.

12. The cell of claim 1, further comprising
an engineered non-functional IGCR1 sequence in the IgH within the nucleic acid sequence separating the 3′ end of the 3′-most V_{H} segment of the IgH locus and the 5′ end of a D_{H} segment of the IgH locus.

13. The cell of claim 12, wherein the non-functional IGCR1 sequence comprises mutated CBE sequences; the CBE sequences of the IGCR1 sequence have been deleted; or the IGCR1 sequence has been deleted from the IgH locus.

14. The cell of claim 1, further comprising at least one of the following:
a) an IgL locus with human sequence;
b) a humanized IgL locus;
c) a human IgL locus;
d) an IgH locus with human sequence;
e) a humanized IgH locus;
f) a human IgH locus;
g) the engineered IgH locus further engineered to comprise only one V_{H} segment;
h) the engineered IgL locus further engineered to comprise only one V_{L} segment;
i) the IgL locus engineered to comprise one J_{L} segment;
j) an IgH locus engineered to comprise one J_{H} segment; and
k) an IgH locus engineered to comprise one DH segment.

15. The cell of claim 1, further comprising a mutation capable of activating, inactivating or modifying genes which lead to increased GC antibody maturation responses.

16. The cell of claim 1, further comprising a cassette targeting sequence in the target segment, which permits the replacement of the target segment.

17. The cell of claim 16, wherein the cassette targeting sequence is selected from the group consisting of:
an I-SceI meganuclease site; a Cas9/CRISPR target sequence; a Talen target sequence or a recombinase-mediated cassette exchange system.

18. The cell of claim 1, wherein the cell further comprises an exogenous nucleic acid sequence encoding TdT.

19. The cell of claim 18, further comprising a promoter operably linked to the sequence encoding TdT.

20. A genetically engineered mammal comprising the cell of claim 1.

21. A method of making an antibody, the method comprising the steps of:
injecting a mouse blastocyst with a cell of claim 1, wherein the cell is a mouse embryonic stem cell;
implanting the mouse blastocyst into a female mouse under conditions suitable to allow maturation of the blastocyst into a genetically engineered mouse;
isolating
1) an antibody; or
2) a cell producing an antibody;
from the genetically engineered mouse.
